(19) United States
(12) Patent Application Publication
(10) Pub. No.: US 2022/0154158 A1
(43) Pub. Date: May 19, 2022

(54) CAS9 VARIANTS WITH ENHANCED SPECIFICITY

(71) Applicant: Max-PLANCK-GESELLSCHAFT ZUR FÖRDERUNG DER WISSENSCHAFTEN E.V., München (DE)

(72) Inventors: Emmanuelle CHARPENTIER, Berlin (DE); Ines FONFARA, Berlin (DE); Majda BRATOVIC, Berlin (DE)

(21) Appl. No.: 17/437,504

(22) PCT Filed: Mar. 12, 2020
(86) PCT No.: PCT/EP2020/056639

§ 371 Date: Sep. 9, 2021

(30) Foreign Application Priority Data
Mar. 12, 2019 (EP) 19162150.7
Aug. 14, 2019 (EP) 19191840.8
Feb. 14, 2020 (EP) 20157371.4

(51) Int. Cl. C12N 9/22; C12N 15/11; C12N 15/90

(52) U.S. Cl. CPC C12N 9/22; C12N 15/11; C12N 15/907; A61K 38/00; C12N 2310/20; C12N 2320/30; C12N 2800/80

(57) ABSTRACT

The present invention relates to engineered Clustered Regularly Interspaced Short Palindromic Repeats (CRISPR)/CRISPR-associated protein 9 (Cas9) variants with enhanced specificity compared to wild type Cas9. The present invention also relates to compositions comprising one or more of those Cas9 variant(s), wherein the composition can be used for genome engineering. Furthermore, the present invention relates to pharmaceutical compositions comprising one or more of those Cas9 variant(s), wherein the pharmaceutical compositions can be used for treating disease(s), such as genetic disorders.

### FIELD OF THE INVENTION

The present invention relates to engineered Clustered Regularly Interspaced Short Palindromic Repeats (CRISPR)/CRISPR-associated protein 9 (Cas9) variants with enhanced specificity. The present invention also relates to compositions comprising one or more of those Cas9 variant(s), wherein the compositions can be used for genome engineering. Furthermore, the present invention relates to pharmaceutical compositions comprising one or more of those Cas9 variant(s), wherein the pharmaceutical compositions can be used for treating disease(s), such as genetic disorders.

### BACKGROUND OF THE INVENTION

The CRISPR (Clustered Regularly Interspaced Short Palindromic Repeats)-Cas (CRISPR-associated proteins) system is an adaptive immune system present in bacteria and archaea that protects against foreign genetic elements such as viruses and plasmids. CRISPR-Cas systems are classified into two major classes and six different types that are further divided into many subtypes (Makarova 2015 13 Nat. Rev. Microbiol. 722; Shmakov 2015 60 Mol. Cell 385; Shmakov 2017 Nat. Rev. Microbiol., 15(3):169-182.). The class 2 type II CRISPR-Cas system encompasses its effector protein Cas9.

The hallmark of CRISPR-Cas loci is the CRISPR array, which contains identical repeat sequences interspaced with spacer sequences that are derived from foreign nucleic acids and represent memory to previous infections. Adjacent to the CRISPR array is the cas operon, encoding Cas proteins necessary for immunity. Type II CRISPR systems contain an additional small non-coding RNA, named trans-activating CRISPR RNA (tracrRNA) (Deltcheva 2011 471 Nature 602.). CRISPR immunity is achieved through three phases, namely adaptation, CRISPR RNA (crRNA) biogenesis and interference (Hille 2016 371 Philos. Trans. R. Soc. B Biol. Sci. 20150496; Mohanraju 2016 353 Science aad5147; Wright 2016 164 Cell 29.). During the adaptation stage, a part of the foreign DNA is recognized and captured by Cas proteins, which then integrate it into the CRISPR array as a new spacer sequence (Jackson 2017 356 Science eaa15056.). This spacer sequence represents memory of the specific invader, and its storage in bacterial genome provides protection against subsequent infection with the same pathogen. The CRISPR array is expressed as a long precursor crRNA (pre-crRNA) consisting of many repeat-spacer units. The anti-repeat sequence of tracrRNA base pairs to each repeat of the pre-crRNA forming an RNA duplex that is bound by Cas9. The duplex is subsequently processed by the host endoribonuclease RNase III. This results in an intermediate tracrRNA:crRNA duplex, that is further processed to yield the mature tracrRNA:crRNA duplex bound to the effector protein Cas9. The two RNA molecules can be artificially fused into a so-called single-guide RNA (sgRNA; often also called “guide RNA”), containing the crRNA spacer (guide) and part of the repeat of tracrRNA (Jinek 2012 337 Science 816.). In order to identify the foreign DNA, Cas9 bound to guide RNA (tracrRNA:crRNA duplex or sgRNA) searches for a short sequence called protospacer adjacent motif (PAM). The PAM sequence is not present in the CRISPR array, which prevents Cas9 to target the bacterial chromosome, and thus enables the distinction between self and foreign DNA (Mojica 2009 155 Microbiology 733; Shah S A, 2013 10 RNA Biol. 891.). After PAM binding by Cas9 and target DNA strand separation, the crRNA spacer probes for complementarity with the target DNA (protospacer) (Anders C2014 513 Nature 569; Sternberg 2014 507 Nature 62.). Sufficient base pairing between the crRNA and target DNA leads to the formation of a stable R-loop, which is a structure where target strand of the DNA and crRNA are base-paired, while the non-target strand is displaced. This induces subsequent cleavage of the target and non-target strand by the HNH and RuvC endonuclease domains of Cas9, respectively, which results in a double-strand break (Jinek 2012 337 Science 816).

Efficient cleavage of the target DNA by Cas9 requires full complementarity between the crRNA and DNA in the so-called seed sequence. The seed sequence for Streptococcus pyogenes Cas9 comprises first 10-12 PAM-adjacent nucleotides (Jinek 2012 337 Science 816.). The seed sequence is one of the major determinants of Cas9 specificity. The more sensitive a Cas9 protein is to mismatches between the crRNA and DNA (i.e. the longer the seed sequence), the less off-target cleavage is expected to occur. Thus, natural or engineered Cas9 variants with longer seed sequence requirements should be more specific.

The simplicity and programmability of the CRISPR-Cas9 system has been widely adopted for numerous genome editing and engineering applications (Barrangou 2016 34 Nat. Biotechnol. 933; Dominguez 2016 17 Nat. Rev. Mol. Cell Biol. 5; Donohoue Trends Biotechnol., http://www.sciencedirect.com/science/article/pii/S0167779917301877 viewed 7 Aug. 2017; Doudna 2014 346 Science 1258096; Komor 2017 168 Cell 20; Singh 2017 599 Gene 1.). However, off-target cleavage by Cas9 makes the characterization of biochemical requirements for Cas9 specificity of particular interest. Several efforts to engineer Cas9 proteins with improved specificity have been made (Kleinstiver 2016 529 Nature 490; Slaymaker 2016 351 Science 84; Tycko 2016 63 Mol. Cell 355.).

Thus, the technical problem underlying the present invention is the provision of one or more of Cas9 proteins having improved specificity compared to wild type (wt) Cas9.

The technical problem is solved by provision of the embodiments as provided herein and as characterized in the claims.

### SUMMARY OF THE INVENTION

The present invention relates to the embodiments disclosed in the following items:
1. A Streptococcus pyogenes Cas9 (SpCas9) protein comprising or consisting of
   (i) a polypeptide with an amino acid sequence according to SEQ ID NO: 1 wherein the arginine at position 63 and the glutamine at position 768 are each replaced by alanine, or
   (ii) a polypeptide with an amino acid sequence having at least 90% sequence identity to the amino acid sequence according to SEQ ID NO: 1, wherein the residue corresponding to the arginine at position 63 of SEQ ID NO: 1 and the residue corresponding to the glutamine at position 768 of SEQ ID NO: 1 are each replaced by alanine, and wherein said polypeptide has enhanced specificity compared to a polypeptide with the amino acid sequence according to SEQ ID NO: 1.
2. The SpCas9 protein according to item 1(i) having enhanced specificity compared to a polypeptide with the amino acid sequence according to SEQ ID NO: 1.
3. The SpCas9 protein according to item 1 or 2 further comprising one or more mutations that decrease nuclease activity which are selected from:
   (i) D10A or D10N, and/or
   (ii) H840A, H840N or N840Y.
4. The SpCas9 protein according to any one of items 1 to 3 further comprising one or more nuclear localization signal(s) and/or one or more tag(s).
5. A polynucleotide encoding the SpCas9 protein according to any one of items 1 to 4.
6. The polynucleotide according to item 5, wherein said polynucleotide is codon-optimized for expression in a eukaryotic cell.
7. A vector comprising the polynucleotide according to item 5 or 6.
8. The vector according to item 7, wherein said polynucleotide is operably linked to one or more transcription regulatory element(s).
9. A host cell comprising the SpCas9 protein according to any one of items 1 to 4, and/or the polynucleotide according to item 5 or 6, and/or the vector of item 7 or 8.
10. A composition comprising a CRISPR complex, wherein the CRISPR complex comprises:
   (i) a guide RNA and the SpCas9 protein according to any one of items 1 to 4;
   (ii) a guide RNA and the polynucleotide according to item 5 or 6; or
   (iii) a guide RNA and the vector according to item 7 or 8.
11. The composition according to item 10, wherein the guide RNA is a single guide RNA or a tracrRNA:crRNA duplex.
12. The composition according to item 10 or 11 for use in treating a disease which is based on one or more mutation(s).
13. Method of treating a disease which is based on one or more mutation(s) comprising administering an effective amount of the composition according to item 10 or 11 to a subject in need of such a treatment.
14. The composition for the use according to item 12 or the method according to item 13, wherein the disease is based on one mutation in the genome.
15. The composition for the use according to item 12 or 14 or the method according to item 13 or 14, wherein the disease is an inheritable disease.
16. The composition for the use according to any one of items 12, 14 and 15, or the method according to any one of items 13 to 15, wherein the disease is achondroplasia, alpha-1 antitrypsin deficiency, Alzheimer's disease, antiphospholipid syndrome, autism, autosomal dominant polycystic kidney disease, cancer (such as breast cancer, colon cancer, prostate cancer, or skin cancer), Charcot-Marie-Tooth, cri du chat, Crohn's disease, cystic fibrosis, dercum disease, down syndrome, duane syndrome, duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan Syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, Wilms-Tumour-Aniridia-Syndrom (WAGR) or Wilson disease.
17. Use of the composition according to item 10 or 11 for genome engineering, provided that said use is not a method for treatment of the human or animal body by surgery or therapy, and provided that said use is not a method for modifying the germline genetic identity of human beings.
18. A method for genome engineering in a cell, wherein the method comprises any one of the following steps:
   (i) contacting said cell with a guide RNA and the SpCas9 protein according to any one of items 1 to 4; or
   (ii) expressing in said cell a guide RNA and the SpCas9 protein according to any one of items 1 to 4.
19. A pharmaceutical composition comprising
   (i) a guide RNA and the SpCas9 protein according to any one of items 1 to 4;
   (ii) a guide RNA and the polynucleotide according to item 5 or 6; or
   (iii) a guide RNA and the vector according to item 7 or 8.
   20. The pharmaceutical composition according to item 19 for use in treating a disease which is based on one or more mutation(s).
21. The pharmaceutical composition for the use according to item 20, wherein the disease is based on one mutation in the genome.
22. The pharmaceutical composition for the use according to item 20 or 21, wherein the disease is an inheritable disease.
23. The pharmaceutical composition according to any one of items 19 to 22 for use in treating achondroplasia, alpha-1 antitrypsin deficiency, Alzheimer's disease, antiphospholipid syndrome, autism, autosomal dominant polycystic kidney disease, cancer (such as breast cancer, colon cancer, prostate cancer, or skin cancer), Charcot-Marie-Tooth, cri du chat, Crohn's disease, cystic fibrosis, dercum disease, down syndrome, duane syndrome, duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan Syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, Wilms-Tumour-Aniridia-Syndrom (WAGR) or Wilson disease.

The invention is not restricted to the embodiments disclosed in the above items. The skilled person knows suitable alternatives which may be used and may thereby consult, e.g., the description and Examples provided below.

### DESCRIPTION OF THE DRAWINGS

FIG. 1. The influence of mismatches between the crRNA and DNA on cleavage and binding of S. pyogenes Cas9. A Scheme of speM target site (highlighted by a black square) used in the in vitro assays. Base pairing of the crRNA to the target strand is indicated (for reasons of clarity, crRNA is shown truncated and tracrRNA is not shown). The mismatches were introduced in the non-target strand and numbering of mismatches is based on the numbers indicated above the non-target strand. For the in vivo assay, mismatches were introduced in the spacer part of the sgRNA and numbering is according to the numbering indicated on the crRNA. The PAM is shown in bold. B Binding constants (KD) derived from EMSAs with increasing concentrations of Cas9_wt and two molar excess of dual-RNA on 90 bp 5′-32P labeled PCR products of the target sites used for kinetic cleavage assays. Data obtained from at least three independent experiments were fitted by non-linear regression analysis using Origin Software. Error bars are given as standard deviations (SD). C In vitro cleavage rates (k_{obs}) obtained by kinetic cleavage assays with 5 nM plasmid DNA (containing wild-type or mutated target site) and 10 nM Cas9 in complex with 20 nM wt dual-RNA. Mutations along the target site are indicated on the x-axis, starting from the first PAM-adjacent nucleotide. The cleavage rate k1_{obs} (black) represents disappearance of the supercoiled plasmid DNA, whereas the cleavage rate k2_{obs} (white) represents appearance of the linear DNA. In vitro cleavage rates (k_{obs}) obtained by kinetic cleavage assays with 5 nM plasmid DNA (containing wild-type or mutated target site) and 10 nM Cas9 in complex with 20 nM wt dual-RNA. Mutations along the target site are indicated on the x-axis, starting from the first PAM-adjacent nucleotide. The cleavage rate k1_{obs} (black) represents disappearance of the supercoiled plasmid DNA, whereas the cleavage rate k2_{obs} (white) represents appearance of the linear DNA. Data obtained from at least three independent experiments were fitted by non-linear regression analysis using Origin Software. Error bars are given as SD.

FIG. 2. Q768 is involved in Cas9 sensitivity to PAM-distal mismatches. A Bacterial survival assay with Cas9_wt, Cas9_Q768N, Cas9_Q768E and Cas9_Q768A, in the presence of wt or C15G sgRNAs. Survival represents the mean OD_{600 nm} of induced versus suppressed expression of wt or mutant Cas9. Higher values indicate better bacterial survival and less cleavage by Cas9, whereas lower values indicate poor survival and therefore more cleavage. B In vitro cleavage rates of Cas9_wt and Q768 mutants on target DNA having a mismatch at position 15 normalized to the cleavage rate of each protein on the wt substrate. Cleavage rates (k1_{obs} in black and k2_{obs} in white) were calculated as described in Methods. Error bars represent normalized standard deviation (SD) of at least three independent experiments. C-D Bacterial survival assay with Cas9_Q768A (C) and Cas9_Q768E (D), in the presence of wt and mismatched sgRNAs (labeled on y-axis). Data obtained from at least three independent experiments were fitted by non-linear regression analysis using Origin Software. Error bars are given as SD. Bars colored in black are values obtained with mismatched sgRNAs that are not more than 1.5 times the value obtained with wt sgRNA and designate that the protein is not sensitive to the mismatch. Bars colored in white stand for values obtained with mismatched sgRNAs that are more than 1.5 times higher than with wt sgRNA and suggest that the protein is sensitive to the mismatch. The dotted line at 0.95 indicates the value obtained with dCas9 (catalytically inactive Cas9) that represents no cleavage. Error bars are given as SD.

FIG. 3. Arginine residues from the bridge helix affect cleavage and binding by Cas9. A In vitro cleavage rates of Cas9_wt, Cas9_R63A and Cas9_R66A obtained by kinetic cleavage assays using 5 nM plasmid DNA (containing the wt target site) and 10 nM Cas9 in complex with 20 nM dual-RNA. k1_{obs} is shown in black and k2_{obs} in white. B Binding constants resulting from EMSAs of Cas9_wt, Cas9_R63A and Cas9_R66A on 90 bp 5′-³²P labeled PCR product of the wt target site. Data obtained from at least three independent experiments were fitted by non-linear regression analysis using Origin Software. Error bars are given as SD.

FIG. 4. Two groups of arginine residues with opposite effects on Cas9 sensitivity to mismatches. A Heat map of in vitro cleavage rates of Cas9_wt and Cas9_R63A normalized to each protein's rate on the wt substrate. Cleavage rates were determined as described in Methods from at least three independent experiments. Values less than 1 indicate that the cleavage rate is worse or similar to the cleavage rate on the wt substrate, whereas values more than 1 indicate that the cleavage rate is similar to or better than the cleavage rate on the wt substrate. B-C Binding constants of Cas9_R63A (B) or Cas9_R66A (C) on the wt and mismatched substrates, determined as described in FIG. 1B and calculated with Origin Software. Asterisks indicate that due to poor binding of the protein to the substrate, binding constants could not be determined with confidence under the tested conditions. Error bars represent the SD of at least three independent experiments.

FIG. 5. Arginine 63 stabilize the R-loop in the presence of mismatches. A Binding constants resulting from EMSAs of Cas9_wt, Cas9_R63A and Cas9_R66A on 140 bp 5′-³²P labeled oligonucleotides containing the NGG PAM (bold) and the target site (highlighted by a black square) either without a bubble (target and non-target strand fully base paired), a 5 nt bubble starting from the PAM-adjacent side of the target, or with a complete 20 nt bubble. The bubble was designed by introducing 5 or 20 mismatches between the target and non-target strand of the double-stranded oligonucleotide substrate. This resulted in partially or fully opened DNA substrate to which the crRNA can still fully base pair. B Binding constants of Cas9_wt (black) and Cas9_R63A (white) on the different oligonucleotide substrates (with the target site indicated on the x-axis). Positions where the substrate is opened is shown as a bubble and a mismatch is marked by an asterisk. Binding constants were determined as described previously. Error bars represent the SD of at least three independent experiments.

FIG. 6. Combination of R63A or R66A with Q768A in Cas9 enhances sensitivity to mismatches. Specificity of Cas9_wt (A), Cas9_R63A/Q768A (B) and Cas9_R66A/Q768A (C) Cas9_R63A (D), Cas9_R66A (E) and Cas9_Q768A (F) calculated as the ratio of survival in the presence of mismatched sgRNA versus survival in the presence of wt sgRNA (off-target/on-target). (G) shows the mean value for the specificities of Cas9_wt, Cas9_R63A, Cas9_R66A, Cas9_Q768A, Cas9_R63A/Q768A and Cas9_R66A/Q768A, normalized to Cas9_wt. Error bars represent normalized SD.

FIG. 7: Cas9 double mutant activity in eucaryotic cell lines. Cas9 genome editing was analyzed by targeting the epithelial cell adhesion molecule (EpCAM) with four different sgRNAs with either of Cas9_wt, Cas9_R63A/Q768A and Cas9_R66A/Q768A in (A) MCF7 or (B) HaCaT cell lines. The fraction of EpCAM-negative versus positive cells was detected and quantified 10 days post transfection of the plasmids that express the indicated sgRNA and the respective version of Cas9 using quantified using FITC-labelled EpCAM antibody. (C)-(D) shows the same results as relative editing between Cas9_wt and Cas9-mutant Cas9_R63A/Q768A (C) or SpCas9-wt and Cas9-mutant Cas9_R66A/Q768A (D).

FIG. 8. Representative plots showing the gating strategy in flow cytometry analysis. Dead cells and doublets were excluded from the analysis based on FSC-A/SSC-A scatter plot. Next, dead cells (PerCP-Cy5.5 positive) were further excluded based on staining with 7-AAD viability staining solution. Live cells (PerCP-Cy5.5 negative) were further gated on FITC to discriminate FITC negative (EpCAM negative, edited cells) from FITC positive (EpCAM positive, non-edited cells). Numbers indicate percentages of cells in each gate.

FIG. 9. Cas9_R63A/Q768A is an enhanced specificity Cas9 variant. a Percentage of EpCAM on-target editing in MCF-7 cells by Cas9_wt (black) and Cas9_R63A/Q768A (grey) in the presence of four different sgRNAs, determined by flow cytometry as described in Methods. b-c Percentage of EpCAM editing by Cas9_wt (black) and Cas9_R63A/Q768A (grey) in the presence of EpCAM-4 (c) and EpCAM-1 (d) sgRNAs, that were either fully complementary to the target site, or contained single mismatches to the PAM-distal part of the target site. Editing percentage was determined by flow cytometry as described in Methods.

FIG. 10. Target sites within EpCAM gene used for gene editing experiments. Scheme of EpCAM-4 (a, grey) and EpCAM-1 (b, black) target sites for gene editing experiments in MCF-7 cells. Base pairing of the sgRNA to the target strand is indicated (for reasons of clarity, sgRNA is shown truncated). The mutations were introduced in the spacer sequence of the sgRNA. The numbering of mismatches is based on the numbers indicated above the sgRNA. The PAM is circled.

FIG. 11: List of target sites subjected to amplicon sequencing.

Shown are the PAM (in bold) and the sequence for the on- and off-target sites for the different sgRNAs used for amplicon sequencing. Nucleotides in black correspond to the sgRNA, whereas Nucleotides in light grey are mismatches to the sgRNA.

FIG. 12. Amplicon Seq of VEGFA in Cas9_R63A/Q768A edited cells.

Percentage of on- and off-target editing in HEK293 cells by Cas9_wt (black) and Cas9_R63A/Q768A (grey) with sgRNAs targeting VEGFA3 (a) and VEGFA1 (b) sites, determined by amplicon sequencing, as described in Methods. OT stands for off-target. Error bars represent standard deviation of at least three independent experiments. Values from independent replicates are shown as black dots. Statistical significance between Cas9_R63A/Q768A and Cas9_wt was determined by a standard t-test (*p≤0.05, **p≤0.01, ***p≤0.001, ****p≤0.0001).

FIG. 13. Amplicon Seq of EMX1 in Cas9_R63A/Q768A edited cells.

Percentage of on- and off-target editing in HEK293 cells by Cas9_wt (black) and Cas9_R63A/Q768A (grey) with sgRNAs targeting EMX1.4 sites, determined by amplicon sequencing, as described in Methods. OT stands for off-target. Error bars represent standard deviation of at least three independent experiments. Values from independent replicates are shown as black dots. Statistical significance between Cas9_R63A/Q768A and Cas9_wt was determined by a standard t-test (*p≤0.05, **p≤0.01, ***p≤0.001, ****p≤0.0001).

### DETAILED DESCRIPTION OF THE INVENTION

The CRISPR-Cas System

The CRISPR-Cas system originates from bacteria and can be used for genome engineering (genome editing, targeted genome cleavage) in bacteria and eukaryotes (see, e.g., Jinek et al. 2012, Science 337, 816-821; Cong, Science 2013, 339:819-23; Mali, Science 2013, 339:823-26; Hwang, Nature Biotechnology 2013, 31:227-229; Jinek, Science 2013, 337:816-21; Doudna, Science 2014, 346 1258096; Hsu, Cell 2014, 157 1262-78; Sander Nat Biotechnol 2014, 32 347-55; Wang, Cell 2013, 153 910-8; Yang Cell 2013, 154 1370-9). Preferably, the Cas9 protein of the invention is used for genome engineering in eukaryotes, most preferably, the Cas9 protein of the invention is used for genome engineering in human. For example, as described in more detail below, Cas9 protein of the invention may be used for treating a disease, which is based on one or more mutation(s) in the genome. As explained below, such diseases comprise inheritable diseases which are based on one mutation(s) in the genome. As will be explained further below, in the CRISPR-Cas system, the Cas9 protein (i.e. the nuclease) forms a complex (CRISPR complex) with a guide RNA. The CRISPR-Cas system known in the art (and any utilization of the system) can likewise be used with the Cas9 protein of the present invention (i.e. wherein the commonly used Cas9 is replaced by the Cas9 of the present invention).

Before the CRISPR-Cas system was discovered, zinc finger nucleases (ZFNs) and/or transcription activator-like effector nucleases (TALENs) were used as site-specific DNA nucleases for genome engineering/editing (Li, Nature 2011, 475:217-221; Bedell, Nature 2012, 491:114-118; Genovese, Nature 2014, 510:235-240). However, the CRISPR-Cas system provides a much more simple system for genome engineering/editing method(s).

Methods of the CRISPR-Cas system (for biological applications, e.g. genome engineering) are described, e.g., in “CRISPR-Cas a laboratory manual” edited by Jennifer Doudna and Prashnat Mali (2016, by Cold Spring Harbor Laboratory Press, Cold Spring Harbor, N.Y.), which is incorporated herein in its entirety by reference. CRISPR-Cas can induce targeted DNA single- or double-strand breaks in the genome, which can then be repaired through either non-homologous end-joining (NHEJ) or homology-directed repair (HDR) pathways (Cox, Nat Med 2015, 21:121-31; Doudna, Science 2014, 346:1258096; Hsu, Cell 2014, 157:1262-78; Sander, Nat Biotechnol 2014, 32:347-55; Yang, Cell 2013, 154:1370-9). CRISPR-Cas mediated gene knock-out and knock-in rely on NHEJ and HDR. NHEJ-mediated gene knock-out is based on error-prone DNA repair of Cas9-mediated DNA double strand break and can be used to explore the effects of disrupting a particular gene. HDR-mediated gene knock-in enables precise genome editing including sequence insertion, deletion and replacement, which can be applied for many purposes such as visualization of endogenous gene products, modeling or correction of disease-related mutations etc.

Using Cas9 as nuclease has the advantage that it solely requires the expression of the Cas9 nuclease protein in combination with a guide RNA. The guide RNA as used herein can be a “single guide RNA”. The single guide RNA has a guide sequence (which can bind to a desired target sequence, e.g., in a genome) a tracr mate sequence and a tracrRNA, wherein said three components are in a single polynucleotide. The tracrRNA binds to the tracr mate sequence over a stretch of complementary nucleotides. The guide RNA sequence-specifically guides the Cas9 protein to the desired target sequence, e.g. in the genome, which is then cleaved by the Cas9 protein (nuclease). Thereby targeted cleavage of a desired sequence (e.g. in the genome of a desired cell/organism) is achieved. Thus, Cas9 is guided by a specificity-determining guide-RNA sequence (CRISPR RNA (crRNA)) that is associated with a trans-activating crRNA (tracrRNA) and forms Watson-Crick base pairs with the complementary DNA target sequence, resulting in site-specific double strand breaks (Heidenreich, 2016, Nature Reviews Neurosciences, 17: 36-44).

Besides the single guide RNA the Cas9 can be guided by a “tracrRNA:crRNA duplex”. Thereby, the crRNA encompasses a sequence corresponding to the guide sequence and a sequence corresponding to the tracr mate sequence. The tracrRNA is not covalently linked to the crRNA, but the tracrRNA binds to the tracr mate sequence so that the crRNA forms a duplex with the tracrRNA (i.e. the “tracrRNA:crRNA duplex”). Like the single guide RNA, the “tracrRNA:crRNA duplex” can sequence-specifically direct the Cas9 protein (thereby forming the CRISPR complex) to a desired target sequence (e.g. in a genome of a cell/organism) so that the target is cleaved (which can be exploited for genome engineering/editing).

Accordingly, a two-component system (consisting of Cas9 and a fusion of the tracrRNA-crRNA duplex to a “single guide RNA”, which may also be denominated “sgRNA”) or a simple three-component system (consisting of Cas9, a tracrRNA molecule and a crRNA molecule, wherein the two RNA molecules are forming a “tracrRNA:crRNA duplex”, which may also be denominated “dual-guided RNA”) can be engineered (forming the CRISPR complex) for expression in eukaryotic cells and can achieve DNA cleavage at any genomic locus of interest.

The term “target sequence specific CRISPR RNA (crRNA)”, as used herein, is commonly know in the art and described, e.g., in Makarova, Nat Rev Microbiol 2011, 9: 467-477; Makarova, Biol Direct 2011, 6:38; Bhaya, Annu Rev Genet 2011, 45:273-297; Barrangou, Annu Rev Food Sei Technol 2012, 3:143-162; Jinek, Science 2012, 337:816-821, Cong, Science 2013, 339:819-823; Mali, Science 2013, 339:823826 or Hwang, Nature Biotechnology 2013, 31:227-229. crRNAs differ depending on the Cas9 system but typically contain a sequence complementary to the target sequence(s) (or complementary to a part of the target sequence) of between 10 and 30, preferably between 15 and 25 (e.g. about 20) nucleotides length, flanked by two direct repeats (DR) of a length of between 21 to 46 nucleotides (tracr mate sequence(s)). The 3′ located DR of the crRNA is complementary to and hybridizes with the corresponding tracrRNA, which in turn binds to the Cas9 protein.

The term “trans-activating crRNA (tracrRNA)” is commonly known in the art and described, e.g., in Hsu, Cell 2014, 157:1262-78, Yang, Nature Protocols 2014, 9:1956-1968 and Heidenreich, Nature Reviews Neurosciences 2016, 17:36-44. The term “tracrRNA” refers to a small RNA, that is complementary to and base pairs with a crRNA, thereby forming an RNA duplex. The tracrRNA may also be complementary to and may base pair with a pre-crRNA, wherein this pre-crRNA is then cleaved by an RNA-specific ribonuclease, to form a crRNA:tracrRNA hybrid (duplex). In particular, the tracrRNA contains a sequence complementary to the palindromic repeat of the crRNA or of the pre-crRNA. Therefore it can hybridize to a crRNA or pre-crRNA with direct repeat. The tracrRNA is part of both the single guide RNA and the tracrRNA:crRNA duplex.

The skilled person readily knows how a tracrRNA:crRNA duplex (i.e. a guide RNA consisting of at least one target sequence specific CRISPR RNA (crRNA) molecule and at least one tracrRNA molecule) that target a desired target sequence (e.g. a desired protein encoding gene) can be designed. For example, such a dual-guide RNA may be designed by designing a crRNA and tracrRNA separately. A crRNA may be designed by a sequence that is complementary to the target sequence with a part or the entire DR sequence. A tracrRNA may be synthesized under the optimal promoter (e.g. U6 promoter) as shown by Jinek, Science, 337: 816-821.

The skilled person also knows, by consulting routine methods, how to design single guide RNAs (chimeric RNA molecules) comprising at least one target sequence specific crRNA and at least one tracrRNA (i.e. single guide RNAs or sgRNAs) that target a desired target sequence (e.g. a desired protein encoding gene). For example, such a single guide RNA may be designed by the fusion of a sequence that is complementary to the target sequence (or complementary to a part of the target sequence) of 10-30, preferably 15-25 (e.g. about 20) nucleotides in length with a part or the entire DR sequence and with a part or the entire of a tracrRNA, e.g. as shown by Jinek et al. 2012, Science 337, 816-821; Cong, Science 2013, 339:819-23; Mali, Science 2013, 339:823-26; Hwang, Nature Biotechnology 2013, 31:227-229; Jinek, Science 2013, 337:816-21. Within the single guide RNA a segment of the DR (=direct repeat, corresponding to the tracr mate sequence) and the tracrRNA sequence are complementary and are able to hybridize and to form a hairpin structure. A further method to obtain a single guide RNA is described, e.g., in Ran, Nat Protoc 2013, 8:2281-2308. As described below in more detail, in accordance with the present invention it is envisaged to complement the established computational tools (Labun, Nucleic Acids Res. 2016 Jul. 8; 44(W1):W272-6 (PMID 27185894); Haeussler; Genome Biol. 2016 Jul. 5; 17(1):148 (PMID 27380939)) that predict the “perfect” sgRNA with further experimental steps for validating the selected sgRNA.

The present invention makes use of the above-described CRISPR-Cas system. Thereby, the SpCas9 protein(s) of the present invention can form a CRISPR complex with a single guide RNA or a tracrRNA:crRNA duplex, so the genome engineering (targeted genome cleavage and desired genome engineering/editing/manipulation) can be accomplished. Thus, the present invention provides a composition comprising or consisting of a CRISPR complex comprising or consisting of a guide RNA and the SpCas9 protein as defined herein. The guide RNA can be a single guide RNA or a tracrRNA:crRNA duplex. The CRISPR complex can be used (in a method) for genome engineering. The use and/or methods for genome engineering can comprises contacting a cell with a guide RNA and the SpCas9 protein or expressing in a cell a guide RNA and the SpCas9 protein. The herein provided use and/or methods for genome engineering may be carried out in vitro. However, in the methods for genome engineering, the CRISPR complex can also be applied in vivo, to a subject, e.g. an animal or a human patient (for example in order to produce an animal model or for therapeutic applications).

Genome engineering with the CRISPR system (e.g. compositions comprising a CRISPR complex) is described in detail in the various publications referred to above, each of which is incorporated herein by reference with its entirety. The skilled person is aware of the genome engineering (editing/manipulation) methods in the art and is in the position to apply the Cas9 protein of the present invention to those methods. Thus, any of those methods in the art can likewise be used with the Cas9 protein of the present invention instead of the wild type (unaltered) Cas9. As used herein, genome engineering refers to, e.g. altering or manipulating the expression of one or more genes or the one or more gene products, in prokaryotic or eukaryotic cells, in vitro, in vivo or ex vivo. Preferably, genome engineering refers to altering or manipulating the expression of one or more (e.g. 2 or 3) genes in a eukaryotic cell. For example, the Cas9 protein of the invention can be used for altering the expression of a gene in human cells, as described herein above and below. Genome engineering can refer to a process of modifying a target nucleic acid. Genome engineering can refer to the integration of non-native nucleic acid into native nucleic acid. Genome engineering can refer to the site-directed modification of a target nucleic acid (e.g. a target gene) by using a Cas9 polypeptide and a guide RNA, without integration or deletion of the target nucleic acid (e.g. the target gene). Genome engineering can refer to the cleavage of a target nucleic acid, and the rejoining of the target nucleic acid without an integration of an exogenous sequence in the target nucleic acid, or without a deletion in the target nucleic acid. The native nucleic acid can comprise a gene. The non-native nucleic acid can comprise a donor template polynucleotide as defined below. In the methods described herein, the Cas9 of the present invention can introduce double-stranded breaks in nucleic acid, (e.g. genomic DNA). The double-stranded break can stimulate a cell's endogenous DNA-repair pathways (e.g. HDR and/or NHEJ, or A-NHEJ (alternative non-homologous end-joining)). Mutations, deletions, alterations, and integrations of foreign, exogenous, and/or alternative nucleic acid can be introduced into the site of the double-stranded DNA break.

Herein HDR refers to a mechanism in cells to repair single or double strand DNA lesions by homologous recombination (see, e.g., Cong, Science 2013, 339:819-23; Pardo, Cellular and Molecular Life Sciences 2009, 66:1039-1056; Bolderson, Clinical Cancer Research 2009, 15:6314-6320). The HDR repair mechanism can only be used by the cell when there is a homologue piece of DNA (i.e. a donor template polynucleotide) present in the nucleus. Alternatively, NHEJ can take place. The highly error-prone NHEJ pathway induces insertions and deletions (indels) of various lengths that can result in frameshift mutations and, consequently, gene knockout. By contrast, the HDR pathway directs a precise recombination event between a homologous DNA donor template (i.e. a donor template polynucleotide) and the damaged DNA site, resulting in accurate correction of the single or double strand break. Therefore, HDR can be used to introduce specific mutations or transgenes into the genome. The donor template polynucleotide (usually a ssODN) has to contain a region with sequence homology with the region to be repaired. The term “homologous recombination” refers to a mechanism of genetic recombination in which two DNA strands comprising similar nucleotide sequences exchange genetic material. Cells use homologous recombination for the repair of damaged DNA, in particular for the repair of single and double strand breaks. The mechanism of homologous recombination is well known to the skilled person and has been described, for example by Paques, Microbiol Mol Biol Rev 1999, 63:349404.

In the appended Examples gene editing experiments in the human breast cancer cell line MCF-7 were performed, wherein the oncogene EpCAM was targeted for deletion. In this regard, it was decided to select EpCAM due to its function as an oncogene and its potential as relevant clinical target. The appended Examples confirm that this oncogene can be targeted for deletion with the Cas9 protein of the invention. The appended Examples also show editing of the oncogene VEGFA. Increased expression of VEGFA is correlated with tumor development, and thus, VEGFA is considered as a relevant target for novel cancer treatment strategies. Accordingly, the appended Examples demonstrate that the Cas9 protein of the invention can be used for (partially or completely) deleting or inactivating one or more oncogene(s) from the genome of human cells. Therefore, the Cas9 protein of the invention may be used for deleting or inactivating one or more oncogene(s) from the genome of human cells, e.g. for preventing or treating cancer. The term “oncogene” is commonly known in the art and relates to a gene which promotes cancer development and/or cancer growth when it is overexpressed. The meaning of the term “overexpression” is also commonly known in the art and refers to the abnormal expression of a gene in increased quantity. Thus, the term “overexpression” includes the abnormal increased expression of a given gene as compared to the expression of the same gene in corresponding healthy reference tissue. In line with this, the Cas9 protein of the invention may be used for introducing one or more tumor suppressor gene(s) into the genome of human cells, e.g. for preventing or treating cancer. The term “tumor suppressor gene” is commonly known in the art and relates to a gene the product of which inhibits cancer development and/or cancer growth.

In the herein provided method(s) multiple guide RNAs (single guide RNAs and/or tracrRNA:crRNA duplexes) can be used (in concert with the Cas9 of the present invention) to target several genes at once (multiplexing). This method may allow editing of multiple genes (simultaneously), e.g., for studying genetic interactions, or treating or modeling multigenic disorders. For example, 2 to 10, preferably 2 to 3, most preferably 2 different guide RNAs or 2 to 10, preferably 2 to 3, most preferably 2 different polynucleotides encoding different guide RNAs (i.e. single- or dual-guide RNAs) may be used in context of the present invention. Also, it is envisaged that one or more single guide RNAs and/or one or more tracrRNA:crRNA duplexes are used together in a CRISPR complex described herein (i.e. with the SpCas9 of the invention). For instance, one single guide RNA and one tracrRNA:crRNA duplex are used together for multiplexing. Also, two single guide RNAs and two tracrRNA:crRNA duplexes may be used together for multiplexing. Also, one single guide RNA and two tracrRNA:crRNA duplexes are used together for multiplexing. Also, two single guide RNAs and one tracrRNA:crRNA duplex may be used together for multiplexing.

Successful genome engineering with the Cas9 protein of the present invention are well known in the art and include, without limitation, assays based on physical separation of nucleic acid molecules, sequencing assays as well as cleavage and digestion assays and DNA analysis by the polymerase chain reaction (PCR). Examples for assays based on physical separation of nucleic acid molecules include MALDI-TOF, denaturating gradient gel electrophoresis and other such methods known in the art, see for example Petersen, Hum Mutat 2002, 20:253-259; Hsia, Theor, Appl Genet 2005 111:218-225; Tost, Clin Biochem 2005, 35:335-350; Palais, Anal Biochem 2005, 346:167-175. Examples for sequencing assays comprise, without limitation, approaches of sequence analysis by direct sequencing, fluorescent SSCP in an automated DNA sequencer and Pyrosequencing. These procedures are common in the art, see e.g. Adams (Ed.), “Automated DNA Sequencing and Analysis”, Academic Press, 1994; Alphey, “DNA Sequencing; From Experimental Methods to Bioinformatics”, Springer Verlag Publishing, 1997; Ramon, J Transl Med 2003, 1:9; Meng, J Clin Endocrinol Metab 2005, 90:3419-3422. Examples for cleavage and digestion assays include without limitation restriction digestion assays such as restriction fragments length polymorphism assays (RFLP assays), Rnase protection assays, assays based on chemical cleavage methods and enzyme mismatch cleavage assays, see e.g. Youil, Proc Natl Acad Sci USA 1995, 92:87-91; Todd, J Oral Maxil Surg 2001, 59:660-667; Amar, J Clin Microbiol 2002, 40:446-452.

Besides the above, the skilled person knows various further applications and modifications of the CRSIPR-Cas system which can be used with the Cas9 protein of the present invention.

The Cas9 Protein of the Present Invention

The Cas9 protein (also called “Cas9 nuclease” or “Cas9 endonuclease”) refers to the “clustered regularly interspaced short palindromic repeats (CRISPR)-associated protein 9”.

Cas9 is well known in the art and has been described, e.g., in Heidenreich, Nature Reviews Neurosciences 2016, 17:36-44; Makarova, Nat Rev Microbiol 2011, 9:467-477 and in Makarova, Biol Direct 2011, 6:38. Cas9 proteins constitute a family of enzymes that require a base-paired structure formed between an activating tracrRNA and a targeting crRNA to cleave target single or double strand DNA. Cas9 can sequence specifically be directed with a single (chimeric) guide RNA or a tracrRNA:crRNA duplex to a desired target sequence to be cleaved, as described above. Most Cas9 nucleases introduce double strand breaks, but some previous studies used mutant Cas9 to introduce multiple single strand breaks to perform HDR-mediated genome editing in vitro. Site-specific cleavage by Cas9 occurs at locations determined by both base-pairing complementarity between the crRNA and the target DNA (the guide sequence binding to a desired target sequence) and a short motif, referred to as the protospacer adjacent motif (PAM), juxtaposed to the complementary region in the target DNA (see, e.g., Jinek, Science 2012, 337:816-821). The PAM target sequences of various CRISPR nucleases and their variants (e.g. 5′-NGG for SpCas9, 5′-NNGRRT for SaCas9, 5′-TTN for Cpf1) abundantly exist in the mammalian genome. Therefore, most genes can be targeted by using the herein provided means and methods without introducing a PAM sequence. However, in the event that there is no PAM sequence immediately downstream of the desired cleavage site, a PAM sequence (e.g. 5′-NGG for SpCas9, 5′-NNGRRT for SaCas9, 5′-TTN for Cpf1) may be introduced downstream of the desired cleavage site. Thus, depending on the used site-specific nuclease (e.g. Cas9) or nickase (e.g. Cas9 nickase), if not present at the target sequence (e.g. within the gene of interest) at the desired position/location in, e.g., the genome of a cell, a recognition site (a PAM sequence) for cleavage may be engineered at the target sequence/into the gene of interest.

Preferably, the Cas9 protein of the present invention is derived from the Streptococcus pyogenes Cas9 protein (SpCas9). Accordingly, the wild type Cas9 protein is preferably the Streptococcus pyogenes Cas9 (SpCas9) protein. The (wild type (wt)) SpCas9 protein has the sequence as shown in SEQ ID NO: 1. The Cas9 protein of the present invention has amino acid substitutions/replacements at specific sites in the amino acid sequence of the wild type Cas9 protein (i.e. in the Cas9 polypeptide). The terms “replaced” and “substituted” or “substitution” and “replacement” are used interchangeably herein. Thus, replacing an amino acid with another amino acid means that the amino acid is substituted by another amino acid.

Preferably, in the amino acid sequence of the Cas9 protein of the present invention, two amino acids are replaced/substituted. In particular, in the Cas9 protein of the present invention, two amino acids are replaced/substituted in the amino acid sequence of the wild type Cas9 protein. Thus, in the Cas9 (SpCas9) protein of the present invention, two amino acids in the amino acid sequence having SEQ ID NO: 1 are replaced/substituted by other amino acids.

Preferably, the Cas9 protein (SpCas9) of the present invention comprising or consisting of (i) a polypeptide with an amino acid sequence according to SEQ ID NO: 1 wherein the arginine at position 63 and the glutamine at position 768 are each replaced by alanine; or
(ii) a polypeptide with an amino acid sequence having at least 90% sequence identity to the amino acid sequence according to SEQ ID NO: 1, wherein the residue corresponding to the arginine at position 63 of SEQ ID NO: 1 and the residue corresponding to the glutamine at position 768 are each replaced by alanine, and wherein said polypeptide has enhanced specificity compared to a polypeptide with the amino acid sequence according to SEQ ID NO: 1.

Thus, in order to obtain the Cas9 protein of the present invention, the amino acid sequence of the wild type Cas9 protein (i.e. SEQ ID NO: 1) is altered at two distinct amino acid positions. Those positions are positions 63 and 768. The amino acid which is replaced/substituted is arginine at position 63 and glutamine at position 768. At each of said positions, arginine or glutamine is preferably replaced/substituted by alanine.

Accordingly, in the Cas9 protein of the present invention, the amino acids at positions 63 and 768 of the wild type Cas9 protein (i.e. SEQ ID NO: 1) are preferably each replaced/substituted by alanine.

The Cas9 protein of the present invention preferably comprises or consists of a polypeptide with an amino acid sequence according to SEQ ID NO: 1 wherein the arginine at position 63 and the glutamine at position 768 are each replaced by alanine (i.e. SEQ ID NO: 2).

The Cas9 protein(s) of the present invention has/have enhanced (“improved” or “increased” which terms can be used interchangeably with “enhanced”) specificity compared to the polypeptide with the amino acid sequence according to SEQ ID NO: 1 (i.e. the wild type Streptococcus pyogenes Cas9 protein). Accordingly, the Cas9 protein of the present invention has enhanced specificity compared to the wild type SpCas9 protein (which has the amino acid sequence according to SEQ ID NO: 1).

In accordance with the present invention, enhanced specificity means that the Cas9 protein of the present invention cleaves the target sequence with enhanced (higher/increased/improved) specificity compared to the protein/polypeptide having/with the amino acid sequence according to SEQ ID NO: 1/wild type Cas9/SpCas9 protein/polypeptide. More specifically, enhanced specificity means that the Cas9 protein of the present invention cleaves the target sequence with enhanced (higher/increased/improved) specificity to mismatches when compared to the polypeptide with the amino acid sequence of SEQ ID NO: 1 (i.e. wild type SpCas9) for most sgRNAs. Accordingly, the Cas9 protein of the present invention has enhanced nuclease specificity compared to the protein/polypeptide with the amino acid sequence according to SEQ ID NO: 1/wild type Cas9/SpCas9 protein/polypeptide. Enhanced specificity means that the Cas9 protein of the present invention produces less off-target mutations compared to the protein/polypeptide with the amino acid sequence according to SEQ ID NO: 1/wild type Cas9/SpCas9 protein/polypeptide. More specifically, enhanced specificity means that the Cas9 protein of the present invention produces less off-target mutations when compared to the polypeptide with the amino acid sequence of SEQ ID NO: 1 (i.e. wild type SpCas9) for most sgRNAs. Enhanced specificity means that the Cas9 protein of the present invention cleaves target sites which actually should not be cleaved less often compared the protein/polypeptide with the amino acid sequence according to SEQ ID NO: 1/wild type Cas9/SpCas9 protein/polypeptide. Enhanced specificity means that the CRISPR complex/CRISPR-Cas system with the Cas9 protein of the present invention cleaves less often at sites where the CRISPR complex/CRISPR-Cas system binds at imperfectly matched target sites (compared the CRISPR complex/CRISPR-Cas system with the protein/polypeptide with the amino acid sequence according to SEQ ID NO: 1/wild type Cas9/SpCas9 protein/polypeptide). Enhanced specificity means that the CRISPR complex/CRISPR-Cas system with the Cas9 protein of the present invention produces less off-target mutations at sites where the CRISPR complex/CRISPR-Cas system binds at imperfectly matched target sites (compared the CRISPR complex/CRISPR-Cas system with the protein/polypeptide with the amino acid sequence according to SEQ ID NO: 1/wild type Cas9/SpCas9 protein/polypeptide). Enhanced specificity means that the Cas9 protein of the present invention has decreased cleavage/nuclease activity as to off-target sites (compared to the protein/polypeptide with the amino acid sequence according to SEQ ID NO: 1/wild type Cas9/SpCas9 protein/polypeptide). An off-target site is a (target) site in the genome/DNA to which the guide RNA (singe guide RNA or tracrRNA:crRNA duplex) unspecifically binds and to which the Cas9 protein is unintentionally directed for cleavage.

The SpCas9 of the present invention has a specificity that is at least 1.5 times enhanced/higher compared to the specificity of the polypeptide with the amino acid sequence according to SEQ ID NO: 1 (i.e. wild type SpCas9).

The SpCas9 of the present invention has a specificity that is at least 2 times enhanced/higher compared to the specificity of the polypeptide with the amino acid sequence according to SEQ ID NO: 1.

In a preferred embodiment, the SpCas9 of the present invention has a specificity that is at least 2.2 times enhanced/higher compared to the specificity of the polypeptide with the amino acid sequence according to SEQ ID NO: 1.

In a preferred embodiment, the SpCas9 of the present invention has a specificity that is at least 2.5 times enhanced/higher compared to the specificity of the polypeptide with the amino acid sequence according to SEQ ID NO: 1.

In a more preferred embodiment, the SpCas9 of the present invention has a specificity that is at least 2.22 times enhanced/higher compared to the specificity of the polypeptide with the amino acid sequence according to SEQ ID NO: 1.

In an even more preferred embodiment, the SpCas9 of the present invention has a specificity that is at least 2.224 times enhanced/higher compared to the specificity of the polypeptide with the amino acid sequence according to SEQ ID NO: 1.

The SpCas9 of the present invention has a 150% enhanced/higher specificity compared to the polypeptide with the amino acid sequence according to SEQ ID NO: 1.

The SpCas9 of the present invention has a 200% enhanced/higher specificity compared to the polypeptide with the amino acid sequence according to SEQ ID NO: 1.

In a preferred embodiment, the SpCas9 of the present invention has a 220% enhanced/higher specificity compared to the polypeptide with the amino acid sequence according to SEQ ID NO: 1.

In a preferred embodiment, the SpCas9 of the present invention has a 250% enhanced/higher specificity compared to the polypeptide with the amino acid sequence according to SEQ ID NO: 1.

In a more preferred embodiment, the SpCas9 of the present invention has a 222% enhanced/higher specificity compared to the polypeptide with the amino acid sequence according to SEQ ID NO: 1.

In an even more preferred embodiment, the SpCas9 of the present invention has a 222.4% enhanced/higher specificity compared to the polypeptide with the amino acid sequence according to SEQ ID NO: 1.

The Cas9 protein of the present invention cleaves the target sequence with at least 1.5 times enhanced (higher/increased/improved) specificity when compared to the polypeptide with the amino acid sequence of SEQ ID NO: 1.

The Cas9 protein of the present invention cleaves the target sequence with at least 2 times enhanced (higher/increased/improved) specificity when compared to the polypeptide with the amino acid sequence of SEQ ID NO: 1.

In a preferred embodiment, the Cas9 protein of the present invention cleaves the target sequence with at least 2.2 times enhanced (higher/increased/improved) specificity when compared to the polypeptide with the amino acid sequence of SEQ ID NO: 1.

In a preferred embodiment, the Cas9 protein of the present invention cleaves the target sequence with at least 2.5 times enhanced (higher/increased/improved) specificity when compared to the polypeptide with the amino acid sequence of SEQ ID NO: 1.

In a more preferred embodiment, the Cas9 protein of the present invention cleaves the target sequence with at least 2.22 times enhanced (higher/increased/improved) specificity when compared to the polypeptide with the amino acid sequence of SEQ ID NO: 1.

In an even more preferred embodiment, the Cas9 protein of the present invention cleaves the target sequence with at least 2.224 times enhanced (higher/increased/improved) specificity when compared to the polypeptide with the amino acid sequence of SEQ ID NO: 1.

The Cas9 protein of the present invention cleaves the target sequence with mismatches at positions 10-20 with at least 1.5 times enhanced (higher/increased/improved) specificity to mismatches when compared to the polypeptide with the amino acid sequence of SEQ ID NO: 1.

The Cas9 protein of the present invention cleaves the target sequence with mismatches at positions 10-20 with at least 2 times enhanced (higher/increased/improved) specificity to mismatches when compared to the polypeptide with the amino acid sequence of SEQ ID NO: 1.

In a preferred embodiment, the Cas9 protein of the present invention cleaves the target sequence with mismatches at positions 10-20 with at least 2.2 times enhanced (higher/increased/improved) specificity to mismatches when compared to the polypeptide with the amino acid sequence of SEQ ID NO: 1.

In a preferred embodiment, the Cas9 protein of the present invention cleaves the target sequence with mismatches at positions 10-20 with at least 2.5 times enhanced (higher/increased/improved) specificity to mismatches when compared to the polypeptide with the amino acid sequence of SEQ ID NO: 1.

In an even preferred embodiment, the Cas9 protein of the present invention cleaves the target sequence with mismatches at positions 10-20 with at least 2.22 times enhanced (higher/increased/improved) specificity to mismatches when compared to the polypeptide with the amino acid sequence of SEQ ID NO: 1.

In the most preferred embodiment, the Cas9 protein of the present invention cleaves the target sequence with mismatches at positions 10-20 with at least 2.22 times enhanced (higher/increased/improved) specificity to mismatches when compared to the polypeptide with the amino acid sequence of SEQ ID NO: 1.

There can also be situations in which the above-mentioned specificity of the Cas9 protein of the present invention is at least 3 times higher compared to the polypeptide with the amino acid sequence of SEQ ID NO: 1.

There can also be situations in which the above-mentioned specificity of the Cas9 protein of the present invention is at least 4 times higher compared to the polypeptide with the amino acid sequence of SEQ ID NO: 1.

There can also be situations in which the above-mentioned specificity of the Cas9 protein of the present invention is at least 5 times higher compared to the polypeptide with the amino acid sequence of SEQ ID NO: 1.

There can also be situations in which the above-mentioned specificity of the Cas9 protein of the present invention is at least 6 times higher compared to the polypeptide with the amino acid sequence of SEQ ID NO: 1.

There can also be situations in which the above-mentioned specificity of the Cas9 protein of the present invention is at least 7 times higher compared to the polypeptide with the amino acid sequence of SEQ ID NO: 1.

There can also be situations in which the above-mentioned specificity of the Cas9 protein of the present invention is at least 8 times higher compared to the polypeptide with the amino acid sequence of SEQ ID NO: 1.

There can also be situations in which the above-mentioned specificity of the Cas9 protein of the present invention is at least 9 times higher compared to the polypeptide with the amino acid sequence of SEQ ID NO: 1.

There can also be situations in which the above-mentioned specificity of the Cas9 protein of the present invention is at least 10 times higher compared to the polypeptide with the amino acid sequence of SEQ ID NO: 1.

Under certain settings, the Cas9 protein of the present invention has specificity towards certain mismatched sgRNA that is up to 10 times enhanced/higher compared to the specificity of the polypeptide with the amino acid sequence according to SEQ ID NO: 1.

Under certain settings, the Cas9 protein of the present invention cleaves the target sequence with mismatches with up to 10 times enhanced (higher/increased/improved) specificity to mismatches when compared to the polypeptide with the amino acid sequence of SEQ ID NO: 1.

All of the above values can be supplemented with the term “about” in front of the indicated value.

Unspecific binding of the guide RNA can occur when, e.g., one (or 2 or 3 or 4 etc.) nucleotide(s) in the guide sequence do/does not match to the nucleotide sequence of the target sequence. For instance, when the guide sequence is 20 nucleotides in length, said guide sequence may unspecifically bind to a target sequence in the genome which is complementary to only 19 nucleotides of said guide sequence. In this case, the guide sequence has 95% identity with the target sequence in the genome. When such unspecific binding occurs, the Cas9 is directed to this undesired target site (which has only 95% identity) where the Cas9 should actually not cleave (i.e. the Cas9 can produce off-target effects at this undesired target site). With the Cas9 protein of the present invention, such unspecific binding and cleavage (off-target effects) are reduced which results in enhanced specificity. Indeed, with the Cas9 protein of the present invention, such unspecific binding and cleavage (off-target effects) are reduced for most sgRNAs, which results in enhanced specificity.

The enhanced specificity can be determined by the skilled person by using methods known in the art and by consulting, e.g., the Examples of the present invention. For example, for testing whether a given Cas9 protein has an enhanced specificity as compared to wild type SpCas9 (i.e. a polypeptide having the amino acid sequence of SEQ ID NO: 1), a kinetic cleavage assay as described below in the appended Examples may be performed.

In the appended Examples it could advantageously be shown that the Cas9 protein of the invention (e.g. SpCas9 comprising the mutations R63A and Q768A) displays increased specificity for different sgRNAs targeting different genes as compared to Cas9 wild type. However, it was also observed that for one specific sgRNA the Cas9 protein of the invention has a slightly decreased specificity when compared to Cas9 wild type. This can be explained as follows. It is well known since the beginning of Cas9 applications that the sequence of the sgRNA alone can affect specificity independent of Cas9 features (Wu, Quant Biol. 2014 June; 2(2):59-70 (PMID: 25722925)). Although this effect has been described for a long time, it is still poorly understood and several mechanisms have been proposed.

However, in accordance with the present invention it is envisaged that sgRNAs are selected, which (in all likelihood) do not lead to a decreased specificity of the Cas9 protein of the invention. More specifically, it is suggested herein to complement the established computational tools (Labun, Nucleic Acids Res. 2016 Jul. 8; 44(W1):W272-6 (PMID 27185894); Haeussler; Genome Biol. 2016 Jul. 5; 17(1):148 (PMID 27380939)) that predict the “perfect” sgRNA with further experimental steps for validating the selected sgRNA. For example, in accordance with the present invention, the selected sgRNA may be used for genome engineering in test cells, test tissue and/or test non-human animals, and said genome engineering step may be followed by whole-genome sequencing and/or double stranded break capture. Based on the obtained results an sgRNA may be selected which is not (or least) associated with off-target effects. These additional experimental steps advantageously promote the identification of ideal sgRNA that can be considered safe for therapeutic applications. In this approach it is feasible to test several Cas9 variants for their specificity, and to select the Cas9 variant which shows the highest specificity for the desired target and the selected sgRNA. However, in accordance with the present invention, it is envisaged to use the Cas9 protein of the invention (e.g. Cas9 comprising the mutations R63A and Q768A) for genome engineering, since the appended Examples demonstrate that Cas9_R63A/Q768A is more specific for the majority of sgRNAs as compared to Cas9 wild type. Therefore, the Cas9 protein of the invention should be used instead of Cas9 wild type for biomedical applications.

The skilled person knows the methods which can be used for effecting amino acid replacements/substitutions (in the wild type Cas9 protein/polypeptide in order to engineer/produce the Cas9 protein of the present invention). For instance, site-directed mutagenesis can be employed which is achieved with modified PCR techniques (PCR mutation) (QuickChange Kit, Stratagene; Kunkel, Proc Natl Acad Sci USA1985, 82(2):488-492; Vandeyar, Gene 65(1):129-133; Hashimoto-Gotoh, Gene 1995, 152: 271-275; Zoller, Methods Enzymol 1983, 100:468-500; Kramer, Nucleic Acids Res. 1984 12: 9441-9456) or the cassette mutation method, but are not limited to these methods. The methods are used to replace/substitute an individual amino acid with another amino acid. Other methods for amino acid substitution are known in the art and by the skilled person and can be employed for effecting desired amino acid replacements/substitutions so the Cas9 protein of the present invention is produced.

The Cas9 protein can also have additional amino acid substitutions/replacements, besides the specific amino acid substitutions/replacements defined above. For instance, the Cas9 protein of the present invention can comprises or consists of a polypeptide with an amino acid sequence having at least (about/approximately) 90% sequence identity to the amino acid sequence according to SEQ ID NO: 1 (wild type Cas9 from Streptococcus pyogenes), wherein the residue corresponding to the arginine at position 63 of SEQ ID NO: 1 and the residue corresponding to the glutamine at position 768 of SEQ ID NO: 1 are each replaced by alanine. Thus, besides the amino acids substitutions/replacements in SEQ ID NO: 1 at position 63 and the glutamine at position 768 with alanines, other additional amino acids can be replaced/substituted, so that the Cas9 protein of the present invention has at least (about/approximately) 90% sequence identity to the amino acid sequence according to SEQ ID NO: 1 (wild type Cas9 from Streptococcus pyogenes). Accordingly, the Cas9 protein of the present invention can also comprises or consists of a polypeptide with an amino acid sequence having at least (about/approximately) 90% sequence identity to the amino acid sequence according to SEQ ID NO: 1.

The Cas9 protein of the present invention can also have higher %-sequence identity (than (about/approximately) 90% as defined above) to the amino acid sequence according to SEQ ID NO: 1. Specifically, the Cas9 protein of the present invention as defined above can comprises or consists of a polypeptide with an amino acid sequence having at least (about/approximately) 91%, at least (about/approximately) 92%, at least (about/approximately) 93%, at least (about/approximately) 94%, at least (about/approximately) 95%, at least (about/approximately) 96%, at least (about/approximately) 97%, at least (about/approximately) 98% or at least (about/approximately) 99% sequence identity to the amino acid sequence according to SEQ ID NO: 1. In those Cas9 proteins, the amino acid substitutions/replacements at the residues corresponding to positions 63 and 768 of SEQ ID NO: 1) are present, as defined above. Preferably, the Cas9 protein of the present invention can comprises or consists of a polypeptide with an amino acid sequence having at least (about/approximately) 95% sequence identity to the amino acid sequence according to SEQ ID NO: 1. More preferably, the Cas9 protein of the present invention comprises or consists of a polypeptide with an amino acid sequence having at least (about/approximately) 96% sequence identity to the amino acid sequence according to SEQ ID NO: 1. More preferably, the Cas9 protein of the present invention comprises or consists of a polypeptide with an amino acid sequence having at least (about/approximately) 97% sequence identity to the amino acid sequence according to SEQ ID NO: 1. More preferably, the Cas9 protein of the present invention comprises or consists of a polypeptide with an amino acid sequence having at least (about/approximately) 98% sequence identity to the amino acid sequence according to SEQ ID NO: 1. Even more preferably, the Cas9 protein of the present invention comprises or consists of a polypeptide with an amino acid sequence having at least (about/approximately) 99% sequence identity to the amino acid sequence according to SEQ ID NO: 1. In accordance with the definition above, the above-mentioned Cas9 proteins of the present invention have enhanced specificity compared to a polypeptide with the amino acid sequence according to SEQ ID NO: 1. In any of these Cas9 proteins, the amino acid substitutions/replacements at positions 63 and 768 according to SEQ ID NO: 1 are present, as defined above (replacement/substitution of arginine or glutamine, respectively, at each of said positions with alanine).

To determine the percent identity of two sequences, the sequences are aligned for optimal comparison purposes (e.g., gaps can be introduced in one or both of a first and a second amino acid sequence for optimal alignment and non-homologous sequences can be disregarded for comparison purposes). The percent identity between the two sequences is a function of the number of identical positions shared by the sequences, taking into account the number of gaps, and the length of each gap, which need to be introduced for optimal alignment of the two sequences. Percent identity between two polypeptides/amino acid sequences is determined in various ways which are known by the skilled person, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482 489 (1981)) as incorporated into GeneMatcher Plus™, Schwarz and Dayhof (1979), Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. For purposes of the present invention, the comparison of sequences and determination of percent identity between two sequences can be accomplished using a Blossum 62 scoring matrix (with a gap penalty of 12, a gap extend penalty of 4, and a frameshift gap penalty of 5).

Besides the amino acid substitutions/replacements mentioned above, the arginine at position 63 and glutamine at position 768 described above in the wild type Cas9 protein (SpCas9) can also be replaced by other amino acids than alanines (in order to obtain Cas9 proteins having enhanced specificity).

The appended Examples surprisingly show that the Cas9_R63A/Q768A mutant has an increased specificity as compared to wild type Cas9. The appended Examples further show (beside the substitution of position Q768 with alanine) that also the substitution of Q768 with glutamate (E) or asparagine (N) increases specificity of the mutated Cas9 as compared to wild type Cas9. In this regard, the specificity could be more increased in the Q768A and Q768E mutant as compared to the Q768N mutant. Without being bound by theory it is speculated that the reasons for theses data is that amino acids, which can alter the binding activity of Cas9 at this specific position either by steric inhibition (alanine) or by alteration of the charge of the amino acid (glutamic acid) have a stronger effect on Cas9 specificity, whereas amino acids with a similar structure and charge as glutamine (e.g. asparagine) will have only minor effects on Cas9 binding at this specific position.

Accordingly, the herewith enclosed data clearly indicate that the specificity of Cas9 can not only be increased by substituting the positions R63 and Q768 with alanine, but that also an increased specificity can be obtained if these positions are substituted with glutamate (or, aspartate, based on the similar charge) or amino acids structural similar to alanine (valine, isoleucine, leucine). In addition, amino acids such as proline that can disrupt the structure of the Cas9 itself might, in theory, influence the overall activity of the Cas9 protein and may therefore not suitable for enhancing Cas9 specificity at these very specific sites.

For instance, the Cas9 protein of the present invention can comprises or consists of a polypeptide with an amino acid sequence according to SEQ ID NO: 1 wherein the arginine at position 63 and the glutamine at position 768 are replaced by any one of the amino acids shown below. In one aspect, the Cas9 protein of the invention comprises or consists of a polypeptide with an amino acid sequence having at least 90% identity to SEQ ID NO: 1 wherein the position corresponding to R63 of SEQ ID NO:1 and the position corresponding to Q768 of SEQ ID NO:1 are replaced by any one of the amino acids shown below, and wherein said Cas9 protein has enhanced specificity compared to a polypeptide with the amino acid sequence according to SEQ ID NO: 1.

In particular, the arginine at position 63 (or the position corresponding to R63 of SEQ ID NO:1 in a Cas9 protein having at least 90% sequence identity to SEQ ID NO: 1) may be replaced by any one of the amino acids selected from the group consisting of (wherein the above mentioned amino acids are preferred over later mentioned amino acids):

Alanine: Ala (A)

Glutamic acid: Glu (E)

Aspartic acid: Asp (D)

Glycine: Gly (G)

Valine: Val (V)

Isoleucine: Ile (I)

Leucine: Leu (L)

Lysine: Lys (K)

Asparagine: Asn (N)

Glutamine: Gln (Q)

Serine: Ser (S)

Threonine: Thr (T)

Histidine: His (H)

Methionine: Met (M)

Phenylalanine: Phe (F)

Cysteine: Cys (C)

Tryptophan: Trp (W)

Tyrosine: Tyr (Y)

Proline: Pro (P)

and/or the glutamine at position 768 (or the position corresponding to Q768 of SEQ ID NO:1 in a Cas9 protein having at least 90% sequence identity to SEQ ID NO: 1) may be replaced by any one of the amino acids selected from the group consisting of (wherein the above mentioned amino acids are preferred over later mentioned amino acids):

Alanine: Ala (A)

Glutamic acid: Glu (E)

Aspartic acid: Asp (D)

Glycine: Gly (G)

Valine: Val (V)

Isoleucine: Ile (I)

Leucine: Leu (L)

Arginine: Arg (R)

Lysine: Lys (K)

Asparagine: Asn (N)

Serine: Ser (S)

Threonine: Thr (T)

Histidine: His (H)

Methionine: Met (M)

Phenylalanine: Phe (F)

Cysteine: Cys (C)

Tryptophan: Trp (W)

Tyrosine: Tyr (Y)

Proline: Pro (P)

As mentioned, in the above list the above mentioned amino acids are preferred over later mentioned amino acids. Thus, substitution of R63 and/or Q768 (e.g. R63 and Q768) with alanine is more preferred than substitution of R63 and/or Q768 (e.g. R63 and Q768) with glutamic acid and so on.

For instance, the Cas9 protein can comprise or consist of a polypeptide with an amino acid sequence according to SEQ ID NO: 1 wherein the arginine at position 63 and the glutamine at position 768 are replaced by Ala.

For instance, the Cas9 protein can comprise or consist of a polypeptide with an amino acid sequence according to SEQ ID NO: 1 wherein the arginine at position 63 and the glutamine at position 768 are replaced by Glu.

For instance, the Cas9 protein can comprise or consist of a polypeptide with an amino acid sequence according to SEQ ID NO: 1 wherein the arginine at position 63 and the glutamine at position 768 are replaced by Asp.

For instance, the Cas9 protein can comprise or consist of a polypeptide with an amino acid sequence according to SEQ ID NO: 1 wherein the arginine at position 63 and the glutamine at position 768 are replaced by Gly.

For instance, the Cas9 protein can comprise or consist of a polypeptide with an amino acid sequence according to SEQ ID NO: 1 wherein the arginine at position 63 and the glutamine at position 768 are replaced by Val.

For instance, the Cas9 protein can comprise or consist of a polypeptide with an amino acid sequence according to SEQ ID NO: 1 wherein the arginine at position 63 and the glutamine at position 768 are replaced by Ile.

For instance, the Cas9 protein can comprise or consist of a polypeptide with an amino acid sequence according to SEQ ID NO: 1 wherein the arginine at position 63 and the glutamine at position 768 are replaced by Leu.

For instance, the Cas9 protein can comprise or consist of a polypeptide with an amino acid sequence according to SEQ ID NO: 1 wherein the arginine at position 63 is replaced by any residue mentioned above (e.g. by Ala, Glu or Asp) and the glutamine at position 768 is replaced by Arg.

For instance, the Cas9 protein can comprise or consist of a polypeptide with an amino acid sequence according to SEQ ID NO: 1 wherein the arginine at position 63 and the glutamine at position 768 are replaced by Lys.

For instance, the Cas9 protein can comprise or consist of a polypeptide with an amino acid sequence according to SEQ ID NO: 1 wherein the arginine at position 63 and the glutamine at position 768 are replaced by Asn.

For instance, the Cas9 protein can comprise or consist of a polypeptide with an amino acid sequence according to SEQ ID NO: 1 wherein the arginine at position 63 is replaced by Gln and the glutamine at position 768 is replaced by any residue mentioned above (e.g. by Ala, Glu or Asp).

For instance, the Cas9 protein can comprise or consist of a polypeptide with an amino acid sequence according to SEQ ID NO: 1 wherein the arginine at position 63 and the glutamine at position 768 are replaced by Ser.

For instance, the Cas9 protein can comprise or consist of a polypeptide with an amino acid sequence according to SEQ ID NO: 1 wherein the arginine at position 63 and the glutamine at position 768 are replaced by Thr.

For instance, the Cas9 protein can comprise or consist of a polypeptide with an amino acid sequence according to SEQ ID NO: 1 wherein the arginine at position 63 and the glutamine at position 768 are replaced by His.

For instance, the Cas9 protein can comprise or consist of a polypeptide with an amino acid sequence according to SEQ ID NO: 1 wherein the arginine at position 63 and the glutamine at position 768 are replaced by Met.

For instance, the Cas9 protein can comprise or consist of a polypeptide with an amino acid sequence according to SEQ ID NO: 1 wherein the arginine at position 63 and the glutamine at position 768 are replaced by Phe.

For instance, the Cas9 protein can comprise or consist of a polypeptide with an amino acid sequence according to SEQ ID NO: 1 wherein the arginine at position 63 and the glutamine at position 768 are replaced by Cys.

For instance, the Cas9 protein can comprise or consist of a polypeptide with an amino acid sequence according to SEQ ID NO: 1 wherein the arginine at position 63 and the glutamine at position 768 are replaced by Trp.

For instance, the Cas9 protein can comprise or consist of a polypeptide with an amino acid sequence according to SEQ ID NO: 1 wherein the arginine at position 63 and the glutamine at position 768 are replaced by Tyr.

For instance, the Cas9 protein can comprise or consist of a polypeptide with an amino acid sequence according to SEQ ID NO: 1 wherein the arginine at position 63 and the glutamine at position 768 are replaced by Pro.

Any combination of different amino acids is also envisaged herein. For example, the arginine at position 63 (or the position corresponding to R63 of SEQ ID NO:1 in a Cas9 protein having at least 90% sequence identity to SEQ ID NO: 1) may be replaced by one of the amino acids mentioned above (e.g. by A, E, D, G, V, I, L, K, N, Q, S, T, H, M, F, C, W, Y, or P, wherein the first mentioned amino acids are preferred over later mentioned amino acids) and the glutamine at position 768 (or the position corresponding to Q768 of SEQ ID NO:1 in a Cas9 protein having at least 90% sequence identity to SEQ ID NO: 1) may be replaced independently by one of the amino acids mentioned above (e.g. A, E, D, G, V, I, L, R, K, N, S, T, H, M, F, C, W, Y, or P, wherein the first mentioned amino acids are preferred over later mentioned amino acids). For example, the arginine at position 63 (or the position corresponding to R63 of SEQ ID NO:1 in a Cas9 protein having at least 90% sequence identity to SEQ ID NO: 1) may be replaced by alanine and the glutamine at position 768 (or the position corresponding to Q768 in a Cas9 protein having at least 90% sequence identity to SEQ ID NO: 1) may be replaced by glycine etc. Any combination with the amino acids disclosed above is envisaged herein. In accordance with the definition above, any of the above mentioned Cas9 proteins has enhanced specificity compared to a polypeptide with the amino acid sequence according to SEQ ID NO: 1, i.e. the wild type Cas9. Furthermore, any of the above-defined %-sequence identity is also applicable to those Cas9 proteins.

Furthermore, the Cas9 protein of the present invention can have additional useful mutations. Such mutations include mutations which decrease the Cas9 nuclease activity. Decreased nuclease activity means that only one strand of the DNA at the target sequence/site is cleaved by the Cas9 (nickase). Decreased nuclease activity can also mean that the nuclease activity is completely absent/lost, i.e. that Cas9 does not cleave any of the DNA strands at the target sequence/site (which is known in the art as, e.g., dead-Cas9 or dCas9). Specifically, the Cas9 protein of the present invention can further comprise the D10A or D10N mutation. Thus, the Cas9 protein of the present invention can further comprise the D10A mutation. Thus, the Cas9 protein of the present invention can further comprise the D10N mutation. Alternatively, the Cas9 protein of the present invention can further comprise the H840A H840N or N840Y mutation. Thus, the Cas9 protein of the present invention can further comprise the H840A mutation. Thus, the Cas9 protein of the present invention can further comprise the H840N mutation. Thus, the Cas9 protein of the present invention can further comprise the N840Y mutation. Any combination of said mutations is also envisaged herein. For instance, the Cas9 protein of the present invention can further comprise the D10A mutation and the H840A mutation. For instance, the Cas9 protein of the present invention can further comprise the D10A mutation and the H840N mutation. For instance, the Cas9 protein of the present invention can further comprise the D10A mutation and the N840Y mutation. For instance, the Cas9 protein of the present invention can further comprise the D10N mutation and the H840A mutation. For instance, the Cas9 protein of the present invention can further comprise the D10N mutation and the H840N mutation. For instance, the Cas9 protein of the present invention can further comprise the D10N mutation and the N840Y mutation. Thus, in addition to the mutations at positions R63 and Q768 of SEQ ID NO: 1 (or at the positions corresponding R63 and Q768 in a Cas9 protein having at least 90% sequence identity to SEQ ID NO: 1) the Cas9 protein of the invention may comprise further mutation which decrease or abolish the nuclease activity. Several applications are known for Cas9 proteins having a decreased or absent nuclease activity (Adli, Nat Commun. 2018 May 15; 9(1):1911 (PMID: 29765029)). And the possibility to link dCas9 to a base editor represents a promising strategy for site specific genome editing without the detrimental effects of double-strand breaks (Eid, Biochem J. 2018 Jun. 11; 475(11):1955-1964 (PMID: 29891532)). All these applications can also be carried out with the Cas9 protein of the present invention. The Cas9 protein of the present invention binds to its target sequence with improved specificity as compared to wild type Cas9. Therefore, a Cas9 protein of the invention which has a decreased or absent nuclease activity (i.e. a nuclease-deficient Cas9 protein according to the invention) may be used to bind to a desired site of the genome without cutting the genome. For instance, the nuclease-deficient Cas9 protein according to the invention may bind to a genomic region which regulates the transcription of a desired target gene (such as the promoter sequence). Therefore, the nuclease-deficient Cas9 protein according to the invention may be used for controlling the transcription of a desired gene. Alternatively, the nuclease-deficient Cas9 protein according to the present invention may be used for identifying a particular genomic sequence, e.g., in a diagnostic method. In this regard, the nuclease-deficient Cas9 protein according to the invention may be coupled to a reporter molecule. Suitable reporter are, e.g., green fluorescent protein (GFP), red fluorescent protein (RFP), yellow fluorescent protein (YFP), or cyan fluorescent protein (CFP).

The Cas9 protein of the present invention can further comprise one or more nuclear localization signal(s) (NLS(s)). The Cas9 protein comprises one, two, three, four, five, six, seven, eight, nine or ten NLS(s). Preferably, the Cas9 protein comprises one, two, three, four, or five NLS(s). More preferably, the Cas9 protein comprises one, two, three or four NLS(s). Even more preferably, the Cas9 protein comprises one, two or three NLS(s). More preferably, the Cas9 protein comprises one, two, three or four NLS(s). Even more preferably, the Cas9 protein comprises one or two NLS(s). When the Cas9 protein comprises NLS(s), the NLS(s) are either directly fused to the N- and/or C-terminus of the Cas9 or are located at the N- and/or C-terminus of the Cas9.

The NLSs can be located at the N-terminus of Cas9 and the C-terminus of Cas9. Alternatively, the NLS(s) are located either at the N-terminus of Cas9 or at the C-terminus of Cas9. One, two, three, four, five, six, seven, eight, nine or ten NLS(s) is/are located at the N-terminus of Cas9 and/or one, two, three, four, five, six, seven, eight, nine or ten NLS(s) is/are located at the C-terminus of Cas9.

One NLS can be located at the N-terminus of Cas9. Alternatively, one NLS can be located at the C-terminus of Cas9. Preferably, one NLS is located at the N-terminus of Cas9 and one NLS is located at the C-terminus of Cas9. Also, two NLSs can be located at the N-terminus of Cas9 and one NLS can be located at the C-terminus of Cas9. Also, one NLS can be located at the N-terminus of Cas9 and two NLSs can be located at the C-terminus of Cas9. Also, two NLSs can be located at the N-terminus of Cas9 and two NLSs can be located at the C-terminus of Cas9. Also, two NLSs can be located at the N-terminus of Cas9 and three NLSs can be located at the C-terminus of Cas9. Also, three NLSs can be located at the N-terminus of Cas9 and two NLSs can be located at the C-terminus of Cas9. Also, three NLSs can be located at the N-terminus of Cas9 and three NLSs can be located at the C-terminus of Cas9. Further combinations of NLSs at the N-terminus and/or the C-terminus of Cas9 are also envisaged herein.

The expression “located at” as used herein means that the NLS is directly at the N- or C-terminus of Cas9. Also, “located at” means that about 1, 2, 3, 4, 5, 10, 15, 20, 25, 30, 40, 50, 100, 200, 300 or 500 or more amino acids are between the N- or C-terminus of Cas9 and the NLS. Preferably, “located at” means that 1 to 200 amino acids are between the NLS and the N- or C-terminus of Cas9. More preferably, “located at” means that 1 to 100 amino acids are between the NLS and the N- or C-terminus of Cas9. Even more preferably, “located at” means that 1 to 50 amino acids are between the NLS and the N- or C-terminus of Cas9. Even more preferably, “located at” means that 1 to 10 amino acids are between the NLS and the N-o r C-terminus of Cas9.

The skilled person is well aware of NLS known in the art. Non-limiting examples of NLSs include an NLS sequence derived from: the NLS of the SV40 virus large T-antigen; the NLS from nucleoplasmin; the c-myc NLS; the hnRNPA1 M9 NLS; NLS sequences of the IBB domain from importin-alpha; NLS sequences of the myoma T protein; NLS sequence of the of human p53; NLS sequence of the mouse c-abl IV; NLS sequences of influenza virus NS1; NLS sequences of the Hepatitis virus delta antigen; NLS sequences of the mouse Mx1 protein, NLS sequences of the human poly(ADP-ribose) polymerase; NLS sequence of the steroid hormone receptors (human) glucocorticoid. The one or more NLSs are of sufficient strength to drive accumulation of the Cas9 in a detectable amount in the nucleus of a eukaryotic cell. Strength of nuclear localization activity may derive from the number of NLSs in the Cas9, the particular NLS(s) used, or a combination of these factors. Detection of accumulation in the nucleus may be performed by any suitable technique. For example, a detectable marker may be fused to the Cas9, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot or enzyme activity assay. Accumulation in the nucleus may also be determined indirectly, such as by an assay for the effect of CRISPR complex formation (e.g. assay for DNA cleavage or mutation at the target sequence, or assay for altered gene expression activity affected by CRISPR complex formation and/or Cas9 enzyme activity), as compared to a control not exposed to the Cas9 or complex, or exposed to a Cas9 lacking the one or more NLSs.

Cell-penetrating peptides are short peptides that facilitate the movement of a wide range of biomolecules across the cell membrane into the cytoplasm or other organelles, e.g. the mitochondria and the nucleus. Various cell-penetrating peptides are known in the art. The skilled person is aware of those peptides and knows how the Cas9 protein of the present invention can be modified so that it comprises cell-penetrating peptide(s). Accordingly, the Cas9 protein of the present invention can further comprise one or more cell-penetrating peptide(s) that facilitates delivery to the intracellular space, e.g., HIV-derived TAT peptide, penetratins, transportans, or hCT derived cell-penetrating peptides (see, e.g., Caron et al, Mol Ther. 2001, 3(3):310-8; Langel, Cell-Penetrating Peptides: Processes and Applications (CRC Press, Boca Raton Fla. 2002); El-Andaloussi et al, Curr Pharm Des. 2005, 11(28):3597-3611; Deshayes et al, Cell Mol Life Sci. 2005, 62(16): 1839-49). Cell-penetrating peptides that are commonly used in the art and can be included (fused to) a Cas9 protein of the present invention include TAT (Frankel et al., Cell 1988, 55:1189-1193, Vives et al., Biol. Chem. 1997, J272:16010-16017), penetratin (Derossi et al, J. Biol. Chem. 1994, 269:10444-10450), polyarginine peptide sequences (Wender et al, Proc. Natl. Acad. Sci. USA 2000, 97:13003-13008, Futaki et al., J. Biol. Chem. 2001, 276:5836-5840), and transportan (Pooga et al., Nat. Biotechnol. 1998, 16:857-861).

Preferably, the Cas9 protein of the present invention comprises one, two or three cell-penetrating peptide(s). More preferably, the Cas9 protein of the present invention comprises one or two cell-penetrating peptide(s). Most preferably, the Cas9 protein of the present invention comprises one cell-penetrating peptide.

The Cas9 protein of the present invention can further comprise one or more tags.

The Cas9 protein comprises one, two, three, four, five, six, seven, eight, nine or ten tag(s). Preferably, the Cas9 protein comprises one, two, three, four, or five tag(s). More preferably, the Cas9 protein comprises one, two, three or four tag(s). Even more preferably, the Cas9 protein comprises one, two or three tag(s). More preferably, the Cas9 protein comprises one, two, three or four tag(s). Even more preferably, the Cas9 protein comprises one or two tag(s). Most preferably, the Cas9 protein comprises one tag. When the Cas9 protein comprises tag(s), the tag(s) can either be directly fused to the N- and/or C-terminus of the Cas9 or can be located at the N- and/or C-terminus of the Cas9. The expression “located at” is used in accordance with the definition provided above.

Preferably, one tag is located at the N-terminus of Cas9. Preferably, one tag is located at the C-terminus of Cas9. Also, one tag is located at the N-terminus of Cas9 and one tag is located at the C-terminus of Cas9. Also, two tags can be located at the N-terminus of Cas9 and one tag can be located at the C-terminus of Cas9. Also, one tag can be located at the N-terminus of Cas9 and two tags can be located at the C-terminus of Cas9. Also, two tags can be located at the N-terminus of Cas9 and two tags can be located at the C-terminus of Cas9. Also, two tags can be located at the N-terminus of Cas9 and three tags can be located at the C-terminus of Cas9. Also, three tags can be located at the N-terminus of Cas9 and two tags can be located at the C-terminus of Cas9. Also, three tags can be located at the N-terminus of Cas9 and three tags can be located at the C-terminus of Cas9. Further combinations of tags at the N-terminus and/or the C-terminus of Cas9 are also envisaged herein.

(Protein) tags are peptide sequences genetically grafted onto a recombinant protein. Such tags are often removable by chemical agents or by enzymatic means (e.g. proteolysis or intein splicing). In general, tags are attached to proteins for various purposes. For instance, affinity tags are appended to proteins so that they can be purified from their crude biological source using an affinity technique. Affinity tags are chitin binding protein (CBP), maltose binding protein (MBP), Strep-tag or glutathione-S-transferase (GST). Furthermore, the poly(His) tag (or His-tag) is known which binds to metal matrices. Also, solubilization tags can be used. Such solubilization tags can be used for recombinant proteins expressed in e.g. E. coli in order to assist in the proper folding of proteins and in order to keep these proteins from precipitating (thioredoxin (TRX) and poly(NANP)). Also known are chromatography tags which can be used to alter chromatographic properties of the protein to afford different resolution across a particular separation technique. Such tags can consist of polyanionic amino acids (e.g. FLAG-tag). Also known are epitope tags which are short peptide sequences which are chosen because high-affinity antibodies can be reliably produced in many different species. These are usually derived from viral genes, which explain their high immunoreactivity (e.g. V5-tag, Myc-tag, HA-tag and NE-tag). These tags can be used in western blotting, immunofluorescence and immunoprecipitation experiments, and can also be used in antibody purification. Also known are fluorescence tags which are generally used to give a visual readout on a protein. Green fluorescence protein (GFP) and its variants are the most commonly used fluorescence tags. Tags can be removed by specific proteolysis (e.g. by TEV protease, Thrombin, Factor Xa or Enteropeptidase). The above-described tags can be used in the present invention. Specifically, the Cas9 protein of the present invention can comprise said tags.

The Cas9 protein of the present invention can comprise one or more of the following tags: AviTag, Calmodulin-tag, polyglutamate tag, E-tag, FLAG-tag, HA-tag, (poly)His-tag, Myc-tag, NE-tag, S-tag, SBP-tag, Softag 1, Softag 3, Strep-tag, TC tag, Ty tag, V5 tag, VSV-tag, Xpress tag, Isopeptag, SpyTag, SnoopTag, BCCP, Glutathione-S-transferase-tag, GFP-tag, HaloTag, Maltose binding protein-tag, Nus-tag, Thioredoxin-tag, Fc-tag.

Preferably, the Cas9 protein comprises one or more of the poly(His) tag, GFP, Flag-tag, Myc-tag, HA-tag. Preferably, the Cas9 protein comprises the poly(His) tag. Preferably, the Cas9 protein comprises the Flag-tag. Preferably, the Cas9 protein comprises the poly(His) tag. Preferably, the Cas9 protein comprises the Myc-tag. Preferably, the Cas9 protein comprises the poly(His) tag. Preferably, the Cas9 protein comprises the HA-tag. More preferably, the Cas9 protein comprises the GFP tag.

Also provided herein are fusion proteins comprising the Cas9 protein of the present invention fused to a heterologous functional domain, with an optional intervening linker, wherein the linker does not interfere with activity of the fusion protein. The linkers are short, e.g., 2 to 20 amino acids, and are typically flexible (i.e., comprising amino acids with a high degree of freedom such as glycine, alanine, and serine). The heterologous functional domain can act on DNA or protein, e.g., on chromatin. The heterologous functional domain can be a transcriptional activation domain. The transcriptional activation domain can be selected from VP64 or NF-κB p65. The heterologous functional domain can be a transcriptional silencer or transcriptional repression domain. The transcriptional repression domain can be a Kruppel-associated box (KRAB) domain, ERF repressor domain (ERD), or mSin3A interaction domain (SID). The transcriptional silencer can be Heterochromatin Protein 1 (HP1), e.g., HP1α or HP1β. The heterologous functional domain can be an enzyme that modifies the methylation state of DNA. The enzyme that modifies the methylation state of DNA is a DNA methyltransferase (DNMT) or the entirety or the dioxygenase domain of a TET protein, e.g., a catalytic module comprising the cysteine-rich extension and the 20GFeDO domain encoded by 7 highly conserved exons, e.g., the Tet1 catalytic domain comprising amino acids 1580-2052, Tet2 comprising amino acids 1290-1905 and Tet3 comprising amino acids 966-1678. The TET protein or TET-derived dioxygenase domain can be from TET1. The heterologous functional domain can be an enzyme that modifies a histone subunit. The enzyme that modifies a histone subunit can be a histone acetyftransferase (HAT), histone deacetylase (HDAC), histone methyltransferase (HMT) or histone demethylase. The heterologous functional domain can be a biological tether. The biological tether can be MS2, Csy4 or lambda N protein. The heterologous functional domain can be FokI.

Fusion provided herein also encompass the Cas9 protein of the present invention fused to one or more anti-CRISPR (Acr) polypeptide(s)/protein(s). The Arc can be selected from one or more of AcrF1, AcrF2, AcrF3, AcrF4, AcrF5, AcrE1, AcrE2, AcrE3, AcrE4, Aca1, Aca2, AcrF6, AcrF7, AcrF8, AcrF9, AcrF10, AcrIIC1, AcrIIC2, AcrIIC3, AcrIIA1, AcrIIA2, AcrIIA3 and AcrIIA4. The skilled person knows the Arc polypeptides/proteins, e.g., from Pawluk et al., Nature Reviews Microbiology (2018), 16: 12-17.

Nucleic Acids, Vectors, Promoters, Host Cells, Expression Systems and Methods for Producing the Cas9 Protein of the Present Invention

Also provided herein is a polynucleotide which encodes the Cas9 protein of the present invention. Thus, the present invention also encompasses a polynucleotide which encodes the Cas9 protein of the invention.

Herein, the term “polynucleotide” refers to nucleic acids such as DNA, such as cDNA or genomic DNA, and RNA. The term “polynucleotide” can be exchanged by, e.g., the term “nucleic acid” or “nucleotide sequence”. The polynucleotides used in accordance with the present invention may be of natural as well as of (semi) synthetic origin. Thus, the polynucleotides may, for example, be nucleic acid molecules that have been synthesized according to conventional protocols of organic chemistry. The person skilled in the art is familiar with the preparation and the use of polynucleotides (see, e.g., Sambrook and Russel “Molecular Cloning, A Laboratory Manual”, Cold Spring Harbor Laboratory, N.Y. (2001)). The polynucleotides used in accordance with the invention may comprise or consist of nucleic acid mimicking molecules known in the art. They may contain additional non-natural or derivatized nucleotide bases, as will be readily appreciated by those skilled in the art. Nucleic acid mimicking molecules or nucleic acid derivatives according to the invention include, without being limiting, phosphorothioate nucleic acid, phosphoramidate nucleic acid, morpholino nucleic acid, hexitol nucleic acid (HNA), peptide nucleic acid (PNA) and locked nucleic acid (LNA).

The polynucleotide encoding the Cas9 protein of the present invention can be isolated.

The polynucleotide encoding the Cas9 protein of the present invention can be recombinant.

Any of the Cas9 proteins of the present invention can be encoded by several different polynucleotides/nucleic acids. This is due to the degenerative of the genetic code meaning that a certain amino acid can be encoded by several different nucleotide triplets. The skilled person is well aware of the degenerative of the genetic code.

The polynucleotide encoding the Cas9 protein of the present invention can be codon-optimized for expression in eukaryotic cells.

An example of a codon-optimized sequence is a sequence optimized for expression in a eukaryote, e.g., humans (i.e. being optimized for expression in humans), or for another eukaryote, animal or mammal as herein discussed; see, e.g., SaCas9 human codon optimized sequence in WO 2014/093622. Human codon-optimized SpCas9 is described, e.g., in Hsu et al., Nature Biotechnology 31, 827-832 (2013). Whilst this is preferred, it will be appreciated that other examples are possible and codon-optimization for a host species other than human or for codon-optimization for specific organs is known. The codon-optimized sequence for expression in particular cells, such as eukaryotic cells may be those of or derived from a particular organism, such as a mammal, including but not limited to human, or non-human eukaryote or animal or mammal as herein discussed, e.g., mouse, rat, rabbit, dog, livestock, or non-human mammal or primate. Codon-optimization refers to a process of modifying a nucleic acid sequence for enhanced expression in the host cells of interest by replacing at least one codon (e.g. about or more than about 1, 2, 3, 4, 5, 10, 15, 20, 25, 50 or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon-optimization. Codon usage tables are readily available, for example, at the “Codon Usage Database” available at www.kazusa.orjp/codon/ and these tables can be adapted in a number of ways (see Nakamura, et al. “Codon usage tabulated from the international DNA sequence databases: status for the year 2000”, Nucl, Acids Res 2000, 28:292). Computer algorithms for codon-optimizing a sequence for expression in a particular host cell are also available; see, e.g., Gene Forge (Aptagen; Jacobus, Pa.).

The polynucleotide encoding the Cas9 protein of the present invention can be present in a vector. Thus, the present invention is also directed to a vector comprising the polynucleotide encoding the Cas9 protein of the invention.

A (expression) vector must have elements necessary for gene expression. These may include a promoter, the correct translation initiation sequence such as a ribosomal binding site, a start codon, a termination codon and a transcription termination sequence. The expression vectors must have the elements for expression that is appropriate for the chosen host since differences in the protein synthesis machinery exist between prokaryotes and eukaryotes. For instance, prokaryotes expression vectors would have a Shine-Dalgarno sequence while eukaryotes expression vectors contain the so-called Kozak (consensus) sequence.

Examples of the vectors include M13 vectors, pUC vectors, pBR322, pBluescript, and pCR-Script. Alternatively, when aiming to subclone and excise cDNA, in addition to the vectors described above, pGEM-T, pDIRECT, pT7, and such can be used. Expression vectors are particularly useful when using vectors for producing the polypeptides of the present invention. For example, when a host cell is E. coli such as JM109, DH5α, HB101, and XL1-Blue, the expression vectors must carry a promoter that allows efficient expression in E. coli, for example, lacZ promoter (Ward et al., Nature (1989) 341: 544-546; FASEB J. (1992) 6: 2422-2427; its entirety are incorporated herein by reference), araB promoter (Better et al., Science (1988) 240: 1041-1043), T7 promoter, or such. Such vectors include pGEX-5X-1 (Pharmacia), “QIAexpress system” (Qiagen), pEGFP, or pET (in this case, the host is preferably BL21 that expresses T7 RNA polymerase) in addition to the vectors described above. The vectors may contain signal sequences for polypeptide secretion. As a signal sequence for polypeptide secretion, a pelB signal sequence (Lei, S. P. et al J. Bacteriol. (1987) 169: 4379) may be used when a polypeptide is secreted into the E. coli periplasm. The vector can be introduced into host cells by lipofectin method, calcium phosphate method, and DEAE-Dextran method. The vectors of the present invention also include mammalian expression vectors (for example pcDNA3 (Invitrogen), pEGF-BOS (Nucleic Acids. Res. 1990, 18(17): p5322), pEF, and pCDM8), insect cell-derived expression vectors (for example, the “Bac-to-BAC baculovirus expression system” (Gibco-BRL) and pBacPAK8), plant-derived expression vectors (for example, pMH1 and pMH2), animal virus-derived expression vectors (for example, pHSV, pMV, and pAdexLcw), retroviral expression vectors (for example, pZIPneo), yeast expression vectors (for example, “Pichia Expression Kit” (Invitrogen), pNV11, and SP-Q01), and Bacillus subtilis expression vectors (for example, pPL608 and pKTH50). The type of vector can be appropriately selected by those skilled in the art depending on the host cells to be introduced with the vector.

Vectors which can be used herein can be obtained, e.g., from http://www.addgene.org.

The vectors used herein can have a gene for selecting transformed cells (for example, a drug resistance gene that allows evaluation using an agent (neomycin, G418 etc.)). Non-limiting examples of such vectors include pMAM, pDR2, pBK-RSV, pBK-CMV, pOPRSV, and pOP13.

Examples of mammalian expression vectors include adenoviral vectors, the pSV and the pCMV series of plasmid vectors, vaccinia and retroviral vectors, and also baculovirus.

When inserting a polynucleotide (i.e. DNA) encoding the Cas9 of the present invention into an (expression) vector, the polynucleotide (i.e. the DNA) is preferably inserted into a suitable vector so that the Cas9 is expressed under the control of/operably linked to a transcription regulatory element (expression-regulating region), such as an enhancer or promoter. Accordingly, the transcription regulatory element is preferably a promoter. The transcription regulatory element used herein can also be an enhancer. The transcription regulatory element used herein can also be a promoter and an enhancer. In the vector used herein, the polynucleotide is preferably under the control of/operably linked to a promoter. In the vector, the polynucleotide is preferably under the control of/operably linked to an enhancer. One or more promoter(s) and/or enhancer(s) can be used. For instance, in the vector, the polynucleotide is preferably under the control of/operably linked to one promoter. In the vector, the polynucleotide can also be under the control of/operably linked to two promoters. In the vector, the polynucleotide is preferably under the control of/operably linked to one enhancer. In the vector, the polynucleotide can also be under the control of/operably linked to two enhancers.

The expression “operably linked” is intended to mean that the polynucleotide/nucleotide sequence of interest is linked to the transcription regulatory element(s) in a manner that allows for expression of the polynucleotide/nucleotide sequence (e.g. in an in vitro transcription/translation system or in a host cell when the vector is introduced into the host cell).

When aiming for expression in animal cells such as, e.g., CHO, COS and NIH3T3 cells, the vectors must have a promoter essential for expression in cells, e.g., SV40 promoter (Mulligan et al., Nature (1979) 277: 108), MMTV-LTR promoter, EF1 alpha promoter (Mizushima et al., Nucleic Acids Res. (1990) 18: 5322), CAG promoter (Gene. (1990) 18:5322) and CMV promoter. Multiple further promoters which can be used in accordance with the present invention are known in the art.

The promoter initiates the transcription. Therefore, it is the point of control for the expression of the gene (i.e. the polynucleotide encoding the Cas9 protein of the present invention). The promoters used in expression vector can be inducible, i.e. the protein synthesis is only initiated when required by the introduction of an inducer, e.g. IPTG. Gene expression however can also be constitutive (i.e. the protein (the Cas9 protein) is constantly expressed).

Enhancer(s), as used herein, refers to a short (50 to 1500 bases) region of DNA which can be bound by proteins (e.g. activators) to increase (the likelihood that) transcription of a particular gene (e.g. the polynucleotide encoding the Cas9 protein of the present invention). These proteins are usually referred to as transcription factors. Enhancers are cis-acting. They can be located up to 1 Mega bases (1,000,000 bases) away from the gene. They can be located upstream or downstream from the gene of interest. The skilled person is aware of multiple enhancer which can be used in accordance with the present invention. For instance, HACNS1 (also known as CENTG2 and located in the Human Accelerated Region 2) is a gene enhancer which can be used herein.

Furthermore, host cells can be transformed/transfected with the (expression) vector(s) which encode/express the Cas9 protein of the present invention. Thereby, host cell(s) is/are obtained which comprise/encompass/encode/express the Cas9 protein of the present invention. In such cases, an appropriate combination of host and expression vector may be used. The skilled person is well aware of methods in the art which can be used for transformation/transfection in order to generate host cells comprising the Cas9 protein of the present invention and/or the polynucleotide encoding the Cas9 protein and/or vector(s) which comprise the polynucleotide encoding the Cas9 protein. For instance, Lipofectamine® 2000 can be used for transfection. Also, the skilled person knows that transient or stable transfection can be used. For stable transfection, antibiotic resistance genes (e.g. G418) can be used for selectine the cells which are stably transformed/transfected with the vector(s) of interest. Accordingly, the present invention is directed to a host cell comprising the Cas9 protein of the present invention. Also, the present invention is directed to a host cell comprising the polynucleotide encoding the Cas9 protein of the present invention. Also, the present invention is directed to a host cell comprising the vector comprising the polynucleotide encoding the Cas9 protein of the present invention. Appropriate host cells can be selected by those skilled in the art and are known. Cultured mammalian cell lines such as the Chinese hamster ovary (CHO), COS, including human cell lines such as HEK and HeLa cells can be used as the host cell(s) and can also be used to produce the Cas9 protein.

In addition, the following method can be used exemplarily for stable gene expression and gene copy number amplification in cells: CHO cells deficient in a nucleic acid synthesis pathway are introduced with a vector that carries a DHFR gene which compensates for the deficiency (for example, pCHOI), and the vector is amplified using methotrexate (MTX). Alternatively, the following method can be used exemplarily for transient gene expression: COS cells with a gene expressing SV40 T antigen on their chromosome are transformed with a vector with an SV40 replication origin (pcD and such). Replication origins derived from polyoma virus, adenovirus, bovine papilloma virus (BPV), and such can also be used. To amplify gene copy number in host cells, the expression vectors may further carry selection markers such as aminoglycoside transferase (APH) gene, thymidine kinase (TK) gene, E. coli xanthine-guanine phosphoribosyltransferase (Ecogpt) gene and dihydrofolate reductase (dhfr) gene.

The Cas9 of the present invention can be collected, for example, by culturing transformed/transfected cells, and then separating the Cas9 from the inside of the transformed/transfected cells or from the culture media. SpCas9 can be separated and purified using an appropriate combination of methods such as centrifugation, ammonium sulfate fractionation, salting out, ultrafiltration, 1 q, FcRn, protein A, protein G column, affinity chromatography, ion exchange chromatography, and gel filtration chromatography.

A method for producing the Cas9 of the present invention can comprise the steps of:

(a) altering the polynucleotide/nucleic acid encoding the wild type SpCas9 in order to obtain a polynucleotide/nucleic acid which encodes the Cas9 protein of the present invention;

(b) introducing the polynucleotide/nucleic acid into (a) suitable host cell(s);

(c) culturing said host cell(s) to induce expression of the Cas9 of the present invention; and

(d) collecting the Cas9 of the present invention from the host cell culture.

A method for producing the Cas9 of the present invention can comprise the steps of:

(a) introducing the polynucleotide/nucleic acid encoding the Cas9 protein of the present invention into (a) suitable host cell(s);

(b) culturing said host cell(s) to induce expression of the Cas9 of the present invention; and

(c) collecting the Cas9 of the present invention from the host cell culture.

In the above-described methods for production, the polynucleotide/nucleic acid encoding the SpCas9 is altered as desired, i.e. the polynucleotide/nucleic acid encoding the SpCas9 is altered so that the polynucleotide/nucleic acid encoding the SpCas9 with the amino acid alterations in accordance with the present invention is obtained.

The present invention also encompasses such a method of production.

Pharmaceutical Compositions

The present invention provides pharmaceutical compositions comprising the Cas9 protein of the present invention. For instance, the pharmaceutical composition can comprise the Cas9 protein of the present invention and a guide RNA. The guide RNA can be a single guide RNA or a tracrRNA:crRNA duplex. Also, for instance, the pharmaceutical composition can comprise

(i) a guide RNA and the Cas9 protein according to the present invention;

(ii) a guide RNA and the polynucleotide according to the present invention; and/or

(iii) a guide RNA and the vector according to the present invention.

The pharmaceutical compositions can be formulated with pharmaceutically acceptable carriers by known methods. For example, the compositions can be used parenterally in a sterile solution or suspension for injection using water or any other pharmaceutically acceptable liquid(s). For example, the compositions can be formulated by appropriately combining the ingredients (e.g. Cas9 of the present invention and single guide RNA) with pharmaceutically acceptable carriers or media, specifically, sterile water or physiological saline, vegetable oils, emulsifiers, suspending agents, surfactants, stabilizers, flavoring agents, excipients, vehicles, preservatives, binding agents, and such, by mixing them at a unit dose and form required by generally accepted pharmaceutical implementations. Specific examples of the carriers include light anhydrous silicic acid, lactose, crystalline cellulose, mannitol, starch, carmellose calcium, carmellose sodium, hydroxypropyl cellulose, hydroxypropyl methylcellulose, polyvinylacetal diethylaminoacetate, polyvinylpyrrolidone, gelatin, medium-chain triglyceride, polyoxyethylene hardened castor oil 60, saccharose, carboxymethyl cellulose, corn starch, inorganic salt, and such. The content of the active ingredient in such a formulation is adjusted so that an appropriate dose within the required range can be obtained.

Sterile compositions for injection can be formulated using vehicles such as distilled water for injection, according to standard protocols. Aqueous solutions used for injection include, for example, physiological saline and isotonic solutions containing glucose or other adjuvants such as D-sorbitol, D-mannose, D-mannitol, and sodium chloride. These can be used in conjunction with suitable solubilizers such as alcohol, specifically ethanol, polyalcohols such as propylene glycol and polyethylene glycol, and non-ionic surfactants such as Polysorbate 80™ and HCO-50. Oils include sesame oils and soybean oils, and can be combined with solubilizers such as benzyl benzoate or benzyl alcohol. These may also be formulated with buffers, for example, phosphate buffers or sodium acetate buffers; analgesics, for example, procaine hydrochloride; stabilizers, for example, benzyl alcohol or phenol; or antioxidants. The prepared injections are typically aliquoted into appropriate ampules.

The pharmaceutical composition may optionally comprise one or more pharmaceutically acceptable excipients, such as carriers, diluents, fillers, disintegrants, lubricating agents, binders, colorants, pigments, stabilizers, preservatives, antioxidants, or solubility enhancers. Also, the pharmaceutical compositions may comprise one or more solubility enhancers, such as, e.g., poly(ethylene glycol), including poly(ethylene glycol) having a molecular weight in the range of about 200 to about 5,000 Da, ethylene glycol, propylene glycol, non-ionic surfactants, tyloxapol, polysorbate 80, macrogol-15-hydroxystearate, phospholipids, lecithin, dimyristoyl phosphatidylcholine, dipalmitoyl phosphatidylcholine, di stearoyl phosphatidylcholine, cyclodextrins, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, carboxyalkyl thioethers, hydroxypropyl methylcellulose, hydroxypropylcellulose, polyvinylpyrrolidone, vinyl acetate copolymers, vinyl pyrrolidone, sodium lauryl sulfate, dioctyl sodium sulfosuccinate, or any combination thereof.

The pharmaceutical compositions are not limited to the means and methods described herein. The skilled person can use his/her knowledge available in the art in order to construct a suitable composition. Specifically, the pharmaceutical compositions can be formulated by techniques known to the person skilled in the art such as the techniques published in Remington's Pharmaceutical Sciences, 20^{th} Edition.

The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems. In combination with a medical device is may be surgically inserted in the body. This mesial device may be but is not limited to a stent.

The pharmaceutical compositions can administered in any pharmaceutical form for oral (e.g. solid, semi-solid, liquid), dermal (e.g. dermal patch), sublingual, parenteral (e.g. injection), ophthalmic (e.g. eye drops, gel or ointment) or rectal (e.g. suppository) administration. Preferably, the composition is formulated as a tablet, capsule, suppository, dermal patch or sublingual formulation.

The pharmaceutical compositions can be administered with a single dose or with 2, 3, 4, 5, 6, 7, 8, 9, or 10 doses, if desired. The composition can be administered 1, 2, 3, 4, 5, 6, 7, 8, 9 or 10 times per day.

The pharmaceutical compositions can be administered in a dose range varying depending on the patient's body weight, age, gender, health condition, diet, administration time, administration method, excretion rate and disease severity. The pharmaceutical compositions can be administered to the patient and/or subject at a suitable dose. The dosage regiment will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. Generally, the regimen as a regular administration of the pharmaceutical composition comprising the herein defined should be, e.g., in a range as described below. Progress can be monitored by periodic assessment.

Furthermore, the method and route of administration can be appropriately selected according to the age and symptoms of the patient. A single dosage of the pharmaceutical composition can be selected, for example, from the range of 0.0001 to 1,000 mg per kg of body weight. Alternatively, the dosage may be, for example, in the range of 0.001 to 100,000 mg/patient. However, the dosage is not limited to these values. The dosage and method of administration vary depending on the patient's body weight, age, and symptoms, and can be appropriately selected by those skilled in the art.

The amount/concentration of the pharmaceutical composition as used herein can be administered at the first day of administration in a higher dose (concentration/amount) compared to the administration of the pharmaceutical composition at the following days(s) of administration (maintenance administration/maintenance dose of administration). Alternatively such decreased dose (maintenance dose) can be started after 2, 3, 4, 5, 6, 7, 8, 9 or 10 days of initial administration of the higher dose.

The present invention also provides a method of treatment wherein the pharmaceutical composition as described above is administered to a subject or patient.

The subject or patient, such as the subject in need of treatment or prevention, may be an animal (e.g., a non-human animal), a vertebrate animal, a mammal, a rodent (e.g., a guinea pig, a hamster, a rat, a mouse), a murine (e.g., a mouse), a canine (e.g., a dog), a feline (e.g., a cat), an equine (e.g., a horse), a primate, a simian (e.g., a monkey or ape), a monkey (e.g., a marmoset, a baboon), an ape (e.g., a gorilla, chimpanzee, orang-utan, gibbon), or a human. The meaning of the terms “eukaryote”, “animal”, “mammal”, etc. is well known in the art and can, for example, be deduced from Wehner and Gehring (1995; Thieme Verlag). In the context of this invention, it is also envisaged that animals are to be treated which are economically, agronomically or scientifically important. Scientifically important organisms include, but are not limited to, mice, rats, and rabbits. Non-limiting examples of agronomically important animals are sheep, cattle and pigs, while, for example, cats and dogs may be considered as economically important animals. Preferably, the subject/patient is a mammal; more preferably, the subject/patient is a human or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orang-utan, a gibbon, a sheep, cattle, or a pig); most preferably, the subject/patient is a human.

The compositions encompassing Cas9 of the present invention can also be for use in treating a genetic disorder, particularly for treating a disease which is based on one or more mutation(s) in the genome. Thus, the present invention relates to the composition of the invention for use in treating a disease which is based on one or more mutation(s). Said disease is preferably based on one mutation in the genome. Said disease may be an inheritable disease. The term “inheritable disease” is commonly known in the art and refers to a disease which can be inherited from the mother or father to the child (i.e. a disease which is transmissible from the parents to their offspring). Accordingly, the compositions are for use in treating one or more of the diseases selected from the group consisting of achondroplasia, alpha-1 antitrypsin deficiency, Alzheimer's disease, antiphospholipid syndrome, autism, autosomal dominant polycystic kidney disease, breast cancer, cancer, Charcot-Marie-Tooth, colon cancer, cri du chat, Crohn's disease, cystic fibrosis, dercum disease, down syndrome, duane syndrome, duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan Syndrome, osteogenesis imperfecta, Parkinson's disease, phenylketonuria, Poland anomaly, porphyria, progeria, prostate cancer, retinitis pigmentosa, severe combined immunodeficiency, sickle cell disease, skin cancer, spinal, muscular atrophy, Tay-Sachs, thalassemia, trimethylaminuria, Turner syndrome, velocardiofacial syndrome, Wilms-Tumour-Aniridia-Syndrom (WAGR) or Wilson disease.

There are ongoing clinical studies wherein a CRISPR complex is used for the treatment of sickle cell disease, leber's congenital amaurosis type 10, or β-thalassemia. Therefore, in a preferred aspect of the present invention the disease to be treated is any one of sickle cell disease, leber's congenital amaurosis type 10, or β-thalassemia.

The composition encompassing Cas9 of the present invention may also be used for the treatment or prevention of an infection with the human immunodeficiency virus (HIV).

Preferably, the compositions of the present invention are for use in treating Huntington's disease. Preferably, the compositions of the present invention are for use in treating Alzheimer's disease. Preferably, the compositions of the present invention are for use in treating cancer.

The compositions encompassing Cas9 of the present invention can also be for use in treating another disease, including, but not limited to, one or more of the following diseases: rheumatoid arthritis, autoimmune hepatitis, autoimmune thyroiditis, autoimmune blistering diseases, autoimmune adrenocortical disease, autoimmune hemolytic anemia, autoimmune thrombocytopenic purpura, megalocytic anemia, autoimmune atrophic gastritis, autoimmune neutropenia, autoimmune orchitis, autoimmune encephalomyelitis, autoimmune receptor disease, autoimmune infertility, chronic active hepatitis, glomerulonephritis, interstitial pulmonary fibrosis, multiple sclerosis, Paget's disease, osteoporosis, multiple myeloma, uveitis, acute and chronic spondylitis, gouty arthritis, inflammatory bowel disease, adult respiratory distress syndrome (ARDS), psoriasis, Crohn's disease, Basedow's disease, juvenile diabetes, Addison's disease, myasthenia gravis, lens-induced uveitis, systemic lupus erythematosus, allergic rhinitis, allergic dermatitis, ulcerative colitis, hypersensitivity, muscle degeneration, cachexia, systemic scleroderma, localized scleroderma, Sjogren's syndrome, Behchet's disease, Reiter's syndrome, type I and type II diabetes, bone resorption disorder, graft-versus-host reaction, ischemia-reperfusion injury, atherosclerosis, brain trauma, cerebral malaria, sepsis, septic shock, toxic shock syndrome, fever, malgias due to staining, aplastic anemia, hemolytic anemia, idiopathic thrombocytopenia, Goodpasture's syndrome, Guillain-Barre syndrome, Hashimoto's thyroiditis, pemphigus, IgA nephropathy, pollinosis, antiphospholipid antibody syndrome, polymyositis, Wegener's granulomatosis, arteritis nodosa, mixed connective tissue disease, fibromyalgia, asthma, atopic dermatitis, chronic atrophic gastritis, primary biliary cirrhosis, primary sclerosing cholangitis, autoimmune pancreatitis, aortitis syndrome, rapidly progressive glomerulonephritis, megaloblastic anemia, idiopathic thrombocytopenic purpura, primary hypothyroidism, idiopathic Addison's disease, insulin-dependent diabetes mellitus, chronic discoid lupus erythematosus, pemphigoid, herpes gestationis, linear IgA bullous dermatosis, epidermolysis bullosa acquisita, alopecia areata, vitiligo vulgaris, leukoderma acquisitum centrifugum of Sutton, Harada's disease, autoimmune optic neuropathy, idiopathic azoospermia, habitual abortion, hypoglycemia, chronic urticaria, ankylosing spondylitis, psoriatic arthritis, enteropathic arthritis, reactive arthritis, spondyloarthropathy, enthesopathy, irritable bowel syndrome, chronic fatigue syndrome, dermatomyositis, inclusion body myositis, Schmidt's syndrome, Graves' disease, pernicious anemia, lupoid hepatitis, presenile dementia, Alzheimer's disease, demyelinating disorder, amyotrophic lateral sclerosis, hypoparathyroidism, Dressler's syndrome, Eaton-Lambert syndrome, dermatitis herpetiformis, alopecia, progressive systemic sclerosis, CREST syndrome (calcinosis, Raynaud's phenomenon, esophageal dysmotility, sclerodactyly, and telangiectasia), sarcoidosis, rheumatic fever, erythema multiforme, Cushing's syndrome, transfusion reaction, Hansen's disease, Takayasu arteritis, polymyalgia rheumatica, temporal arteritis, giant cell arthritis, eczema, lymphomatoid granulomatosis, Kawasaki disease, endocarditis, endomyocardial fibrosis, endophthalmitis, fetal erythroblastosis, eosinophilic fasciitis, Felty syndrome, Henoch-Schonlein purpura, transplant rejection, mumps, cardiomyopathy, purulent arthritis, familial Mediterranean fever, Muckle-Wells syndrome, and hyper-IgD syndrome.

The compositions encompassing Cas9 of the present invention can also be used as an antiviral agent.

The compositions encompassing Cas9 of the present invention can also be for use in treating arteriosclerosis, including any form thereof.

The compositions encompassing Cas9 of the present invention can also be for use in treating cancer including lung cancer (including small cell lung cancer, non-small cell lung cancer, pulmonary adenocarcinoma, and squamous cell carcinoma of the lung), large intestine cancer, rectal cancer, colon cancer, breast cancer, liver cancer, gastric cancer, pancreatic cancer, renal cancer, prostate cancer, ovarian cancer, thyroid cancer, cholangiocarcinoma, peritoneal cancer, mesothelioma, squamous cell carcinoma, cervical cancer, endometrial cancer, bladder cancer, esophageal cancer, head and neck cancer, nasopharyngeal cancer, salivary gland tumor, thymoma, skin cancer, basal cell tumor, malignant melanoma, anal cancer, penile cancer, testicular cancer, Wilms' tumor, acute myeloid leukemia (including acute myeloleukemia, acute myeloblastic leukemia, acute promyelocytic leukemia, acute myelomonocytic leukemia, and acute monocytic leukemia), chronic myelogenous leukemia, acute lymphoblastic leukemia, chronic lymphatic leukemia, Hodgkin's lymphoma, non-Hodgkin's lymphoma (Burkitt's lymphoma, chronic lymphocytic leukemia, mycosis fungoides, mantle cell lymphoma, follicular lymphoma, diffuse large-cell lymphoma, marginal zone lymphoma, pilocytic leukemia plasmacytoma, peripheral T-cell lymphoma, and adult T cell leukemia/lymphoma), Langerhans cell histiocytosis, multiple myeloma, myelodysplastic syndrome, brain tumor (including glioma, astroglioma, glioblastoma, meningioma, and ependymoma), neuroblastoma, retinoblastoma, osteosarcoma, Kaposi's sarcoma, Ewing's sarcoma, angiosarcoma, and hemangiopericytoma.

The appended Examples indicate that the Cas9 protein of the invention can be used for successfully targeting human breast cancer cells by deleting the oncogene EpCAM. Accordingly, in the treatment of cancer by using the Cas9 protein of the invention the cancer cells may be targeted for gene engineering, e.g. one or more oncogene(s) may be deleted from the cancer cells. Thus, the Cas9 protein of the invention may be used in the treatment of cancer (such as breast cancer), e.g. by targeting the cancer cells for gene engineering.

The present invention also provides a method of treatment wherein the pharmaceutical composition as described above is administered to a subject or patient which suffers from one or more of the diseases mentioned above. Thus, the invention relates to a method of treating a disease, which is based on one or more mutation(s) comprising administering an effective amount of the composition of the invention to a subject in need of such a treatment. Said disease is preferably based on one mutation in the genome. Said disease may be an inheritable disease.

Besides “treatment” the compositions herein can be used for amelioration and/or prevention of any of the above-mentioned diseases. “Treatment” refers, without limitation, to remediation of, improvement of, lessening of the severity of, or reduction in the time course of, a disease, disorder or condition, or any parameter or symptom thereof “Amelioration” refers, without limitation, to any observable beneficial effect. The beneficial effect can be evidenced, for example, by a delayed onset of clinical symptoms of the disease or condition, a reduction in severity of some or all clinical symptoms of the disease or condition, a slower progression of the disease or condition, an improvement in the overall health or well-being of the subject, or by other parameters well known in the art that are specific to the particular disease. Further, what is to be understood by “prevention” is well known in the art. For example, a patient/subject suspected of being prone to suffer from a disorder or disease as defined herein may, in particular, benefit from a prevention of the disorder or disease. The subject/patient may have a susceptibility or predisposition for a disorder or disease, including but not limited to hereditary predisposition. Such a predisposition can be determined by standard assays, using, for example, genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of compositions/medical components before any clinical and/or pathological symptoms are diagnosed or determined or can be diagnosed or determined by the attending physician. “Prevention” includes, without limitation, to avoid the disease or condition from occurring in patient and/or subject that may be predisposed to the disease but does not yet experience or exhibit symptoms of the disease (prophylactic treatment).

The compositions of the invention can also be used for the treatment, prevention and/or amelioration of diseases in combination with conventional therapy for any of the diseases disclosed herein. Such conventional therapies are well known in the art and the skilled person knows any such therapies. “In combination” means that the composition can be administered separately or be formulated as a fixed combination drug. Fixed combination should be understood as meaning a combination whose active principles are combined at fixed doses in the same vehicle (single formula) that delivers them together to the point of application. Fixed combination can mean, e.g., in a single tablet, solution, cream, capsule, gel, ointment, salve, patch, suppository or trans-dermal delivery system.

### Further Definitions

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”. In the present description and claims, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention. It may be advantageous in the practice of the invention to be in compliance with Article 53(c) EPC and Rule 28(b) and (c) EPC.

The term “about” or “approximately” as used herein when referring to a measurable value such as a parameter, an amount, a temporal duration, and the like, is meant to encompass variations of +/−20% or less, preferably +/−10% or less, more preferably +/−1-5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

All references cited herein are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs.

While the invention has been illustrated and described in detail above, such illustration and description are to be considered illustrative or exemplary and not restrictive. It will be understood that changes and modifications may be made by those of ordinary skill. In particular, the present invention covers further embodiments with any combination of features from different embodiments described above and below.

The present invention is additionally described by way of the following illustrative non-limiting examples that provide a better understanding of the present invention and of its many advantages. The following examples are included to demonstrate preferred embodiments of the invention. It should be appreciated by those of skill in the art that the techniques disclosed in the examples which follow represent techniques used in the present invention to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. The claimed benefits of the invention can be shown by the described examples.

Recombinant DNA technology is described, e.g., in Molecular Cloning: A Laboratory Manual, 2nd ed., vol. 1-3, ed. Sambrook et al, Cold Spring Harbor Laboratory Press, Cold Spring Harbor, N.Y., 1989; Current Protocols in Molecular Biology, ed. Ausubel et al., Greene Publishing and Wiley-Interscience, New York, 1992 (with periodic updates) (“Ausubel et al, 1992”), the series Methods in Enzymology (Academic Press, Inc.); Innis et al., PCR Protocols: A Guide to Methods and Applications, Academic Press: San Diego, 1990; PCR 2: A Practical Approach (M. J. MacPherson, B. D. Hames and G. R. Taylor eds. (1995)); Harlow and Lane, eds. (1988) Antibodies, a Laboratory Manual; and Animal Cell Culture, R. I. Freshney, ed. (1987). General principles of microbiology are described, e.g., in Davis, B. D. et al, Microbiology, 3rd edition, Harper & Row, publishers, Philadelphia, Pa. (1980).

### EXAMPLES

### Example 1

Materials and Methods Used for Examples 2-6

1. DNA Handling

Plasmid DNA preparation (QIAprep Spin MiniPrep Kit, Qiagen), polymerase chain reaction (PCR) (Phusion High Fidelity Polymerase, Thermo Scientific; Taq DNA polymerase, Fermentas), DNA digestion with restriction enzymes (Thermo Scientific), DNA ligation (T4 DNA Ligase, Fermentas), purification of PCR products (QIAquick PCR Purification Kit, Qiagen), agarose gel electrophoresis and polyacrylamide gel electrophoresis were performed according to the manufacturer's instructions and using standard protocols. Site-directed mutagenesis was performed according to Kirsch 1998 26 Nucleic Acids Res. 1848.

2. RNA In Vitro Transcription

RNAs used in the study were in vitro transcribed with the AmpliScribe-T7 Flash Transcription kit (Epicentre) according to manufacturer's instructions. The templates for the reaction were either oligonucleotides or were generated by PCR. The transcription products were sodium acetate/ethanol-precipitated and purified over 10% polyacrylamide urea gel. The corresponding bands were excised from the gel and RNA was extracted with EluRNA solution (0.3 M sodium acetate, 0.5 mM EDTA, 0.1% SDS) at 50° C. and precipitated in 100% ethanol at −20° C. for 2 hours or overnight. This procedure was repeated twice. The pellets were washed in 70% ethanol and air-dried. After drying, pellets were resuspended in RNase-free water (Epicentre). RNA concentration was determined by measuring absorbance at 260 nm with NanoDrop. Equimolar amounts of tracrRNA and crRNA were annealed in 5× RNA annealing buffer (1 M NaCl, 100 mM HEPES, pH 7.5) on 95° C. for 5 minutes, and then slowly cooled to room temperature. Dual-RNAs were stored at −20° C.

3. Cas9 Protein Purification

Escherichia coli NiCo21 (DE3) competent cells (New England Biolabs) were transformed with overexpression plasmids encoding wild-type or mutant S. pyogenes Cas9. Bacterial cells were grown in LB media on 37° C. until an OD₆₀₀ 0.6-0.8, after which the protein expression was induced with 0.5 mM IPTG. Cells were grown overnight at 13° C. Afterwards, they were harvested by centrifugation and the pellets were washed with STE buffer (100 mM NaCl, 10 mM Tris-HCl pH 8, 1 mM EDTA, pH 8). Pellets were resuspended in lysis buffer (20 mM HEPES pH 7.5, 500 mM KCl, 0.1% Triton X-100, 25 mM imidazole), the cells were disrupted by sonification and harvested by centrifugation (16000 rpm, SS-34 rotor, Thermo Scientific). The lysates were applied to Ni-NTA Agarose (Qiagen) or Talon (Sigma-Aldrich) affinity chromatography matrix and incubated for 1 h at 4° C. The affinity matrix was washed with lysis buffer and wash buffer (20 mM HEPES pH 7.5, 300 mM KCl, 25 mM imidazole), after which the proteins were eluted with elution buffer (20 mM HEPES pH 7.5, 150 mM KCl, 0.1 mM DTT, 250 mM imidazole, 1 mM EDTA). The elution fractions were analyzed by sodium dodecyl sulphate-polyacrylamide gel electrophoresis (SDS-PAGE). Protein-containing fractions were further purified over chitin beads (New England Biolabs). Chitin beads were equilibrated with Buffer A (20 mM HEPES pH 7.5, 100 mM KCl), after which the protein fractions were added and incubated for 1 h at 4° C. The beads were added to a column, Cas9 protein was eluted and the fractions were again analyzed by SDS-PAGE. Protein-containing fractions were dialyzed against dialysis buffer (20 mM HEPES pH 7.5, 150 mM KCl, 50% glycerol) overnight. Protein concentration was determined with Bradford assay and purity was assessed by measuring A₂₆₀/A₂₈₀ ratio.

4. Preparation of Substrates for Electrophoretic Mobility Shift Assays

4.1 Substrates Amplified from Plasmids

DNA substrates were synthesized by PCR of plasmids with wild-type (wt) and mutated protospacer 2 (pEC576-pEC608) using primers OLEC4816 and OLEC4817. Products were precipitated with sodium acetate and ethanol and purified over 1.5% agarose gel in TBE buffer. The corresponding bands were excised from the gel and purified using QIAquick Gel Extraction Kit (Qiagen) following manufacturer's instructions. DNA concentration was determined by measuring absorbance at 260 nm using NanoDrop, after which molarity was calculated.

4.2 Oligonucleotide Substrates

Substrates containing the PAM, DNA target sequence (wt or with desired mutations) and flanking regions (116-nt long) were ordered as HPLC-purified oligonucleotides (Sigma). To generate a double-stranded EMSA (electrophoretic mobility shift assay) substrate, oligonucleotides containing the target and non-target DNA strand were annealed in 5×RNA annealing buffer at 95° C. for 5 minutes and then left at room temperature for slow cooling. The substrates were purified over 6% polyacrylamide gel in TBE buffer, and the corresponding bands were excised from the gel. Gel pieces containing the samples were incubated overnight on 4° C. in 1×TE buffer (1 M Tris-HCl pH 8, 0.5 M EDTA), after which DNA was precipitated with sodium acetate and ethanol. DNA pellets were dissolved in Milli-Q water.

5. Electrophoretic Mobility Shift Assay

Substrates for EMSAs were radiolabeled with [γ-³²P]-ATP (Hartmann Analytics) using T4 polynucleotide kinase (Fermentas) and purified on Illustra Microspin G-25 columns (GE Healthcare). Binding reactions with Cas9 protein and 2-molar excess of dual-RNA were preincubated in Binding buffer (20 nM Tris-HCl pH 7.5, 100 mM KCl, 5 mM CaCl₂*2H₂O, 5% glycerol, 1 mM DTT) for 15 minutes at 37° C., prior to the addition of 1 nM labeled DNA substrates. Binding reactions took place on 37° C. for 1 hour. Protein-DNA complexes were separated from unbound DNA by 5% native polyacrylamide gel electrophoresis in 0.5×TBE buffer with 5 mM CaCl₂*2H₂O. The gels were exposed to autoradiography film overnight, which were then visualized by phosphorimaging. Results of at least three independent experiments were quantified with Gel Analyzer and analyzed by non-linear regression analysis using Origin Software.

6. Kinetic Cleavage Assay

Dual RNA (20 nM) and Cas9 (10 nM) were preincubated for 15 minutes at 37° C. in KGB buffer (100 mM potassium glutamate, 25 mM Tris-acetate pH 7.5, 10 mM Mg-acetate, 0.5 mM 2-mercaptoethanol, 10 mg/ml bovine serum albumin) McClelland 1988 16 Nucleic Acids Res. 364. Directly after preincubation, plasmid DNA (5 nM) containing wt or mutated protospacer was added to the reactions and incubated for 90 minutes at 37° C. At several time points, samples were withdrawn and the reaction was stopped by addition of 5× loading buffer (250 mM EDTA, 30% glycerol, 1.2% SDS, 0.1% bromophenol blue). Cleavage products were resolved on a 1% agarose gel electrophoresis in 1×TAE buffer. DNA was visualized by ethidium bromide staining. Band intensity of open circular, linear and supercoiled DNA was analyzed by densitometry to determine the kinetics of the cleavage reaction. Data obtained from at least three independent experiments were fitted by non-linear regression analysis using Origin Software.

7. In Vivo Activity of Cas9 in HaCat and MCF7 Cell Lines

Cells were seeded in 24-well plates 24 hours prior to transfection (100000 cells/nil). The transfection was done according to the following protocol:

1. Dilute 0.5 μg (500 ng) DNA into 50 μl jetPRIME® buffer (supplied). Mix by vortexing.

2. Add 1 μl jetPRIME™, vortex for 10 s, spin down briefly.

3. Incubate for 10 min at RT.

4. Add 50 μl transfection mix per well drop wise onto the cells kept in regular cell growth medium, and distribute evenly.

5. Gently rock the plates back and forth and from side to side.

6. Replace transfection medium after 4 h by 0.5 ml of growth medium and return the plates to the incubator.

MCF7 cells were transfected with 500 ng of plasmid DNA, whereas HaCat cells were transfected with 250 ng of plasmid DNA.

Transfected cells were selected by adding puromycin one day after transfection (2 μg/ml for MCF7 cells, 1 μg/ml for HaCat cells). Growth medium with puromycin was replaced by standard growth medium (advanced DMEM with 10% FBS, 2 mM L-glutamine and penicillin-streptomycin) after 2 days. MCF7 cells were analyzed by FACS 10 days post transfection, HaCat cells 13 days post transfection.

8. Bacterial Survival Assay

The bacterial survival assay to measure Cas9 cleavage in vivo is based on a three-plasmid system. The three plasmids encode RFP, Cas9 and sgRNA, respectively. Cas9 is expressed under the control of the arabinose promoter, the sgRNA targeting the 5′ region of rfp is constitutively expressed and RFP expression is controlled by the T7 promoter and the lacO operator. The bacterial cells used in the assay are E. coli SE4 (Delphi genetics), an engineered derivative of BL21DE3, which in addition encodes the toxin CcdB. The corresponding antitoxin CcdA is encoded on the RFP expressing plasmid. E. coli SE4 was transformed with these three plasmids in a consecutive manner (1^{st}: RFP containing plasmid, 2^{nd}: plasmid encoding wt sgRNA or sgRNA 3^{rd}: plasmid encoding Cas9_wt or mutant Cas9 proteins). Cells were inoculated either LB medium with Sm, Cb, Cm and 1% glucose (suppressing conditions), or LB medium with Sm, Cb, Cm with 33 mM arabinose and 0.1 mM IPTG (inducing conditions). Under suppressing conditions (1% glucose), neither RFP nor Cas9 is expressed and CcdB is neutralized by the presence of CcdA. Under inducing conditions (0.1 mM IPTG and 33 mM arabinose) three possible scenarios can take place. 1) Cas9 is cleavage and binding deficient. In this case, RFP and CcdA are expressed, the cells grow and fluorescence can then be detected. 2) Cas9 is cleavage deficient, but able to bind its target site. The cells grow since CcdA is still expressed, but rfp expression is repressed by Cas9 binding to the 5′ region of the gene. 3) Cas9 is able to bind and cleave the RFP and CcdA encoding plasmid. This leads to death of the cells, since the CcdA antitoxin is no longer expressed and the toxin CcdB can no longer be neutralized. To distinguish between these scenarios, the OD600 nm and red fluorescence units (RFUs) (excitation wavelength 555 nm, emission wavelength 588 nm) were measured with a fluorescence plate reader (Biotek) in 5 minute intervals during a 10 hour kinetic experiment, at 37° C. with shaking. After subtraction of the blank samples, survival was calculated by dividing the OD600 nm at inducing conditions by the OD600 nm at suppressing conditions. Statistical analysis of at least five replicates was performed using Origin Software (OriginLab, Northampton, Mass.).

### Example 2

The Influence of Mismatches Between crRNA and DNA on Cleavage and Binding of Streptococcus pyogenes Cas9

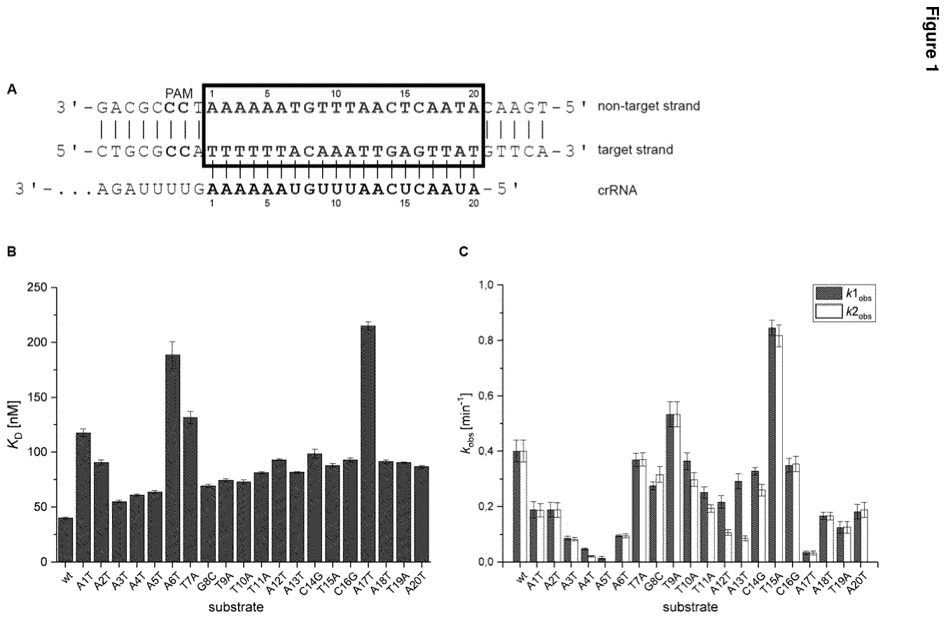

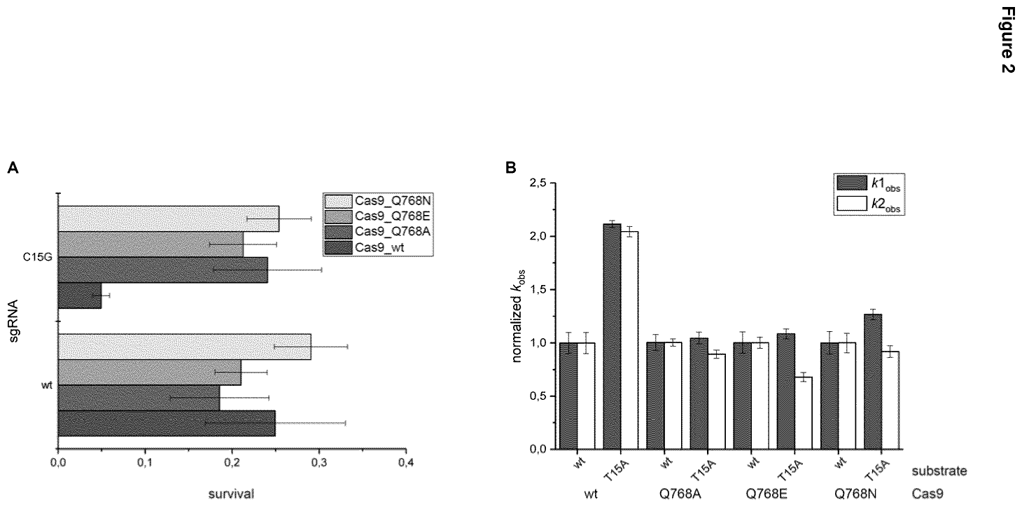

To investigate the influence of mismatches between the crRNA and DNA on Cas9 cleavage and binding, and to characterize seed sequence requirements in greater detail, we performed kinetic cleavage assays and EMSA with Cas9_wt on the wt substrate and substrates containing single mismatches to the crRNA (FIG. 1). These mismatches prevented base-pairing of the crRNA to the target at the specific position. The numbering of mismatch positions is from 1 to 20, where 1 is the most PAM-proximal base of the target and 20 the most PAM-distal base of the target.

Results show that Cas9 cleavage rates are markedly decreased on substrates with mismatches at positions 3, 4 and 5, compared to the wt substrate that is complementary to the crRNA. The binding affinity of Cas9 for substrates A3T-A5T is comparable to that of the wt substrate. This implies that the observed effect is due to impairment in protein catalysis, which is also in agreement with the fact that Cas9 cleaves the target upstream of the PAM, between the 3^{rd} and 4^{th} base (Jinek 2012 337 Science 816.). Possible explanations for this result is that the conformational change which brings the HNH domain closer to the cleavage site is not able to occur, or that the HNH domain is trapped in a catalytically inactive state (Dagdas 2017 3 Sci. Adv. eaao0027; Sternberg 2015 527 Nature 110). Furthermore, the scissile phosphate might not be accessible for cleavage. Mismatches at positions 6 and 17 highly impair DNA binding which is reflected in the reduced cleavage rates. Cas9 has two active sites that each cleave one strand of the DNA. Therefore, two separate cleavage events and rates can be observed. Interestingly, cleavage rate k1_{obs} (which represents the disappearance of the supercoiled form of the plasmid) is higher than the cleavage rate k2_{obs} (which represents the appearance of the linear form of the plasmid) on substrates T10A-C14G. This suggests that one Cas9 endonuclease domain has a faster cleavage rate than the other, resulting in the accumulation of the nicked intermediate (open-circular form of the plasmid). Cleavage assays on linear substrates containing mismatches at the same positions indicated that the cleavage by RuvC domain is slower (results not shown). Substrates containing mismatches at the PAM-distal part of the protospacer (namely from position 17 until position 20) were bound weaker than the wt substrate; the observed cleavage rates on these substrates were reduced accordingly. This result is in agreement with reports showing that complementarity at the PAM-distal end of the target is important for cleavage (Cencic 2014 9 PLoS ONE e109213) and that mismatches at these positions prevent conformational activation of the HNH domain and hence inhibit cleavage (Dagdas 2017 3 Sci. Adv. eaao0027; Sternberg 2015 527 Nature 110).

### Example 3

Arginine 63 and 66 from the Bridge Helix Influence Cas9 Cleavage and Binding.

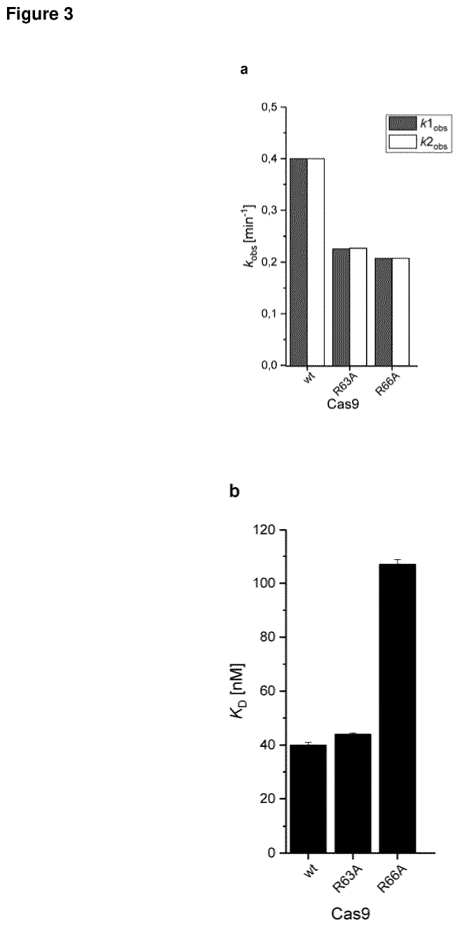

The bridge helix of S. pyogenes Cas9 is one of two linkers connecting the lobes of Cas9, and contains a cluster of arginine residues (Nishimasu 2014 156 Cell 935). There is a high degree of conservation of these residues throughout the type II CRISPR-Cas system (Chylinski 2014 42 Nucleic Acids Res. 6091). A study of Francisella novicida Cas9 demonstrated that R59A mutant (equivalent to R70A in S. pyogenes Cas9) is not able to bind tracrRNA and a small CRISPR-Cas-associated RNA (scaRNA) (Sampson 2013 497 Nature 254). Crystal structure of S. pyogenes Cas9 bound to sgRNA and target DNA showed that arginine residues from the bridge helix (namely R63, R66, R69, R70, R71, R74, R75 and R78) interact with the sgRNA via single or multiple salt bridges with the phosphate backbone along the seed region (Nishimasu 2014 156 Cell 935). We focused on R63 and R66 and investigated how these two residues influences target binding and cleavage. The cleavage and binding properties of Cas9_R63A and Cas9_R66A were tested on the substrate with a target site fully complementary to the crRNA using kinetic cleavage assays and EMSAs (FIG. 3).

The results revealed that Cas9_R63A has binding constants comparable to Cas9_wt, but its cleavage rates are slower than that of the Cas9_wt. This implies that R63 is important for catalysis. Cas9_R66A has a higher binding constants compared to Cas9_wt, meaning that it does not bind DNA efficiently. Consequently, the cleavage rate of R66 is also slower when compared to Cas9_wt. The results are in agreement with the fact that R66 makes multiple contacts with the sgRNA phosphate backbone (Nishimasu 2014 156 Cell 935).

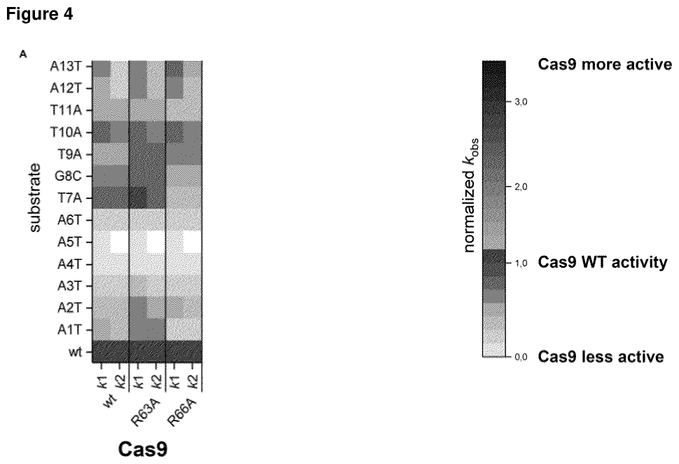

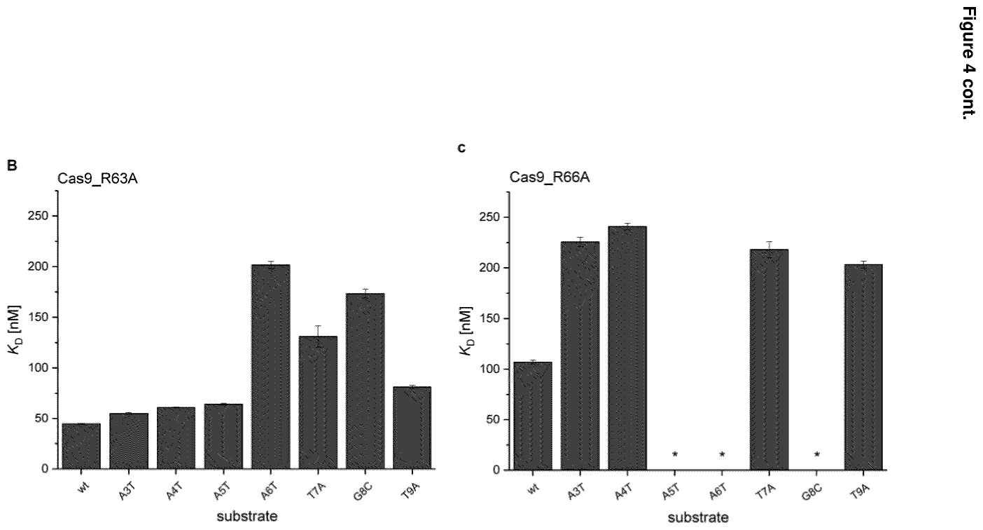

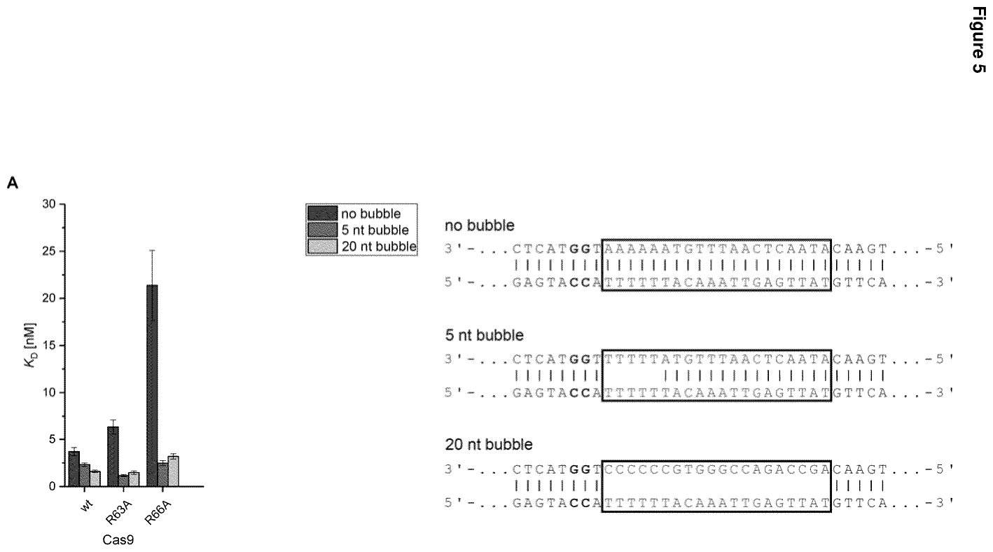

Next, we wanted to investigate if R63 and R66 influence the sensitivity of Cas9 to mismatches between the crRNA and DNA. Thus, we tested Cas9_R63A and Cas9_R66A for cleavage and binding of substrates containing mismatches in the target site. Cleavage rates of Cas9_R63A and Cas9_R66A on mismatched substrates are similar to or slower than on the wt substrate. The reason for this is an impaired binding ability for several substrates containing a mismatch in the PAM-proximal region of the DNA. This suggests that removal of these residues increases sensitivity of the protein to the mismatches, meaning that the specificity is enhanced (FIG. 4).

According to the kinetic model for the specificity of RNA-guided nucleases, when the protein affinity for both the on-target and off-target sequences decreases, the specificity of the nuclease for the target increases (Bisaria 2017 4 Cell Syst. 21). Therefore, a Cas9 variant with an increased dissociation constant (K_{D}) and a decreased cleavage rate (k_{obs}) should have enhanced specificity. Cas9_R66A has binding defects and slower cleavage rate on both wt and mismatched substrates, whereas Cas9_R63A has a binding defect on the substrate with mismatched position 8 and slower cleavage rate on the mismatched substrates.

### Example 4

Glutamine 768 is Involved in Cas9 Sensitivity to PAM-Distal Mismatches

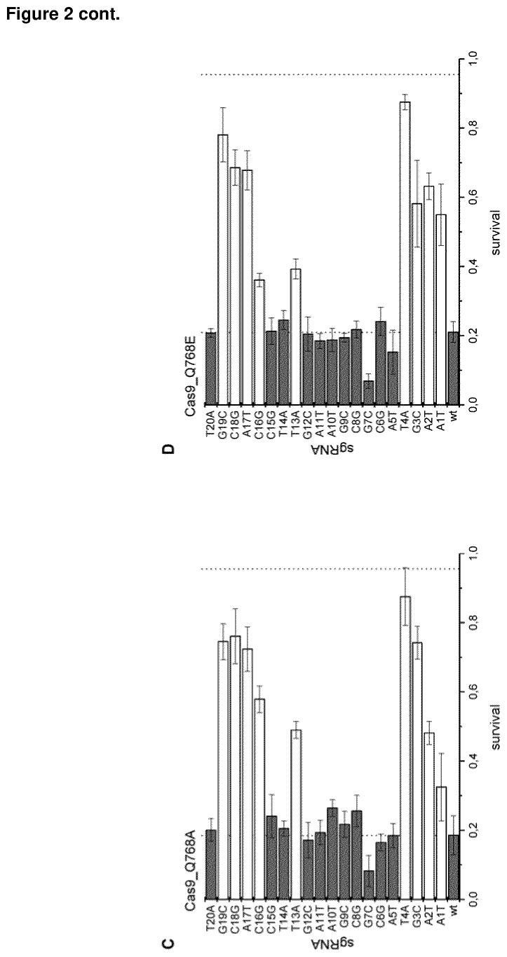

To identify Cas9 residues that could mediate the enhanced cleavage rate in the presence of a mismatch at position 15 (a mismatch which is representative for a PAM-distal mismatch), we examined the crystal structure of S. pyogenes Cas9 complexed with sgRNA and target DNA. The side-chain of glutamine 768 (Q768), located at the border between the RuvC and HNH domains of Cas9, is in proximity to the target DNA at position 15. We hypothesized that a mismatch might perturb the contact between Q768 and the RNA:DNA hybrid in this region, and as a result affect the cleavage rate. To find out whether Q768 is responsible for this, we replaced this residue with alanine, glutamate or asparagine and tested the resulting mutants in bacteria (FIG. 2). Expression of Cas9_Q768A, Cas9_Q768E and Cas9_Q768N resulted in survival comparable to bacteria where Cas9_wt was expressed in the case of full complementarity between the sgRNA and target DNA. However, in the presence of the sgRNA C15G (i.e. an exemplary PAM-distal mismatch at position 15), Cas9_Q768A, Cas9_Q768E and Cas9_Q768N showed increased survival compared to Cas9_wt, indicating that removal of Q768 increases the specificity of Cas9 if there is a mismatch on position 15. All three mutants were also tested in vitro (FIG. 2). Cleavage rates of Cas9_Q768A and Cas9_Q768E on the wt and T15A (i.e. an exemplary PAM-distal mismatch at position 15) substrates were either in the same range, or slightly slower on T15A, while for Cas9_Q768N the first cleavage rate was faster on T15A substrate compared to the wt substrate, although not to the same extent as seen for Cas9_wt.

We reasoned that since Q768 affects Cas9 sensitivity to a mismatch on position 15, its removal might also influence the sensitivity of Cas9 to other PAM-distal mismatches. Hence, we tested the cleavage of the rfp target on the reporter plasmid by Cas9_Q768A and Cas9_Q768E in E. coli with mismatched sgRNAs (FIG. 2). Bacteria where both mutants were individually expressed showed increased survival in the presence of sgRNAs with distal mismatches at positions 13 and 15-19 when compared to the wt sgRNA and also when compared to survival obtained with Cas9_wt with the same sgRNAs. This indicates that the mutants cleaved the target to a lesser extent than Cas9_wt in the presence of PAM-distal mismatches.

### Example 5

Combination of R63A or R66A with Q768A in Cas9 Enhances Sensitivity to Mismatches

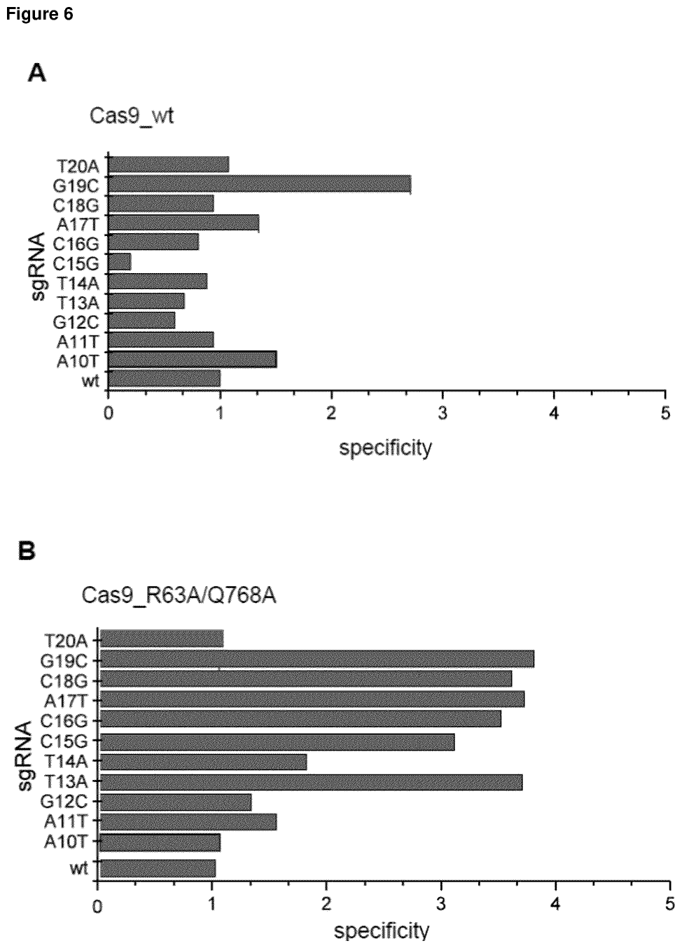

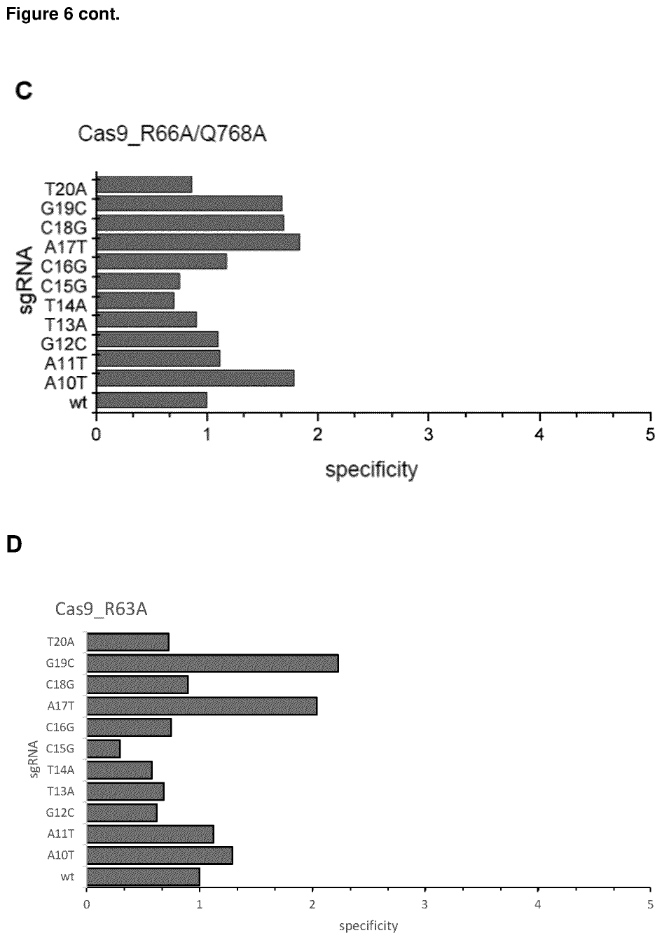

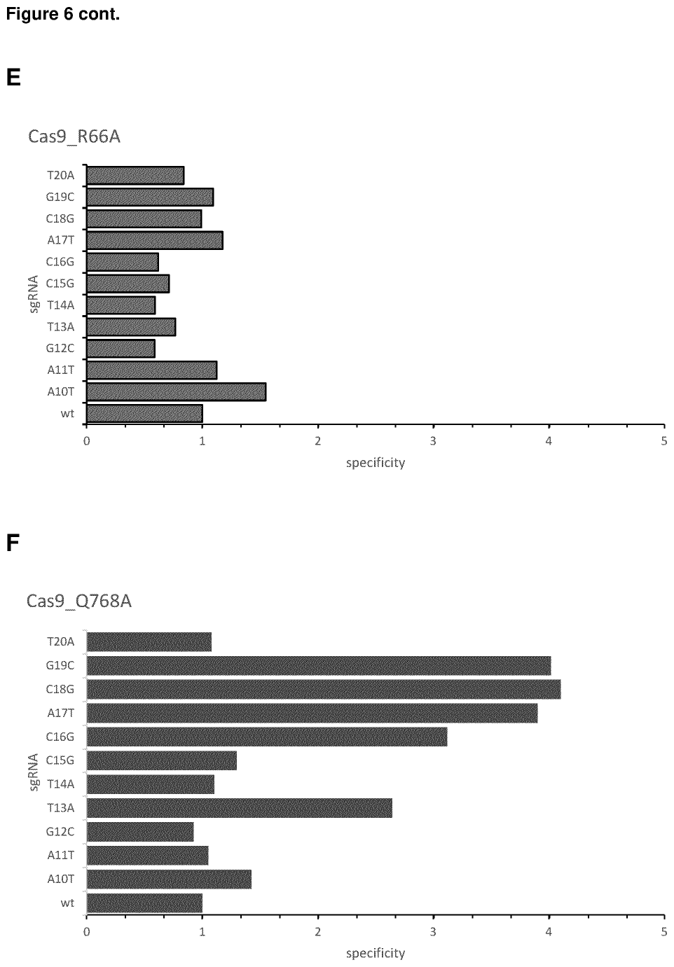

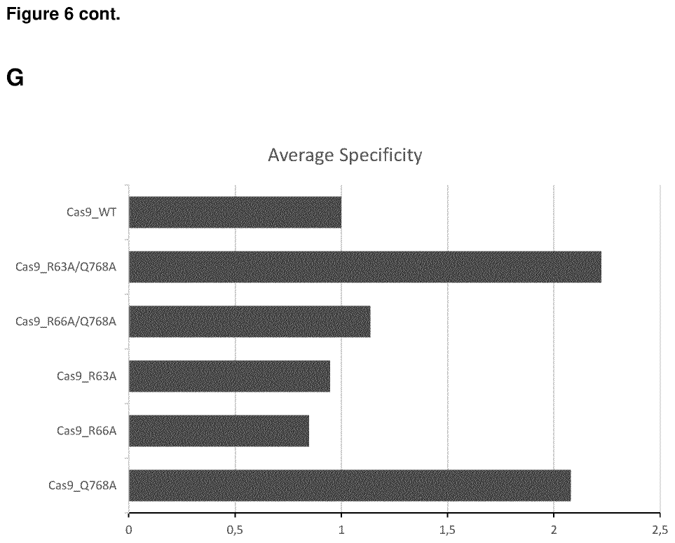

We describe above that mutations R63A and R66A increase Cas9 sensitivity to mismatches in the PAM-adjacent part of the target DNA, and the mutation Q768A increases sensitivity to PAM-distal mismatches. We asked whether double mutations of these residues would have a superior effect on specificity compared to wt Cas9. We determined the in vitro cleavage rates of Cas9_R63A/Q768A and Cas9_R66A/Q768A and tested them for cleavage in the presence of mismatched sgRNAs in vivo (FIG. 6). Overall, the cleavage rates of Cas9_R63A/Q768A and Cas9_R66A/Q768A on the mismatched substrates were lower than on the wt substrate, showing that these mutants have enhanced specificity compared to wt Cas9. We observed a similar trend of mismatch sensitivity for both mutants in the in vivo assay. To directly compare the mutants with Cas9_wt, we determined their specificity by dividing the survival in the presence of mismatched sgRNAs by survival in the presence of wt sgRNA (FIG. 6). Compared to Cas9_wt (FIG. 6a), Cas9_R63A/Q768A showed enhanced specificity in the presence of mismatches (FIG. 6b). The effect is also shown for Cas9_R66A/Q768A (FIG. 6c). The slightly lower increase in specificity of Cas9_R66A/Q768A can be attributed to lower on-target activity.

Taken together, these results show that Cas9_R63A/Q768A and Cas9_R66A/Q768A are sensitive to mismatches and enhance specificity compared to wt Cas9.

The specificity of the single mutants Cas9_R63A (FIG. 6D), Cas9_R66A (FIG. 6E) and Cas9_Q768A (FIG. 6F) was also determined. All of the single mutants displayed significantly lower specificity compared to the double mutant Cas9_R63A/Q768A. In FIG. 6G a comparison of the Cas9_wt, single and double mutants is shown. Values for the specificities of the mutants are as follows (normalized towards Cas9_wt):

TABLE A

| | Value for the | Value for the |
|---|---|---|
| | specificities | specificities in % |
| | (normalized to | (normalized to |
| Species | Cas9_wt) | Cas9_wt) |
| Cas9_wt | 1 | 100 |
| Cas9_R63A/Q768A | 2.224 | 222.4 |
| Cas9_R66A/Q768A | 1.136 | 113.6 |
| Cas9_R63A | 0.947 | 94.7 |
| Cas9_R66A | 0.848 | 84.8 |
| Cas9_Q768A | 2.081 | 208.1 |

As can be seen from FIG. 6G and the values indicated above, the double mutant Cas9_R63A/Q768A displays the highest increase in specificity. The double mutant Cas9_R63A/Q768A is 2.224 times more specific than Cas9_wt. Thus, the double mutant Cas9_R63A/Q768A displays an 222.4% increase in specificity compared to Cas9_wt.

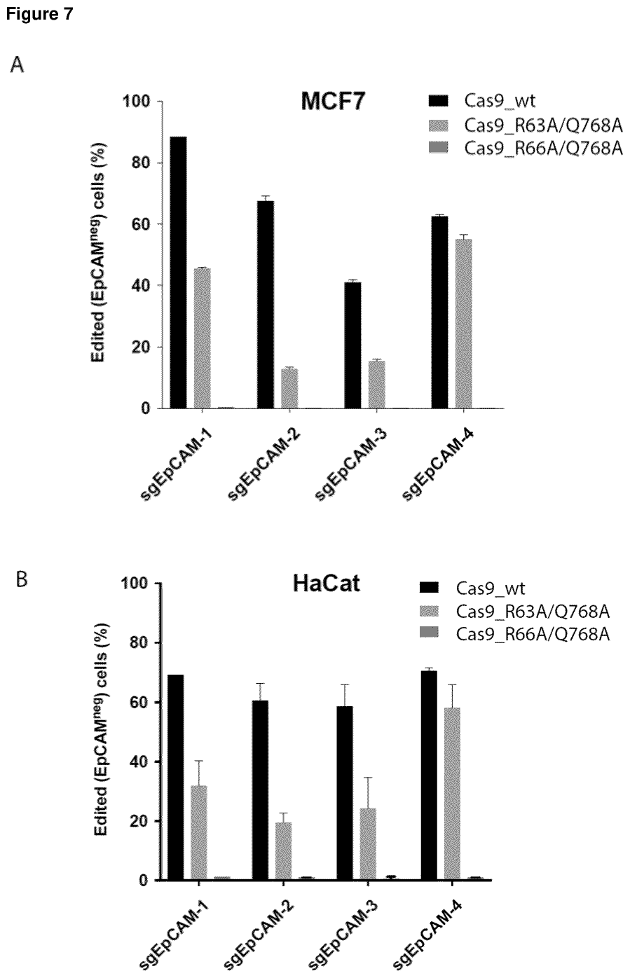

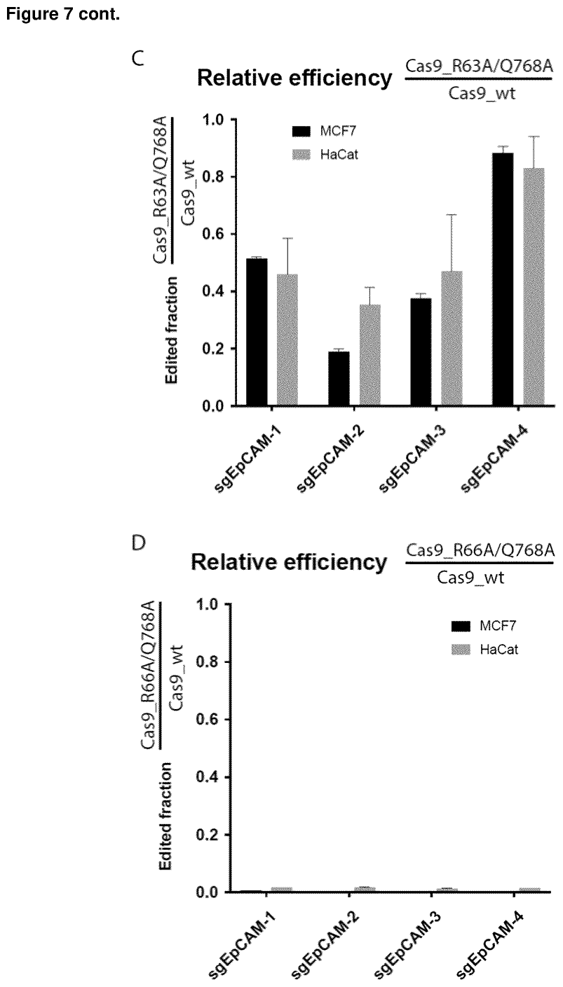

A comparison of the phenotypes of the single mutants with that of the double mutants revealed that the sum of the specificities of the single mutants does not equal the simple combination of both single mutant phenotypes (see FIG. 6G and the above Table A). Based on the phenotype of the single mutants (FIG. 6D-F), both double mutant Cas9_R66A/Q768A and Cas9_R63A/Q768A should display equal specificity as both Cas9_R66A and Cas9_R63A display similar specificity (FIG. 6A and the above Table A). However, Cas9_R63A/Q768A clearly outperforms Cas9_R66A/Q768A (see FIG. 6A and the above Table A). In addition, Cas9_R66A/Q768A is also not active in human cells (FIGS. 7A and 7B), whereas the double mutant Cas9_R63A/Q768A is active in human cells (FIGS. 7A and 7B).

Further, the increase in specificity of the double mutant Cas9_R63A/Q768A is not merely the sum of the specificities of the single mutants Cas9_R63A and Cas9_Q768A. The double mutant Cas9_R63A/Q768A shows a synergistic effect. This can be seen from FIG. 6G and the above Table A. When the specificity of the single mutant Cas9_R63A is added to the specificity of the single mutant Cas9_Q768A, then one would not merely obtain the sum of the specificities of the single mutants. In contrast, there is an increased specificity (i.e. a synergistic effect) observed in the double mutant Cas9_R63A/Q768A, which is higher than the sum of the specificity of the two single mutants Cas9_R63A and Cas9_Q768A.

Furthermore, the double mutant Cas9_R63A/Q768A outperforms other mutants not only in total increased specificity, but also when considering specific positions.

For instance, at position 15, Cas9_R63A (FIG. 6D) has similar specificity as Cas9_wt (FIG. 6A) and Cas9_Q768A has only slightly increased specificity compared to Cas9_wt (FIG. 6F). In contrast, at position 15, the double mutant Cas9_R63A/Q768A (FIG. 6B) has highly increased specificity.

Also, at position 15, the single mutant Cas9_R66A (FIG. 6E) outperforms Cas9_R63A (FIG. 6D) with respect to specificity. However, the double mutant Cas9_R66A/Q768A (FIG. 6C) has less specificity at position 15 than both single mutants. This is in contrast to the double mutant Cas9_R63A/Q768A (FIG. 6B), where the observed specificity at position 15 is highly increased when compared to both single mutants.

Furthermore, for instance, at positions 13, 16 and 18, both single mutants Cas9_R63A (FIG. 6D) and Cas9_R66A (FIG. 6E) show similar specificities. However, at said positions 13, 16 and 18, the double mutant Cas9_R63A/Q768A (FIG. 6B) shows highly increased specificity compared to the double mutant Cas9_R66A/Q768A (FIG. 6C).

Moreover, at position 19, both single mutants Cas9_R63A (FIG. 6D) and Cas9_R66A (FIG. 6E) are slightly less specific than Cas9_wt (FIG. 6A). In contrast, the double mutant Cas9_R63A/Q768A (FIG. 6B) shows highly increased specificity at position 19, whereas the double mutant Cas9_R66A/Q768A (FIG. 6C) is less specific at position 19.

In sum, these results show that the double mutant Cas9_R63A/Q768A has increased specificity compared to the Cas9_wt and compared each of the single mutants Cas9_R63A and Cas9_Q768A. A synergistic effect regarding specificity is observed in the double mutant Cas9_R63A/Q768A.

### Example 6

Arginine 63 Stabilize the R-Loop in the Presence of Mismatches

Finally, we wanted to investigate the underlying mechanism of how R63 influence Cas9 specificity. Our previous experiments indicate that these residues might facilitate R-loop formation (data not shown). We hypothesise that R63 may positively affect the R-loop stability in the presence of a mismatch, thus making the protein more tolerant to mismatches and therefore less specific. To investigate how R63 directly influence specificity, we performed binding assays on two sets of substrates in which mismatches between the target and non-target strands opens the DNA and facilitates strand separation and R-loop formation.

The first set of substrates allowed full base-pairing between the crRNA and target DNA strand, but included mismatches between the target and non-target DNA strand in order to create a bubble at the positions where R63 contact the RNA:DNA hybrid. The second set of substrates contained mismatches between the crRNA and target DNA at a specific positions, and two further mismatches between the target and non-target DNA strands to facilitate the R-loop formation. We tested Cas9_R63A and as a control, we tested binding of Cas9_wt on the same substrates. The effect of these residues on the R-loop stability is described below.

Cas9_R63A binds the wt substrate comparable to Cas9_wt, but has a binding defect on the substrate with a mismatch at position 8 (substrate G8C) (FIG. 4). This suggests that R63 stabilizes the R-loop in the presence of a mismatch on position 8. Therefore, when this residue is replaced by an alanine, binding is impaired due to loss of the stabilizing effect. However, if the DNA substrate containing the G8C mismatch is opened at the next two positions (namely 9 and 10), Cas9_R63A can bind this substrate with the same affinity as the wt substrate. This shows that the negative effect of removing R63 is neutralized by opening the substrate and facilitating R-loop formation. These results show that R63 stabilizes the R-loop in the presence of a mismatch at position 8, and thereby lowers the sensitivity of Cas9 to this mismatch.

### Example 7

Materials and Methods Used for Example 7 and 8

1. DNA Handling

Plasmid DNA preparation (QIAprep Spin MiniPrep Kit, Qiagen), polymerase chain reaction (PCR) (Phusion High Fidelity Polymerase, Thermo Scientific; Taq DNA polymerase, Fermentas), DNA digestion with restriction enzymes (Thermo Scientific), DNA ligation (T4 DNA Ligase, Fermentas), purification of PCR products (QIAquick PCR Purification Kit, Qiagen), agarose gel electrophoresis and polyacrylamide gel electrophoresis were performed according to the manufacturer's instructions and using standard protocols. Site-directed mutagenesis was performed according to Kirsch 1998 26 Nucleic Acids Res. 1848.

2. Human Cell Culture and Transfections MCF-7 cells were cultured at 37° C. with 5% CO₂ in advanced DMEM (Thermo Scientific) supplemented with 10% heat-inactivated fetal bovine serum (FBS) (Thermo Scientific), 2 mM GlutaMax (Thermo Scientific) and penicillin-streptomycin (Sigma-Aldrich). HEK293 cells (Table 3) were cultured at 37° C. with 5% CO₂ in DMEM (Sigma-Aldrich) supplemented with 10% FBS (Gibco), 2 mM L-Glutamine (Sigma-Aldrich) and Normocin (Invivogen).

Cells were seeded in 6-well or 24-well plates 24 hours prior to transfection at a density of 100.000 cells per ml. Transfections of plasmids with sgRNAs targeting EpCAM were performed with the jetPRIME™ transfection reagent (Polyplus) according to the manufacturer's instructions. MCF-7 cells were transfected with 500 ng of plasmid DNA. Transfected cells were selected by adding 2 μg/ml of puromycin (Sigma-Aldrich) one day after transfection. Growth medium with puromycin was replaced by standard growth medium after 2 days. Cells were analyzed by flow cytometry 8-10 days post transfection. HEK293 cells were transfected with plasmids p1490-1492, p1498-1500 in 24-well plates using Lipofectamine 3000 (Invitrogen) and 1 μg of plasmid according to manufacturer's protocol. Transfected cells were selected with 1 μg/ml puromycin (Invivogen) for 3 days starting at day 1 post transfection. Cells were collected 5 days post transfection and lysed with the DirectPCR Lysis Reagent (Cell) (Viagen), supplemented with Proteinase K (ThermoFisher) according to the manufacturer's protocol. Genomic DNA extracted from these cells was used for PCR amplification of on- and off-target sites and amplicon sequencing (see below).

3. Flow Cytometry

To determine the levels of EpCAM editing by Cas9_wt, Cas9_R63A/Q768A and Cas9_R66A/Q768A, cells were stained with human EpCAM (CD326) antibody conjugated to FITC (Miltenyi Biotec) according to the manufacturer's instructions. Dead cells were excluded from the analysis by staining with the 7-AAD viability staining solution (BioLegend). Samples were acquired with the Sony SH800 cell sorter and data were analyzed with the FlowJo software version 10.5.3 (Tree Star).

4. Construction of Plasmids for In Vivo Gene Editing in Eukaryotic Cells

Oligonucleotides containing sgRNA spacers (OLEC10121-10132, OLEC10341-10479) targeting the EpCAM gene (fully complementary or with single point mutations that caused a mismatch to the target DNA sequence) were phosphorylated with T4 polynucleotide kinase (Fermentas) and annealed to generate the double-stranded inserts. To obtain the variants Cas9_R63A/Q768A and Cas9_R66A/Q768A, site-directed mutagenesis was performed on the plasmid pCROPseq Cas9_wt, which is based on CROPseq-Guide-Puro (Addgene #86708) (Datlinger, Nat Methods. 2017 March; 14(3):297-301 (PMID: 28099430)) with an added human codon optimized SpCas9 containing a C-terminal NLS tag. Cas9-encoding plasmids were digested with Esp3I (Thermo Scientific) and dephosphorylated with alkaline phosphatase (Thermo Scientific).

For cloning of sgRNAs containing spacers targeting the VEGFA sites 1 and 3, and the EMX/target site 4, Cas9-encoding plasmids were digested with BpiI FD (ThermoFisher) and purified (GeneJET Gel Extraction Kit, ThermoFisher) following the manufacturer's instructions. Oligonucleotides containing sgRNAs (CR3373-3378) were mixed and annealed by denaturation and subsequent slow cooling. The inserts were cloned into the digested vectors using T4 DNA ligase (ThermoFisher) to generate full sgRNAs expressed under the control of the U6 promoter.

5. Amplicon Sequencing

On-target and off-target sites were amplified by PCR with Phusion High Fidelity DNA Polymerase (Thermo Scientific) using primers listed below. The following PCR program was used: (98° C., 10 s; appropriate annealing temperature for each primer pair, 15 s; 72° C., 30 s)×35 cycles (30 cycles for nested PCRs), with the addition of DMSO if necessary. The libraries were prepared with 10 ng DNA for each sample using the KAPA HyperPrep-Kit (Roche), according to the manufacturer's instructions and without fragmentation and size selection. This was followed by 8 cycles of PCR to add sequencing adapters. After quality control, libraries with similar size were pooled together, resulting in four pools of 14 libraries, respectively one pool of 20 libraries. These pools were quantified with the KAPA Library Quantification Kit (Roche), normalized to 2 nM and pooled again equimolarly to load them on the MiSeq.

Fastq data was analyzed using the ampliCan (Labun, Genome Res. 2019 May; 29(5):843-847 (PMID:30850374)) pipeline with the following parameters: fastqfiles=0.5, average quality=30, min quality=0. Briefly, each read was subjected to quality control, requiring an average base call quality greater than 30, and no ambiguous nucleotides. These filtered reads were aligned via the Needleman-Wunsch algorithm to their expected amplicon sequence, given their flanking primers, as extracted from human genome reference GRCh38. The results of the ampliCan pipeline were tallied and reported as the total edited and frameshift indels divided by the number of filtered reads passing quality control. Off-target editing rates for each enzyme were determined by targeted DNA sequencing of eight known off-target sites in total. The Cas9 editing and DNA sequencing were run in triplicate. For each site, the number of mutations induced by each enzyme were tallied and compared. To determine whether the average editing rate was different between the Cas9_wt and Cas9_R63A/Q768A enzymes, a t-test statistic was calculated.

TABLE 1
Plasmids for gene editing in eukaryotic cells

| | | |
|---|---|---|
| pEC2685 | CROPseq-Guide-Puro, EF1a-Puro-WPRE-hU6- | Gift from Christoph |
| | gRNA | Bock (Addgene |
| | | plasmid # 86708), |
| | | (Datlinger etal., 2017) |
| pEC2686 | pEC2685ΩEFS-NS-cas9(codon optimized), also | This study |
| | pCROPseqCas9_wt | |
| pEC2515 | pEC2686_cas9(R63A/Q768A) | This study |
| pEC2516 | pEC2686_cas9 (R66A/Q768A) | This study |
| pEC2526 | pEC2686_hU6::EpCAM1::gRNA | This study |
| pEC2527 | pEC2686_hU6::EpCAM2::gRNA | This study |
| pEC2528 | pEC2686_hU6::EpCAM3::gRNA | This study |
| pEC2529 | pEC2686_hU6::EpCAM4::gRNA | This study |
| pEC2530 | pEC2686_hU6::NTC1::gRNA | This study |
| pEC2531 | pEC2686_hU6::NTC2::gRNA | This study |
| pEC2532 | pEC2515_hU6::EpCAM1::gRNA | This study |
| pEC2533 | pEC2515_hU6::EpCAM2::gRNA | This study |
| pEC2534 | pEC2515_hU6::EpCAM3::gRNA | This study |
| pEC2535 | pEC2515_hU6::EpCAM4::gRNA | This study |
| pEC2536 | pEC2515_hU6::NTC1::gRNA | This study |
| pEC2537 | pEC2515_hU6::NTC2::gRNA | This study |
| pEC2538 | pEC2516_hU6::EpCAM1::gRNA | This study |
| pEC2539 | pEC2516_hU6::EpCAM2::gRNA | This study |
| pEC2540 | pEC2516_hU6::EpCAM3::gRNA | This study |
| pEC2541 | pEC2516_hU6::EpCAM4::gRNA | This study |
| pEC2542 | pEC2516_hU6::NTC1::gRNA | This study |
| pEC2543 | pEC2516_hU6::NTC2::gRNA | This study |
| pEC2626 | pEC2526_hU6::EpCAM1(MM13)::gRNA | This study |
| pEC2627 | pEC2526_hU6::EpCAM1(MM14)::gRNA | This study |
| pEC2629 | pEC2526_hU6::EpCAM1(MM16)::gRNA | This study |
| pEC2630 | pEC2526_hU6::EpCAM1(MM17)::gRNA | This study |
| pEC2631 | pEC2526_hU6::EpCAM1(MM18)::gRNA | This study |
| pEC2632 | pEC2526_hU6::EpCAM1(MM19)::gRNA | This study |
| pEC2636 | pEC2526_hU6::EpCAM4(MM13)::gRNA | This study |
| pEC2638 | pEC2526_hU6::EpCAM4(MM15)::gRNA | This study |
| pEC2639 | pEC2526_hU6::EpCAM4(MM16)::gRNA | This study |
| pEC2640 | pEC2526_hU6::EpCAM4(MM17)::gRNA | This study |
| pEC2641 | pEC2526_hU6::EpCAM4(MM18)::gRNA | This study |
| pEC2642 | pEC2526_hU6::EpCAM4(MM19)::gRNA | This study |
| pEC2646 | pEC2515_hU6::EpCAM1(MM13)::gRNA | This study |
| pEC2647 | pEC2515_hU6::EpCAM1(MM14)::gRNA | This study |
| pEC2649 | pEC2515_hU6::EpCAM1(MM16)::gRNA | This study |
| pEC2650 | pEC2515_hU6::EpCAM1(MM17)::gRNA | This study |
| pEC2651 | pEC2515_hU6::EpCAM1(MM18)::gRNA | This study |
| pEC2652 | pEC2515_hU6::EpCAM1(MM19)::gRNA | This study |
| pEC2656 | pEC2515_hU6::EpCAM4(MM13)::gRNA | This study |
| pEC2658 | pEC2515_hU6::EpCAM4(MM15)::gRNA | This study |
| pEC2659 | pEC2515_hU6::EpCAM4(MM16)::gRNA | This study |
| pEC2660 | pEC2515_hU6::EpCAM4(MM17)::gRNA | This study |
| pEC2661 | pEC2515_hU6::EpCAM4(MM18)::gRNA | This study |
| pEC2662 | pEC2515_hU6::EpCAM4(MM19)::gRNA | This study |
| p1490 | pEC2686_hU6::VEGFA1::gRNA | This study |
| p1491 | pEC2686_hU6::VEGFA3::gRNA | This study |
| p1492 | pEC2686_hU6::EMX1.4::gRNA | This study |
| p1498 | pEC2515_hU6::VEGFA1::gRNA | This study |
| p1499 | pEC2515_hU6::VEGFA3::gRNA | This study |
| p1500 | pEC2515_hU6::EMX1.4::gRNA | This study |

TABLE 2
Primers for PCR amplification of on- and off-target sites

| VEGFA1 | OLEC105 | TCCAGATGGCACATTGTCAG | F | Nested |
|---|---|---|---|---|
| on-target | 39 | (SEQ ID NO: 96) | | PCR |
| | OLEC105 | AGGGAGCAGGAAAGTGAGGT | R | |
| | 40 | (SEQ ID NO: 97) | | |
| VEGFA1 | OLEC104 | TCCAACGCCCTCAACCCCAC | F | PCR |
| on-target | 24 | (SEQ ID NO: 98) | | |
| | OLEC104 | CACACTGTGGCCCCTGTGC | R | |
| | 25 | (SEQ ID NO: 99) | | |
| VEGFA1 | OLEC104 | CAGCAGGACTGTGTGGCAC | F | PCR |
| OT1-6 | 26 | (SEQ ID NO: 100) | | |
| | OLEC104 | TCCTTCCAGCGATCCCATGG | R | |
| | 27 | (SEQ ID NO: 101) | | |
| VEGFA1 | OLEC104 | GCCCATTCTTTTTGCAGTGGA | F | PCR |
| OT1-11 | 32 | (SEQ ID NO: 102) | | |
| | OLEC104 | GAGAGCAAGTTTGTTCCCCAGG | R | |
| | 33 | (SEQ ID NO: 103) | | |
| EMX1.4 | OLEC104 | ATGGGAGCAGCTGGTCAGAG | F | PCR |
| on-target | 34 | (SEQ ID NO: 104) | | |
| | OLEC104 | TGGTTGCCCACCCTAGTCAT | R | |
| | 35 | (SEQ ID NO: 105) | | |
| EMX1.4 | OLEC104 | CAAGCTTTTCCTGACGCCCC | F | PCR |
| OT4-1 | 36 | (SEQ ID NO: 106) | | |
| | OLEC104 | TCCTGAAGACCTGTAATCTGACTCT | R | |
| | 37 | (SEQ ID NO: 107) | | |
| EMX1.4 | OLEC104 | GTGACTTGTTCCTGGTTCTGCC | F | PCR |
| OT4-52 | 38 | (SEQ ID NO: 108) | | |
| | OLEC104 | AGCTGTCCTGTCTCATTGGCT | R | |
| | 39 | (SEQ ID NO: 109) | | |
| EMX1.4 | OLEC104 | TGAAATCTCACCTGGGCGAGA | F | PCR |
| OT4-53 | 40 | (SEQ ID NO: 110) | | |
| | OLEC104 | TGCAGTCTGCCTTTTTGGGG | R | |
| | 41 | (SEQ ID NO: 111) | | |
| VEGFA3 | OLEC104 | CTGGGTGAATGGAGCGAGCAG | F | PCR |
| on-target | 42 | (SEQ ID NO: 112) | | |
| | OLEC104 | GCATTGGCGAGGAGGGAGCA | R | |
| | 43 | (SEQ ID NO: 113) | | |
| VEGFA3 | OLEC104 | TCTGTCACCACACAGTTACCACC | F | PCR |
| OT3-18 | 44 | (SEQ ID NO: 114) | | |
| | OLEC104 | GTTGCCTGGGGATGGGGTAT | R | |
| | 45 | (SEQ ID NO: 115) | | |
| VEGFA3 | OLEC104 | TCCTTTGAGGTTCATCCCCC | F | PCR |
| OT3-4 | 46 | (SEQ ID NO: 116) | | |
| | OLEC104 | CCAATCCAGGATGATTCCGC | R | |
| | 47 | (SEQ ID NO: 117) | | |
| VEGFA3 | OLEC104 | GAGGGGGAAGTCACCGACAA | F | PCR |
| OT3-2 | 48 | (SEQ ID NO: 118) | | |
| | OLEC104 | TACCCGGGCCGTCTGTTAGA | R | |
| | 49 | (SEQ ID NO: 119) | | |

TABLE 3
Cell lines used in the study.

| Cell line | Relevant characteristics | Source |
|---|---|---|
| MCF-7 | Human breast cancer cells, epithelial | ATCC HTB-22 ™ |
| HEK293 | Human embryonic kidney cells, | ATCC CRL-1573 ™ |
| | epithelial | |

### Example 8

Cas9_R63A/Q768A Enhances Specificity of Human Gene Editing

To assess whether Cas9_R63A/Q768A (i.e. the Cas9 variant that was demonstrated to possess improved specificity in vitro and in bacteria) is active in human cells, gene editing experiments in the human breast cancer cell line MCF-7 were performed with four different sgRNAs targeting EpCAM for deletion. It was decided to select EpCAM due to its function as an oncogene and its potential as relevant clinical target (Munch, Nat Communications 10.6, 2015 (PMID:25665714)); Münz, Oncogene. 2004 Jul. 29; 23(34):5748-58 (PMID 15195135); and Armstrong, Cancer Biol Ther. 2003 July-August; 2(4):320-6 (PMID 14508099)). In several cancer cell lines, EpCAM expression is strongly upregulates (Balzar, J Mol Med (Berl). 1999 October; 77(10):699-712 (PMID 10606205)) and siRNA-dependent silencing of EpCAM in vitro led to decreased proliferation, migration, and invasion of breast cancer cells (Osta, Cancer Res. 2004 Aug. 15; 64(16):5818-24 (PMID 15313925)).

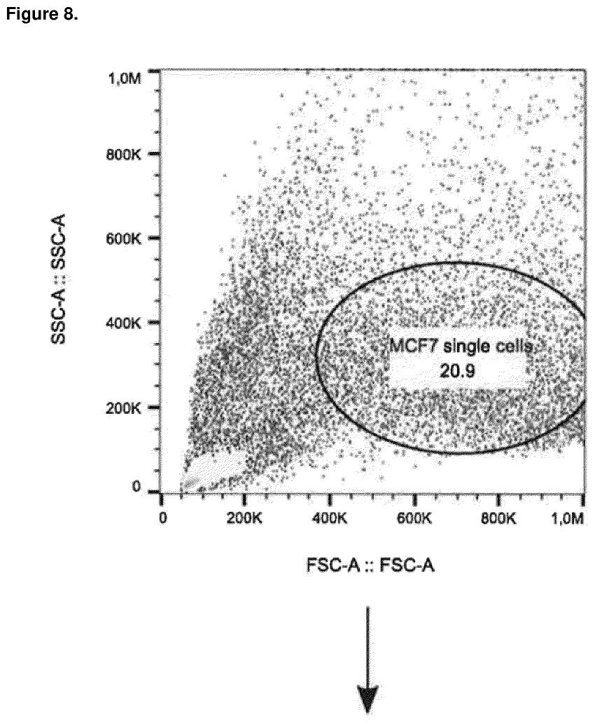

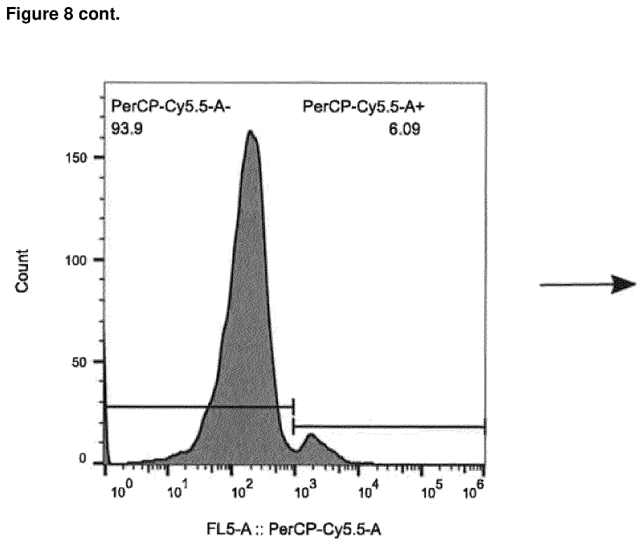

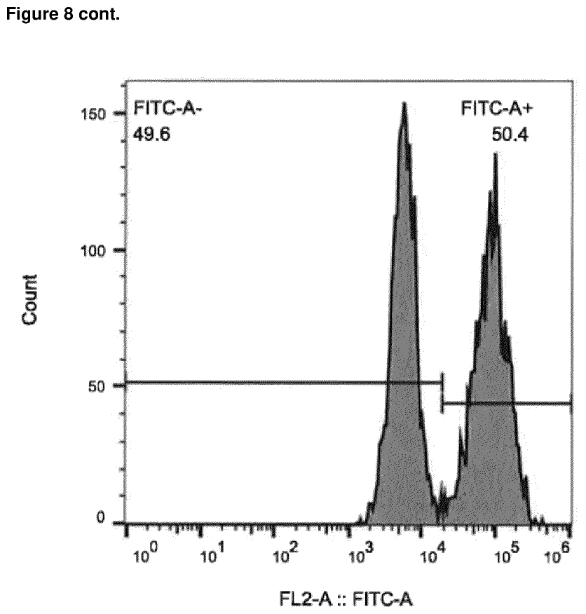

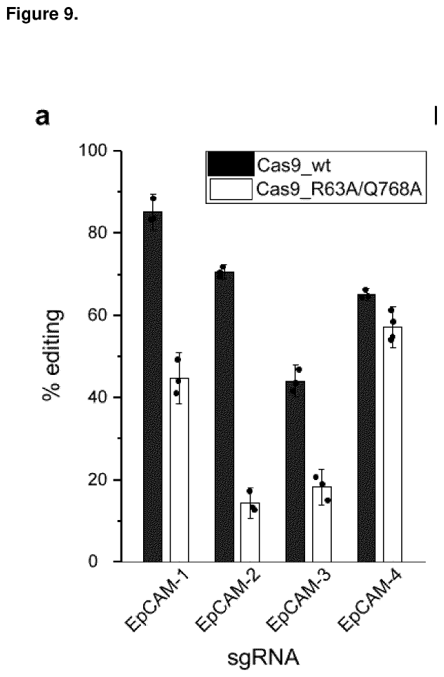

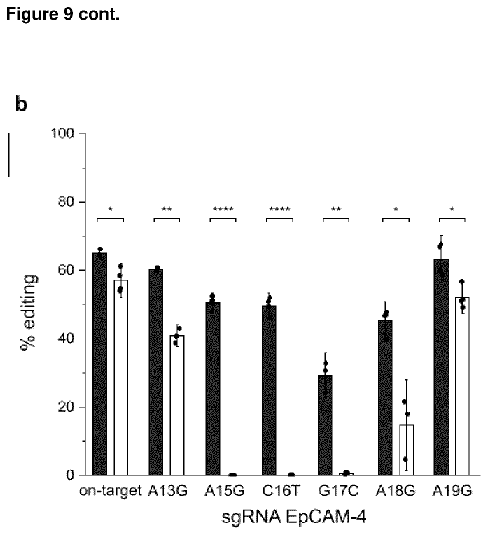

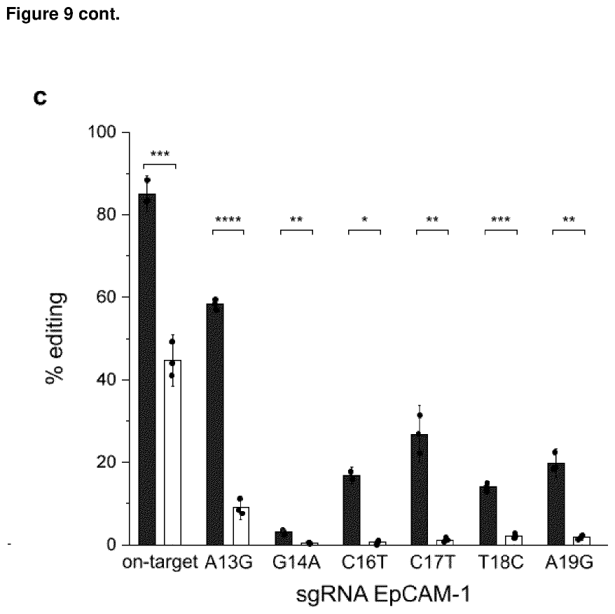

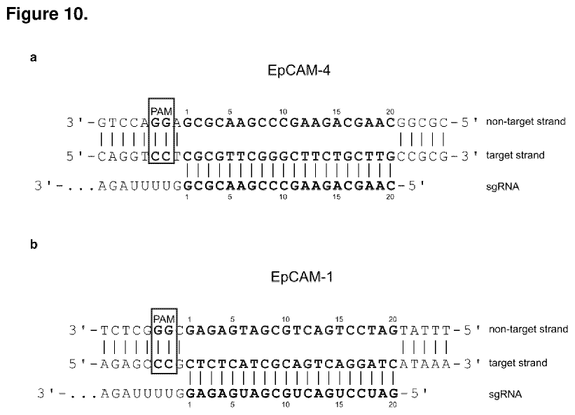

Flow cytometry was used to determine the fraction of EpCAM^{positive} versus EpCAM^{negative} cells (FIG. 8) as a read out for gene deletion efficiency at single cell resolution. Cas9_R63A/Q768A showed activity, although at lower frequency than Cas9_wt (FIG. 9a). To test whether Cas9_R63A/Q768A is more sensitive to mismatches than Cas9_wt, point mutations were introduced in sgRNAs EpCAM-1 and EpCAM-4, which resulted in a mismatch to these target sites in several PAM-distal positions (FIG. 10) and the level of EpCAM editing was determined. Cas9_R63A/Q768A showed increased specificity in the presence of mismatches in a sgRNA dependent manner. In the presence of sgRNA EpCAM-4, Cas9_R63A/Q768A was more sensitive to most PAM-distal mismatches when compared to Cas9_wt (FIG. 9b). In the presence of sgRNA EpCAM-1, minimal to no editing was observed for positions 14 and 16-19 with Cas9_R63A/Q768A, which is probably due to a difference in the on-target activity between Cas9_wt and the Cas9 variant. Considering this reduced on target activity, Cas9_R63A/Q768A was significantly more specific than Cas9_wt on position 13 (FIG. 9c). Notably, no editing by Cas9_R63A/Q768A in the presence of a mismatch in position 15 was observed, which is in good agreement with the results obtained in vitro and in bacterial survival assays (see Example 5). In Summary, Cas9_R63A/Q768A showed enhanced specificity to both tested sgRNA in human cells.

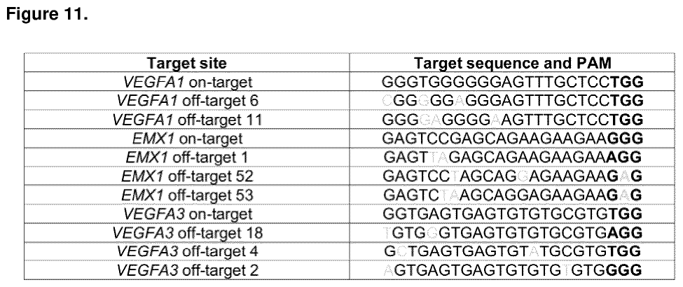

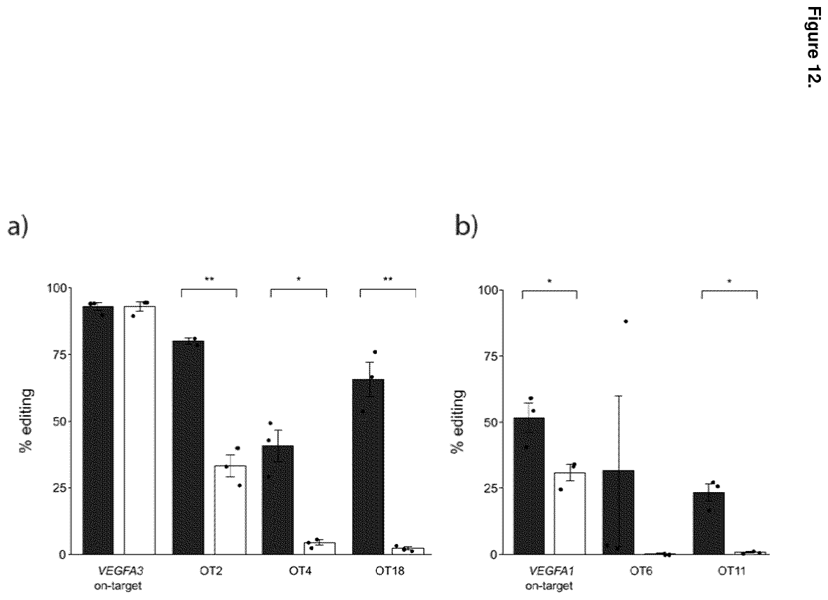

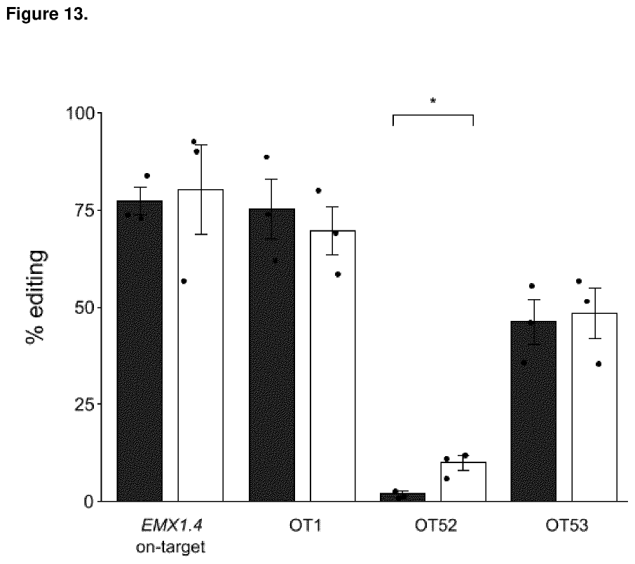

Gene editing experiments were performed in HEK293 cells with two sgRNAs targeting VEGFA and one sgRNA targeting EMX1 with previously characterized off-target sites (Fu, Y., Sander, J. D., Reyon, D., Cascio, V. M. & Joung, J. K. Improving CRISPR-Cas nuclease specificity using truncated guide RNAs. Nat Biotech. 32, 279 (2014)). These two sites were chosen because 1) sgRNAs for these genes have been well characterized for off-target sites and 2) Increased expression of VEGFA is correlated with tumor development and thus VEGFA is considered as a relevant target for novel cancer treatment strategies (Stockmann, Nature. 2008 Dec. 11; 456(7223):814-8 (PMID 18997773). On- and off-target sites (FIG. 11) from Cas9-treated cells were PCR amplified and subjected to amplicon sequencing achieving a mean coverage of 64 thousand paired-end reads per library. Cas9_R63A/Q768A was able to cleave the on-target sites with all three guides with similar (VEGFA3 and EMX1.4) or lower (VEGFA1) editing efficiency than Cas9_wt, in agreement with the results obtained using sgRNAs targeting EpCAM (FIG. 9). Importantly, Cas9_R63A/Q768A showed significantly higher specificity at off-target sites compared to Cas9_wt (FIG. 12), except for the sgRNA targeting MX/(FIG. 13).

Thus, the results show that Cas9_R63A/Q768A displays enhanced specificity at certain off-target sites in human cells.

CRISPR-Cas9 has become the method of choice for a variety of gene targeting and engineering applications. Hence, designing highly specific Cas9 variants that do not recognize and cleave off-target sequences in eukaryotic cells is of critical importance. Considering its natural function as a defense system against invading nucleic acids, such as bacteriophages, the native Cas9 enzymes had to evolve to tolerate certain mismatches and still be able to cleave viral escape mutants (Datsenko, Nat Commun. 2012 Jul. 10; 3:945 (PMID:22781758)). Cas9 is sensitive to mismatches in the PAM-adjacent and the PAM-distal part of the target but shows certain flexibility towards mismatches if they are located in the middle of the target sequence (Jinek, Science. 2012 Aug. 17; 337(6096):816-21 (PMID:22745249)). Here a Cas9 variant, namely Cas9_R63A/Q768A, was created that displays increased specificity in human cells. It was demonstrated that Cas9_R63A/Q768A is active in different human cell lines, thereby showing improved sensitivity to mismatches for sgRNAs targeting different genes.

Although it could be shown that Cas9_R63A/Q768A displays increased specificity for different sgRNAs targeting different genes, it was also observed that for one specific sgRNA Cas9_R63A/Q768A has a slightly decreased specificity when compared to Cas9 WT. It is well known since the beginning of Cas9 application that the sequence of the sgRNA alone can affect specificity independent of Cas9 features (Wu, Quant Biol. 2014 June; 2(2):59-70 (PMID: 25722925)). Although this effect has been described for a long time, it is still poorly understood and several mechanisms have been proposed.

Due to the poorly understood sequence-dependent effect of the sgRNA on off-target cleavage, it is suggested herein to complement the established computational tools (Labun, Nucleic Acids Res. 2016 Jul. 8; 44(W1):W272-6 (PMID 27185894); Haeussler; Genome Biol. 2016 Jul. 5; 17(1):148 (PMID 27380939)) that predict the “perfect” sgRNA with further experimental steps for validating the selected sgRNA. Hence, additional experimental steps, such as whole-genome sequencing or double stranded break capture are highly beneficial for defining the ideal sgRNA that can be considered safe for therapeutic applications. In this approach it is feasible to test several Cas9 variants for their specificity and the herewith provided results indicate that Cas9_R63A/Q768A is more specific for the majority of sgRNAs and thus should be considered instead of Cas9 WT for biomedical applications.

In summary, it has been identified that two distinct residues together influence Cas9 specificity. Interestingly, replacement of R63 and Q768 with alanine residues enhances Cas9 specificity compared to wt Cas9. Off-target cleavage has been reported for Cas9, and might lead to additional, undesired mutations. The herein provided Cas9 variants with enhanced specificity therefore represent a means of improving the Cas9 genome editing technology for applications in life science research, biotechnology, agriculture and medicine.

The present invention refers to the following nucleotide and amino acid sequences:

Wild type Cas9 of Streptococcus pyrogenes (SpCas9) amino acid sequence;
SEQ ID NO: 1
MDKKYSIGLDIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD
Wild type SpCas9 amino acid sequence wherein the arginine (R) at position 63
and the glutamine (Q) at position 768 are each replaced by alanine (A);
SEQ ID NO: 2
MDKKYSIGLDIGTNSVGWAVITDEYKVPSKKFKVLGNTDRHSIKKNLIGALLFDSGETAE
ATALKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENATTQKGQKNSRER
MKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD
Wild type SpCas9 amino acid sequence wherein the arginine (R) at position 66
and the glutamine (Q) at position 768 are each replaced by alanine (A);
SEQ ID NO: 3
MDKKYSIGLDIGTNSVGWAVITDEYKVPSKKFKVLGNTDRHSIKKNLIGALLFDSGETAE
ATRLKATARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENATTQKGQKNSRER
MKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD
Sequence for S pyogenes Cas9 (including N-terminal His-tag), recombinant
expressed in E. coli, purified and used for in vitro experiments; SEQ ID NO: 4:
ATGGGCCATCATCATCATCATCATCATCATCATCACAGCAGCGGCCATATCGAAGGTCGT
CATAGCGTCGACATGGATAAGAAATACTCAATAGGCTTAGATATCGGCACAAATAGCGTC
GGATGGGCGGTGATCACTGATGAATATAAGGTTCCGTCTAAAAAGTTCAAGGTTCTGGGA
AATACAGACCGCCACAGTATCAAAAAAAATCTTATAGGGGCTCTTTTATTTGACAGTGGA
GAGACAGCGGAAGCGACTCGTCTCAAACGGACAGCTCGTAGAAGGTATACACGTCGGAA
GAATCGTATTTGTTATCTACAGGAGATTTITTCAAATGAGATGGCGAAAGTAGATGATAG
TTTCTTTCATCGACTTGAAGAGTCTTTTTTGGTGGAAGAAGACAAGAAGCATGAACGTCA
TCCTATTTTTGGAAATATAGTAGATGAAGTTGCTTATCATGAGAAATATCCAACTATCTAT
CATCTGCGAAAAAAATTGGTAGATTCTACTGATAAAGCGGATTTGCGCTTAATCTATTTG
GCCTTAGCGCATATGATTAAGTTTCGTGGTCATTTTTTGATTGAGGGAGATTTAAATCCTG
ATAATAGTGATGTGGACAAACTATTTATCCAGTTGGTACAAACCTACAATCAATTATTTG
AAGAAAACCCTATTAACGCAAGTGGAGTAGATGCTAAAGCGATTCTTTCTGCACGATTGA
GTAAATCAAGACGATTAGAAAATCTCATTGCTCAGCTCCCCGGTGAGAAGAAAAATGGCT
TATTTGGGAATCTCATTGCTTTGTCATTGGGTTTGACCCCTAATTTTAAATCAAATTTTGAT
TTGGCAGAAGATGCTAAATTACAGCTTTCAAAAGATACTTACGATGATGATTTAGATAAT
TTATTGGCGCAAATTGGAGATCAATATGCTGATTTGTTTTTGGCAGCTAAGAATTTATCAG
ATGCTATTTTACTTTCAGATATCCTAAGAGTAAATACTGAAATAACTAAGGCTCCCCTATC
AGCTTCAATGATTAAACGCTACGATGAACATCATCAAGACTTGACTCTTTTAAAAGCTTT
AGTTCGACAACAACTTCCAGAAAAGTATAAAGAAATCTTTTTTGATCAATCAAAAAACGG
ATATGCAGGTTATATTGATGGGGGAGCTAGCCAAGAAGAATTTTATAAATTTATCAAACC
AATTTTAGAAAAAATGGATGGTACTGAGGAATTATTGGTGAAACTAAATCGTGAAGATTT
GCTGCGCAAGCAACGGACCTTTGACAACGGCTCTATTCCCCATCAAATTCACTTGGGTGA
GCTGCATGCTATTTTGAGAAGACAAGAAGACTTTTATCCATTTTTAAAAGACAATCGTGA
GAAGATTGAAAAAATCTTGACTTTTCGAATTCCTTATTATGTTGGTCCATTGGCGCGTGGC
AATAGTCGTTTTGCATGGATGACTCGGAAGTCTGAAGAAACAATTACCCCATGGAATTTT
GAAGAAGTTGTCGATAAAGGTGCTTCAGCTCAATCATTTATTGAACGCATGACAAACTTT
GATAAAAATCTTCCAAATGAAAAAGTACTACCAAAACATAGTTTGCTTTATGAGTATTTT
ACGGTTTATAACGAATTGACAAAGGTCAAATATGTTACTGAAGGAATGCGAAAACCAGC
ATTTCTTTCAGGTGAACAGAAGAAAGCCATTGTTGATTTACTCTTCAAAACAAATCGAAA
AGTAACCGTTAAGCAATTAAAAGAAGATTATTTCAAAAAAATAGAATGTTTTGATAGTGT
TGAAATTTCAGGAGTTGAAGATAGATTTAATGCTTCATTAGGTACCTACCATGATTTGCTA
AAAATTATTAAAGATAAAGATTTTTTGGATAATGAAGAAAATGAAGATATCTTAGAGGAT
ATTGTTTTAACATTGACCTTATTTGAAGATAGGGAGATGATTGAGGAAAGACTTAAAACA
TATGCTCACCTCTTTGATGATAAGGTGATGAAACAGCTTAAACGTCGCCGTTATACTGGTT
GGGGACGTTTGTCTCGAAAATTGATTAATGGTATTAGGGATAAGCAATCTGGCAAAACAA
TATTAGATTTTTTGAAATCAGATGGTTTTGCCAATCGCAATTTTATGCAGCTGATCCATGA
TGATAGTTTGACATTTAAAGAAGACATTCAAAAAGCACAAGTGTCTGGACAAGGCGATA
GTTTACATGAACATATTGCAAATTTAGCTGGTAGCCCTGCTATTAAAAAAGGTATTTTACA
GACTGTAAAAGTTGTTGATGAATTGGTCAAAGTAATGGGGCGGCATAAGCCAGAAAATA
TCGTTATTGAAATGGCACGTGAAAATCAGACAACTCAAAAGGGCCAGAAAAATTCGCGA
GAGCGTATGAAACGAATCGAAGAAGGTATCAAAGAATTAGGAAGTCAGATTCTTAAAGA
GCATCCTGTTGAAAATACTCAATTGCAAAATGAAAAGCTCTATCTCTATTATCTCCAAAAT
GGAAGAGACATGTATGTGGACCAAGAATTAGATATTAATCGTTTAAGTGATTATGATGTC
GATCACATTGTTCCACAAAGTTTCCTTAAAGACGATTCAATAGACAATAAGGTCTTAACG
CGTTCTGATAAAAATCGTGGTAAATCGGATAACGTTCCAAGTGAAGAAGTAGTCAAAAA
GATGAAAAACTATTGGAGACAACTTCTAAACGCCAAGTTAATCACTCAACGTAAGTTTGA
TAATTTAACGAAAGCTGAACGTGGAGGTTTGAGTGAACTTGATAAAGCTGGTTTTATCAA
ACGCCAATTGGTTGAAACTCGCCAAATCACTAAGCATGTGGCACAAATTTTGGATAGTCG
CATGAATACTAAATACGATGAAAATGATAAACTTATTCGAGAGGTTAAAGTGATTACCTT
AAAATCTAAATTAGTTTCTGACTTCCGAAAAGATTTCCAATTCTATAAAGTACGTGAGATT
AACAATTACCATCATGCCCATGATGCGTATCTAAATGCCGTCGTTGGAACTGCTTTGATTA
AGAAATATCCAAAACTTGAATCGGAGTTTGTCTATGGTGATTATAAAGTTTATGATGTTC
GTAAAATGATTGCTAAGTCTGAGCAAGAAATAGGCAAAGCAACCGCAAAATATTTCTTTT
ACTCTAATATCATGAACTTCTTCAAAACAGAAATTACACTTGCAAATGGAGAGATTCGCA
AACGCCCTCTAATCGAAACTAATGGGGAAACTGGAGAAATTGTCTGGGATAAAGGGCGA
GATTTTGCCACAGTGCGCAAAGTATTGTCCATGCCCCAAGTCAATATTGTCAAGAAAACA
GAAGTACAGACAGGCGGATTCTCCAAGGAGTCAATTTTACCAAAAAGAAATTCGGACAA
GCTTATTGCTCGTAAAAAAGACTGGGATCCAAAAAAATATGGTGGTTTTGATAGTCCAAC
GGTAGCTTATTCAGTCCTAGTGGTTGCTAAGGTGGAAAAAGGGAAATCGAAGAAGTTAA
AATCCGTTAAAGAGTTACTAGGGATCACAATTATGGAAAGAAGTTCCTTTGAAAAAAATC
CGATTGACTTTTTAGAAGCTAAAGGATATAAGGAAGTTAAAAAAGACTTAATCATTAAAC
TACCTAAATATAGTCTTTTTGAGTTAGAAAACGGTCGTAAACGGATGCTGGCTAGTGCCG
GAGAATTACAAAAAGGAAATGAGCTGGCTCTGCCAAGCAAATATGTGAATTTTTTATATT
TAGCTAGTCATTATGAAAAGTTGAAGGGTAGTCCAGAAGATAACGAACAAAAACAATTG
TTTGTGGAGCAGCATAAGCATTATTTAGATGAGATTATTGAGCAAATCAGTGAATTTTCT
AAGCGTGTTATTTTAGCAGATGCCAATTTAGATAAAGTTCTTAGTGCATATAACAAACAT
AGAGACAAACCAATACGTGAACAAGCAGAAAATATTATTCATTTATTTACGTTGACGAAT
CTTGGAGCTCCCGCTGCTTTTAAATATTTTGATACAACAATTGATCGTAAACGATATACGT
CTACAAAAGAAGTTTTAGATGCCACTCTTATCCATCAATCCATCACTGGTCTTTATGAAAC
ACGCATTGATTTGAGTCAGCTAGGAGGTGACTGA
Sequence for S. pyogenes Cas9_R63A (including N-terminal His-tag), recombinant
expressed in E.coli, purified and used for in vitro experiments; SEQ ID NO: 5:
ATGGGCCATCATCATCATCATCATCATCATCATCACAGCAGCGGCCATATCGAAGGTCGT
CATAGCGTCGACATGGATAAGAAATACTCAATAGGCTTAGATATCGGCACAAATAGCGTC
GGATGGGCGGTGATCACTGATGAATATAAGGTTCCGTCTAAAAAGTTCAAGGTTCTGGGA
AATACAGACCGCCACAGTATCAAAAAAAATCTTATAGGGGCTCTTTTATTTGACAGTGGA
GAGACAGCGGAAGCGACTGCTCTCAAACGGACAGCTCGTAGAAGGTATACACGTCGGAA
GAATCGTATTTGTTATCTACAGGAGATTTTTTCAAATGAGATGGCGAAAGTAGATGATAG
TTTCTTTCATCGACTTGAAGAGTCTTTTTTGGTGGAAGAAGACAAGAAGCATGAACGTCA
TCCTATTTTTGGAAATATAGTAGATGAAGTTGCTTATCATGAGAAATATCCAACTATCTAT
CATCTTCGAAAAAAATTGGTAGATTCTACTGATAAAGCGGATTTGCGCTTAATCTATTTGG
CCTTAGCGCATATGATTAAGTTTCGTGGTCATTTTTTGATTGAGGGAGATTTAAATCCTGA
TAATAGTGATGTGGACAAACTATTTATCCAGTTGGTACAAACCTACAATCAATTATTTGA
AGAAAACCCTATTAACGCAAGTGGAGTAGATGCTAAAGCGATTCTTTCTGCACGATTGAG
TAAATCAAGACGATTAGAAAATCTCATTGCTCAGCTCCCCGGTGAGAAGAAAAATGGCTT
ATTTGGGAATCTCATTGCTTTGTCATTGGGTTTGACCCCTAATTTTAAATCAAATTTTGATT
TGGCAGAAGATGCTAAATTACAGCTTTCAAAAGATACTTACGATGATGATTTAGATAATT
TATTGGCGCAAATTGGAGATCAATATGCTGATTTGTTTTTGGCAGCTAAGAATTTATCAGA
TGCTATTTTACTTTCAGATATCCTAAGAGTAAATACTGAAATAACTAAGGCTCCCCTATCA
GCTTCAATGATTAAACGCTACGATGAACATCATCAAGACTTGACTCTTTTAAAAGCTTTA
GTTCGACAACAACTTCCAGAAAAGTATAAAGAAATCTTTTTTGATCAATCAAAAAACGGA
TATGCAGGTTATATTGATGGGGGAGCTAGCCAAGAAGAATTTTATAAATTTATCAAACCA
ATTTTAGAAAAAATGGATGGTACTGAGGAATTATTGGTGAAACTAAATCGTGAAGATTTG
CTGCGCAAGCAACGGACCTTTGACAACGGCTCTATTCCCCATCAAATTCACTTGGGTGAG
CTGCATGCTATTTTGAGAAGACAAGAAGACTTTTATCCATTTTTAAAAGACAATCGTGAG
AAGATTGAAAAAATCTTGACTTTTCGAATTCCTTATTATGTTGGTCCATTGGCGCGTGGCA
ATAGTCGTTTTGCATGGATGACTCGGAAGTCTGAAGAAACAATTACCCCATGGAATTTTG
AAGAAGTTGTCGATAAAGGTGCTTCAGCTCAATCATTTATTGAACGCATGACAAACTTTG
ATAAAAATCTTCCAAATGAAAAAGTACTACCAAAACATAGTTTGCTTTATGAGTATTTTA
CGGTTTATAACGAATTGACAAAGGTCAAATATGTTACTGAAGGAATGCGAAAACCAGCA
TTTCTTTCAGGTGAACAGAAGAAAGCCATTGTTGATTTACTCTTCAAAACAAATCGAAAA
GTAACCGTTAAGCAATTAAAAGAAGATTATTTCAAAAAAATAGAATGTTTTGATAGTGTT
GAAATTTCAGGAGTTGAAGATAGATTTAATGCTTCATTAGGTACCTACCATGATTTGCTA
AAAATTATTAAAGATAAAGATTTTTTGGATAATGAAGAAAATGAAGATATCTTAGAGGAT
ATTGTTTTAACATTGACCTTATTTGAAGATAGGGAGATGATTGAGGAAAGACTTAAAACA
TATGCTCACCTCTTTGATGATAAGGTGATGAAACAGCTTAAACGTCGCCGTTATACTGGTT
GGGGACGTTTGTCTCGAAAATTGATTAATGGTATTAGGGATAAGCAATCTGGCAAAACAA
TATTAGATTTTTTGAAATCAGATGGTTTTGCCAATCGCAATTTTATGCAGCTGATCCATGA
TGATAGTTTGACATTTAAAGAAGACATTCAAAAAGCACAAGTGTCTGGACAAGGCGATA
GTTTACATGAACATATTGCAAATTTAGCTGGTAGCCCTGCTATTAAAAAAGGTATTTTACA
GACTGTAAAAGTTGTTGATGAATTGGTCAAAGTAATGGGGCGGCATAAGCCAGAAAATA
TCGTTATTGAAATGGCACGTGAAAATCAGACAACTCAAAAGGGCCAGAAAAATTCGCGA
GAGCGTATGAAACGAATCGAAGAAGGTATCAAAGAATTAGGAAGTCAGATTCTTAAAGA
GCATCCTGTTGAAAATACTCAATTGCAAAATGAAAAGCTCTATCTCTATTATCTCCAAAAT
GGAAGAGACATGTATGTGGACCAAGAATTAGATATTAATCGTTTAAGTGATTATGATGTC
GATCACATTGTTCCACAAAGTTTCCTTAAAGACGATTCAATAGACAATAAGGTCTTAACG
CGTTCTGATAAAAATCGTGGTAAATCGGATAACGTTCCAAGTGAAGAAGTAGTCAAAAA
GATGAAAAACTATTGGAGACAACTTCTAAACGCCAAGTTAATCACTCAACGTAAGTTTGA
TAATTTAACGAAAGCTGAACGTGGAGGTTTGAGTGAACTTGATAAAGCTGGTTTTATCAA
ACGCCAATTGGTTGAAACTCGCCAAATCACTAAGCATGTGGCACAAATTTTGGATAGTCG
CATGAATACTAAATACGATGAAAATGATAAACTTATTCGAGAGGTTAAAGTGATTACCTT
AAAATCTAAATTAGTTTCTGACTTCCGAAAAGATTTCCAATTCTATAAAGTACGTGAGATT
AACAATTACCATCATGCCCATGATGCGTATCTAAATGCCGTCGTTGGAACTGCTTTGATTA
AGAAATATCCAAAACTTGAATCGGAGTTTGTCTATGGTGATTATAAAGTTTATGATGTTC
GTAAAATGATTGCTAAGTCTGAGCAAGAAATAGGCAAAGCAACCGCAAAATATTTCTTTT
ACTCTAATATCATGAACTTCTTCAAAACAGAAATTACACTTGCAAATGGAGAGATTCGCA
AACGCCCTCTAATCGAAACTAATGGGGAAACTGGAGAAATTGTCTGGGATAAAGGGCGA
GATTTTGCCACAGTGCGCAAAGTATTGTCCATGCCCCAAGTCAATATTGTCAAGAAAACA
GAAGTACAGACAGGCGGATTCTCCAAGGAGTCAATTTTACCAAAAAGAAATTCGGACAA
GCTTATTGCTCGTAAAAAAGACTGGGATCCAAAAAAATATGGTGGTTTTGATAGTCCAAC
GGTAGCTTATTCAGTCCTAGTGGTTGCTAAGGTGGAAAAAGGGAAATCGAAGAAGTTAA
AATCCGTTAAAGAGTTACTAGGGATCACAATTATGGAAAGAAGTTCCTTTGAAAAAAATC
CGATTGACTTTTTAGAAGCTAAAGGATATAAGGAAGTTAAAAAAGACTTAATCATTAAAC
TACCTAAATATAGTCTTTTTGAGTTAGAAAACGGTCGTAAACGGATGCTGGCTAGTGCCG
GAGAATTACAAAAAGGAAATGAGCTGGCTCTGCCAAGCAAATATGTGAATTTTTTATATT
TAGCTAGTCATTATGAAAAGTTGAAGGGTAGTCCAGAAGATAACGAACAAAAACAATTG
TTTGTGGAGCAGCATAAGCATTATTTAGATGAGATTATTGAGCAAATCAGTGAATTTTCT
AAGCGTGTTATTTTAGCAGATGCCAATTTAGATAAAGTTCTTAGTGCATATAACAAACAT
AGAGACAAACCAATACGTGAACAAGCAGAAAATATTATTCATTTATTTACGTTGACGAAT
CTTGGAGCTCCCGCTGCTTTTAAATATTTTGATACAACAATTGATCGTAAACGATATACGT
CTACAAAAGAAGTTTTAGATGCCACTCTTATCCATCAATCCATCACTGGTCTTTATGAAAC
ACGCATTGATTTGAGTCAGCTAGGAGGTGACTGA
Sequence for S. pyogenes Cas9_R66A (including N-terminal His-tag), recombinant
expressed in E.coli, purified and used for in vitro experiments; SEQ ID NO: 6:
ATGGGCCATCATCATCATCATCATCATCATCATCACAGCAGCGGCCATATCGAAGGTCGT
CATAGCGTCGACATGGATAAGAAATACTCAATAGGCTTAGATATCGGCACAAATAGCGTC
GGATGGGCGGTGATCACTGATGAATATAAGGTTCCGTCTAAAAAGTTCAAGGTTCTGGGA
AATACAGACCGCCACAGTATCAAAAAAAATCTTATAGGGGCTCTTTTATTTGACAGTGGA
GAGACAGCGGAAGCGACTCGTCTCAAAGCGACAGCTCGTAGAAGGTATACACGTCGGAA
GAATCGTATTTGTTATCTACAGGAGATTTTTTCAAATGAGATGGCGAAAGTAGATGATAG
TTTCTTTCATCGACTTGAAGAGTCTTTTTTGGTGGAAGAAGACAAGAAGCATGAACGTCA
TCCTATTTTTGGAAATATAGTAGATGAAGTTGCTTATCATGAGAAATATCCAACTATCTAT
CATCTTCGAAAAAAATTGGTAGATTCTACTGATAAAGCGGATTTGCGCTTAATCTATTTGG
CCTTAGCGCATATGATTAAGTTTCGTGGTCATTTTTTGATTGAGGGAGATTTAAATCCTGA
TAATAGTGATGTGGACAAACTATTTATCCAGTTGGTACAAACCTACAATCAATTATTTGA
AGAAAACCCTATTAACGCAAGTGGAGTAGATGCTAAAGCGATTCTTTCTGCACGATTGAG
TAAATCAAGACGATTAGAAAATCTCATTGCTCAGCTCCCCGGTGAGAAGAAAAATGGCTT
ATTTGGGAATCTCATTGCTTTGTCATTGGGTTTGACCCCTAATTTTAAATCAAATTTTGATT
TGGCAGAAGATGCTAAATTACAGCTTTCAAAAGATACTTACGATGATGATTTAGATAATT
TATTGGCGCAAATTGGAGATCAATATGCTGATTTGTTTTTGGCAGCTAAGAATTTATCAGA
TGCTATTTTACTTTCAGATATCCTAAGAGTAAATACTGAAATAACTAAGGCTCCCCTATCA
GCTTCAATGATTAAACGCTACGATGAACATCATCAAGACTTGACTCTTTTAAAAGCTTTA
GTTCGACAACAACTTCCAGAAAAGTATAAAGAAATCTTTTTTGATCAATCAAAAAACGGA
TATGCAGGTTATATTGATGGGGGAGCTAGCCAAGAAGAATTTTATAAATTTATCAAACCA
ATTTTAGAAAAAATGGATGGTACTGAGGAATTATTGGTGAAACTAAATCGTGAAGATTTG
CTGCGCAAGCAACGGACCTTTGACAACGGCTCTATTCCCCATCAAATTCACTTGGGTGAG
CTGCATGCTATTTTGAGAAGACAAGAAGACTTTTATCCATTTTTAAAAGACAATCGTGAG
AAGATTGAAAAAATCTTGACTTTTCGAATTCCTTATTATGTTGGTCCATTGGCGCGTGGCA
ATAGTCGTTTTGCATGGATGACTCGGAAGTCTGAAGAAACAATTACCCCATGGAATTTTG
AAGAAGTTGTCGATAAAGGTGCTTCAGCTCAATCATTTATTGAACGCATGACAAACTTTG
ATAAAAATCTTCCAAATGAAAAAGTACTACCAAAACATAGTTTGCTTTATGAGTATTTTA
CGGTTTATAACGAATTGACAAAGGTCAAATATGTTACTGAAGGAATGCGAAAACCAGCA
TTTCTTTCAGGTGAACAGAAGAAAGCCATTGTTGATTTACTCTTCAAAACAAATCGAAAA
GTAACCGTTAAGCAATTAAAAGAAGATTATTTCAAAAAAATAGAATGTTTTGATAGTGTT
GAAATTTCAGGAGTTGAAGATAGATTTAATGCTTCATTAGGTACCTACCATGATTTGCTA
AAAATTATTAAAGATAAAGATTTTTTGGATAATGAAGAAAATGAAGATATCTTAGAGGAT
ATTGTTTTAACATTGACCTTATTTGAAGATAGGGAGATGATTGAGGAAAGACTTAAAACA
TATGCTCACCTCTTTGATGATAAGGTGATGAAACAGCTTAAACGTCGCCGTTATACTGGTT
GGGGACGTTTGTCTCGAAAATTGATTAATGGTATTAGGGATAAGCAATCTGGCAAAACAA
TATTAGATTTTTTGAAATCAGATGGTTTTGCCAATCGCAATTTTATGCAGCTGATCCATGA
TGATAGTTTGACATTTAAAGAAGACATTCAAAAAGCACAAGTGTCTGGACAAGGCGATA
GTTTACATGAACATATTGCAAATTTAGCTGGTAGCCCTGCTATTAAAAAAGGTATTTTACA
GACTGTAAAAGTTGTTGATGAATTGGTCAAAGTAATGGGGCGGCATAAGCCAGAAAATA
TCGTTATTGAAATGGCACGTGAAAATCAGACAACTCAAAAGGGCCAGAAAAATTCGCGA
GAGCGTATGAAACGAATCGAAGAAGGTATCAAAGAATTAGGAAGTCAGATTCTTAAAGA
GCATCCTGTTGAAAATACTCAATTGCAAAATGAAAAGCTCTATCTCTATTATCTCCAAAAT
GGAAGAGACATGTATGTGGACCAAGAATTAGATATTAATCGTTTAAGTGATTATGATGTC
GATCACATTGTTCCACAAAGTTTCCTTAAAGACGATTCAATAGACAATAAGGTCTTAACG
CGTTCTGATAAAAATCGTGGTAAATCGGATAACGTTCCAAGTGAAGAAGTAGTCAAAAA
GATGAAAAACTATTGGAGACAACTTCTAAACGCCAAGTTAATCACTCAACGTAAGTTTGA
TAATTTAACGAAAGCTGAACGTGGAGGTTTGAGTGAACTTGATAAAGCTGGTTTTATCAA
ACGCCAATTGGTTGAAACTCGCCAAATCACTAAGCATGTGGCACAAATTTTGGATAGTCG
CATGAATACTAAATACGATGAAAATGATAAACTTATTCGAGAGGTTAAAGTGATTACCTT
AAAATCTAAATTAGTTTCTGACTTCCGAAAAGATTTCCAATTCTATAAAGTACGTGAGATT
AACAATTACCATCATGCCCATGATGCGTATCTAAATGCCGTCGTTGGAACTGCTTTGATTA
AGAAATATCCAAAACTTGAATCGGAGTTTGTCTATGGTGATTATAAAGTTTATGATGTTC
GTAAAATGATTGCTAAGTCTGAGCAAGAAATAGGCAAAGCAACCGCAAAATATTTCTTTT
ACTCTAATATCATGAACTTCTTCAAAACAGAAATTACACTTGCAAATGGAGAGATTCGCA
AACGCCCTCTAATCGAAACTAATGGGGAAACTGGAGAAATTGTCTGGGATAAAGGGCGA
GATTTTGCCACAGTGCGCAAAGTATTGTCCATGCCCCAAGTCAATATTGTCAAGAAAACA
GAAGTACAGACAGGCGGATTCTCCAAGGAGTCAATTTTACCAAAAAGAAATTCGGACAA
GCTTATTGCTCGTAAAAAAGACTGGGATCCAAAAAAATATGGTGGTTTTGATAGTCCAAC
GGTAGCTTATTCAGTCCTAGTGGTTGCTAAGGTGGAAAAAGGGAAATCGAAGAAGTTAA
AATCCGTTAAAGAGTTACTAGGGATCACAATTATGGAAAGAAGTTCCTTTGAAAAAAATC
CGATTGACTTTTTAGAAGCTAAAGGATATAAGGAAGTTAAAAAAGACTTAATCATTAAAC
TACCTAAATATAGTCTTTTTGAGTTAGAAAACGGTCGTAAACGGATGCTGGCTAGTGCCG
GAGAATTACAAAAAGGAAATGAGCTGGCTCTGCCAAGCAAATATGTGAATTTTTTATATT
TAGCTAGTCATTATGAAAAGTTGAAGGGTAGTCCAGAAGATAACGAACAAAAACAATTG
TTTGTGGAGCAGCATAAGCATTATTTAGATGAGATTATTGAGCAAATCAGTGAATTTTCT
AAGCGTGTTATTTTAGCAGATGCCAATTTAGATAAAGTTCTTAGTGCATATAACAAACAT
AGAGACAAACCAATACGTGAACAAGCAGAAAATATTATTCATTTATTTACGTTGACGAAT
CTTGGAGCTCCCGCTGCTTTTAAATATTTTGATACAACAATTGATCGTAAACGATATACGT
CTACAAAAGAAGTTTTAGATGCCACTCTTATCCATCAATCCATCACTGGTCTTTATGAAAC
ACGCATTGATTTGAGTCAGCTAGGAGGTGACTGA
Sequence for S. pyogenes Cas9_R70A (including N-terminal His-tag), recombinant
expressed in E.coli, purified and used for in vitro experiments; SEQ ID NO: 7:
ATGGGCCATCATCATCATCATCATCATCATCATCACAGCAGCGGCCATATCGAAGGTCGT
CATAGCGTCGACATGGATAAGAAATACTCAATAGGCTTAGATATCGGCACAAATAGCGTC
GGATGGGCGGTGATCACTGATGAATATAAGGTTCCGTCTAAAAAGTTCAAGGTTCTGGGA
AATACAGACCGCCACAGTATCAAAAAAAATCTTATAGGGGCTCTTTTATTTGACAGTGGA
GAGACAGCGGAAGCGACTCGTCTCAAACGGACAGCTCGTGCAAGGTATACACGTCGGAA
GAATCGTATTTGTTATCTACAGGAGATTTTTTCAAATGAGATGGCGAAAGTAGATGATAG
TTTCTTTCATCGACTTGAAGAGTCTTTTTTGGTGGAAGAAGACAAGAAGCATGAACGTCA
TCCTATTTTTGGAAATATAGTAGATGAAGTTGCTTATCATGAGAAATATCCAACTATCTAT
CATCTTCGAAAAAAATTGGTAGATTCTACTGATAAAGCGGATTTGCGCTTAATCTATTTGG
CCTTAGCGCATATGATTAAGTTTCGTGGTCATTTTTTGATTGAGGGAGATTTAAATCCTGA
TAATAGTGATGTGGACAAACTATTTATCCAGTTGGTACAAACCTACAATCAATTATTTGA
AGAAAACCCTATTAACGCAAGTGGAGTAGATGCTAAAGCGATTCTTTCTGCACGATTGAG
TAAATCAAGACGATTAGAAAATCTCATTGCTCAGCTCCCCGGTGAGAAGAAAAATGGCTT
ATTTGGGAATCTCATTGCTTTGTCATTGGGTTTGACCCCTAATTTTAAATCAAATTTTGATT
TGGCAGAAGATGCTAAATTACAGCTTTCAAAAGATACTTACGATGATGATTTAGATAATT
TATTGGCGCAAATTGGAGATCAATATGCTGATTTGTTTTTGGCAGCTAAGAATTTATCAGA
TGCTATTTTACTTTCAGATATCCTAAGAGTAAATACTGAAATAACTAAGGCTCCCCTATCA
GCTTCAATGATTAAACGCTACGATGAACATCATCAAGACTTGACTCTTTTAAAAGCTTTA
GTTCGACAACAACTTCCAGAAAAGTATAAAGAAATCTTTTTTGATCAATCAAAAAACGGA
TATGCAGGTTATATTGATGGGGGAGCTAGCCAAGAAGAATTTTATAAATTTATCAAACCA
ATTTTAGAAAAAATGGATGGTACTGAGGAATTATTGGTGAAACTAAATCGTGAAGATTTG
CTGCGCAAGCAACGGACCTTTGACAACGGCTCTATTCCCCATCAAATTCACTTGGGTGAG
CTGCATGCTATTTTGAGAAGACAAGAAGACTTTTATCCATTTTTAAAAGACAATCGTGAG
AAGATTGAAAAAATCTTGACTTTTCGAATTCCTTATTATGTTGGTCCATTGGCGCGTGGCA
ATAGTCGTTTTGCATGGATGACTCGGAAGTCTGAAGAAACAATTACCCCATGGAATTTTG
AAGAAGTTGTCGATAAAGGTGCTTCAGCTCAATCATTTATTGAACGCATGACAAACTTTG
ATAAAAATCTTCCAAATGAAAAAGTACTACCAAAACATAGTTTGCTTTATGAGTATTTTA
CGGTTTATAACGAATTGACAAAGGTCAAATATGTTACTGAAGGAATGCGAAAACCAGCA
TTTCTTTCAGGTGAACAGAAGAAAGCCATTGTTGATTTACTCTTCAAAACAAATCGAAAA
GTAACCGTTAAGCAATTAAAAGAAGATTATTTCAAAAAAATAGAATGTTTTGATAGTGTT
GAAATTTCAGGAGTTGAAGATAGATTTAATGCTTCATTAGGTACCTACCATGATTTGCTA
AAAATTATTAAAGATAAAGATTTTTTGGATAATGAAGAAAATGAAGATATCTTAGAGGAT
ATTGTTTTAACATTGACCTTATTTGAAGATAGGGAGATGATTGAGGAAAGACTTAAAACA
TATGCTCACCTCTTTGATGATAAGGTGATGAAACAGCTTAAACGTCGCCGTTATACTGGTT
GGGGACGTTTGTCTCGAAAATTGATTAATGGTATTAGGGATAAGCAATCTGGCAAAACAA
TATTAGATTTTTTGAAATCAGATGGTTTTGCCAATCGCAATTTTATGCAGCTGATCCATGA
TGATAGTTTGACATTTAAAGAAGACATTCAAAAAGCACAAGTGTCTGGACAAGGCGATA
GTTTACATGAACATATTGCAAATTTAGCTGGTAGCCCTGCTATTAAAAAAGGTATTTTACA
GACTGTAAAAGTTGTTGATGAATTGGTCAAAGTAATGGGGCGGCATAAGCCAGAAAATA
TCGTTATTGAAATGGCACGTGAAAATCAGACAACTCAAAAGGGCCAGAAAAATTCGCGA
GAGCGTATGAAACGAATCGAAGAAGGTATCAAAGAATTAGGAAGTCAGATTCTTAAAGA
GCATCCTGTTGAAAATACTCAATTGCAAAATGAAAAGCTCTATCTCTATTATCTCCAAAAT
GGAAGAGACATGTATGTGGACCAAGAATTAGATATTAATCGTTTAAGTGATTATGATGTC
GATCACATTGTTCCACAAAGTTTCCTTAAAGACGATTCAATAGACAATAAGGTCTTAACG
CGTTCTGATAAAAATCGTGGTAAATCGGATAACGTTCCAAGTGAAGAAGTAGTCAAAAA
GATGAAAAACTATTGGAGACAACTTCTAAACGCCAAGTTAATCACTCAACGTAAGTTTGA
TAATTTAACGAAAGCTGAACGTGGAGGTTTGAGTGAACTTGATAAAGCTGGTTTTATCAA
ACGCCAATTGGTTGAAACTCGCCAAATCACTAAGCATGTGGCACAAATTTTGGATAGTCG
CATGAATACTAAATACGATGAAAATGATAAACTTATTCGAGAGGTTAAAGTGATTACCTT
AAAATCTAAATTAGTTTCTGACTTCCGAAAAGATTTCCAATTCTATAAAGTACGTGAGATT
AACAATTACCATCATGCCCATGATGCGTATCTAAATGCCGTCGTTGGAACTGCTTTGATTA
AGAAATATCCAAAACTTGAATCGGAGTTTGTCTATGGTGATTATAAAGTTTATGATGTTC
GTAAAATGATTGCTAAGTCTGAGCAAGAAATAGGCAAAGCAACCGCAAAATATTTCTTTT
ACTCTAATATCATGAACTTCTTCAAAACAGAAATTACACTTGCAAATGGAGAGATTCGCA
AACGCCCTCTAATCGAAACTAATGGGGAAACTGGAGAAATTGTCTGGGATAAAGGGCGA
GATTTTGCCACAGTGCGCAAAGTATTGTCCATGCCCCAAGTCAATATTGTCAAGAAAACA
GAAGTACAGACAGGCGGATTCTCCAAGGAGTCAATTTTACCAAAAAGAAATTCGGACAA
GCTTATTGCTCGTAAAAAAGACTGGGATCCAAAAAAATATGGTGGTTTTGATAGTCCAAC
GGTAGCTTATTCAGTCCTAGTGGTTGCTAAGGTGGAAAAAGGGAAATCGAAGAAGTTAA
AATCCGTTAAAGAGTTACTAGGGATCACAATTATGGAAAGAAGTTCCTTTGAAAAAAATC
CGATTGACTTTTTAGAAGCTAAAGGATATAAGGAAGTTAAAAAAGACTTAATCATTAAAC
TACCTAAATATAGTCTTTTTGAGTTAGAAAACGGTCGTAAACGGATGCTGGCTAGTGCCG
GAGAATTACAAAAAGGAAATGAGCTGGCTCTGCCAAGCAAATATGTGAATTTTTTATATT
TAGCTAGTCATTATGAAAAGTTGAAGGGTAGTCCAGAAGATAACGAACAAAAACAATTG
TTTGTGGAGCAGCATAAGCATTATTTAGATGAGATTATTGAGCAAATCAGTGAATTTTCT
AAGCGTGTTATTTTAGCAGATGCCAATTTAGATAAAGTTCTTAGTGCATATAACAAACAT
AGAGACAAACCAATACGTGAACAAGCAGAAAATATTATTCATTTATTTACGTTGACGAAT
CTTGGAGCTCCCGCTGCTTTTAAATATTTTGATACAACAATTGATCGTAAACGATATACGT
CTACAAAAGAAGTTTTAGATGCCACTCTTATCCATCAATCCATCACTGGTCTTTATGAAAC
ACGCATTGATTTGAGTCAGCTAGGAGGTGACTGA
Sequence for S. pyogenes Cas9_R63A/Q768A (including N-terminal His-tag),
recombinant expressed in E. coli, purified and used for in vitro experiments;
SEQ ID NO: 8:
ATGGGCCATCATCATCATCATCATCATCATCATCACAGCAGCGGCCATATCGAAGGTCGT
CATAGCGTCGACATGGATAAGAAATACTCAATAGGCTTAGATATCGGCACAAATAGCGTC
GGATGGGCGGTGATCACTGATGAATATAAGGTTCCGTCTAAAAAGTTCAAGGTTCTGGGA
AATACAGACCGCCACAGTATCAAAAAAAATCTTATAGGGGCTCTTTTATTTGACAGTGGA
GAGACAGCGGAAGCGACTGCTCTCAAACGGACAGCTCGTAGAAGGTATACACGTCGGAA
GAATCGTATTTGTTATCTACAGGAGATTTTTTCAAATGAGATGGCGAAAGTAGATGATAG
TTTCTTTCATCGACTTGAAGAGTCTTTTTTGGTGGAAGAAGACAAGAAGCATGAACGTCA
TCCTATTTTTGGAAATATAGTAGATGAAGTTGCTTATCATGAGAAATATCCAACTATCTAT
CATCTTCGAAAAAAATTGGTAGATTCTACTGATAAAGCGGATTTGCGCTTAATCTATTTGG
CCTTAGCGCATATGATTAAGTTTCGTGGTCATTTTTTGATTGAGGGAGATTTAAATCCTGA
TAATAGTGATGTGGACAAACTATTTATCCAGTTGGTACAAACCTACAATCAATTATTTGA
AGAAAACCCTATTAACGCAAGTGGAGTAGATGCTAAAGCGATTCTTTCTGCACGATTGAG
TAAATCAAGACGATTAGAAAATCTCATTGCTCAGCTCCCCGGTGAGAAGAAAAATGGCTT
ATTTGGGAATCTCATTGCTTTGTCATTGGGTTTGACCCCTAATTTTAAATCAAATTTTGATT
TGGCAGAAGATGCTAAATTACAGCTTTCAAAAGATACTTACGATGATGATTTAGATAATT
TATTGGCGCAAATTGGAGATCAATATGCTGATTTGTTTTTGGCAGCTAAGAATTTATCAGA
TGCTATTTTACTTTCAGATATCCTAAGAGTAAATACTGAAATAACTAAGGCTCCCCTATCA
GCTTCAATGATTAAACGCTACGATGAACATCATCAAGACTTGACTCTTTTAAAAGCTTTA
GTTCGACAACAACTTCCAGAAAAGTATAAAGAAATCTTTTTTGATCAATCAAAAAACGGA
TATGCAGGTTATATTGATGGGGGAGCTAGCCAAGAAGAATTTTATAAATTTATCAAACCA
ATTTTAGAAAAAATGGATGGTACTGAGGAATTATTGGTGAAACTAAATCGTGAAGATTTG
CTGCGCAAGCAACGGACCTTTGACAACGGCTCTATTCCCCATCAAATTCACTTGGGTGAG
CTGCATGCTATTTTGAGAAGACAAGAAGACTTTTATCCATTTTTAAAAGACAATCGTGAG
AAGATTGAAAAAATCTTGACTTTTCGAATTCCTTATTATGTTGGTCCATTGGCGCGTGGCA
ATAGTCGTTTTGCATGGATGACTCGGAAGTCTGAAGAAACAATTACCCCATGGAATTTTG
AAGAAGTTGTCGATAAAGGTGCTTCAGCTCAATCATTTATTGAACGCATGACAAACTTTG
ATAAAAATCTTCCAAATGAAAAAGTACTACCAAAACATAGTTTGCTTTATGAGTATTTTA
CGGTTTATAACGAATTGACAAAGGTCAAATATGTTACTGAAGGAATGCGAAAACCAGCA
TTTCTTTCAGGTGAACAGAAGAAAGCCATTGTTGATTTACTCTTCAAAACAAATCGAAAA
GTAACCGTTAAGCAATTAAAAGAAGATTATTTCAAAAAAATAGAATGTTTTGATAGTGTT
GAAATTTCAGGAGTTGAAGATAGATTTAATGCTTCATTAGGTACCTACCATGATTTGCTA
AAAATTATTAAAGATAAAGATTTTTTGGATAATGAAGAAAATGAAGATATCTTAGAGGAT
ATTGTTTTAACATTGACCTTATTTGAAGATAGGGAGATGATTGAGGAAAGACTTAAAACA
TATGCTCACCTCTTTGATGATAAGGTGATGAAACAGCTTAAACGTCGCCGTTATACTGGTT
GGGGACGTTTGTCTCGAAAATTGATTAATGGTATTAGGGATAAGCAATCTGGCAAAACAA
TATTAGATTTTTTGAAATCAGATGGTTTTGCCAATCGCAATTTTATGCAGCTGATCCATGA
TGATAGTTTGACATTTAAAGAAGACATTCAAAAAGCACAAGTGTCTGGACAAGGCGATA
GTTTACATGAACATATTGCAAATTTAGCTGGTAGCCCTGCTATTAAAAAAGGTATTTTACA
GACTGTAAAAGTTGTTGATGAATTGGTCAAAGTAATGGGGCGGCATAAGCCAGAAAATA
TCGTTATTGAAATGGCACGTGAAAATGCGACAACTCAAAAGGGCCAGAAAAATTCGCGA
GAGCGTATGAAACGAATCGAAGAAGGTATCAAAGAATTAGGAAGTCAGATTCTTAAAGA
GCATCCTGTTGAAAATACTCAATTGCAAAATGAAAAGCTCTATCTCTATTATCTCCAAAAT
GGAAGAGACATGTATGTGGACCAAGAATTAGATATTAATCGTTTAAGTGATTATGATGTC
GATCACATTGTTCCACAAAGTTTCCTTAAAGACGATTCAATAGACAATAAGGTCTTAACG
CGTTCTGATAAAAATCGTGGTAAATCGGATAACGTTCCAAGTGAAGAAGTAGTCAAAAA
GATGAAAAACTATTGGAGACAACTTCTAAACGCCAAGTTAATCACTCAACGTAAGTTTGA
TAATTTAACGAAAGCTGAACGTGGAGGTTTGAGTGAACTTGATAAAGCTGGTTTTATCAA
ACGCCAATTGGTTGAAACTCGCCAAATCACTAAGCATGTGGCACAAATTTTGGATAGTCG
CATGAATACTAAATACGATGAAAATGATAAACTTATTCGAGAGGTTAAAGTGATTACCTT
AAAATCTAAATTAGTTTCTGACTTCCGAAAAGATTTCCAATTCTATAAAGTACGTGAGATT
AACAATTACCATCATGCCCATGATGCGTATCTAAATGCCGTCGTTGGAACTGCTTTGATTA
AGAAATATCCAAAACTTGAATCGGAGTTTGTCTATGGTGATTATAAAGTTTATGATGTTC
GTAAAATGATTGCTAAGTCTGAGCAAGAAATAGGCAAAGCAACCGCAAAATATTTCTTTT
ACTCTAATATCATGAACTTCTTCAAAACAGAAATTACACTTGCAAATGGAGAGATTCGCA
AACGCCCTCTAATCGAAACTAATGGGGAAACTGGAGAAATTGTCTGGGATAAAGGGCGA
GATTTTGCCACAGTGCGCAAAGTATTGTCCATGCCCCAAGTCAATATTGTCAAGAAAACA
GAAGTACAGACAGGCGGATTCTCCAAGGAGTCAATTTTACCAAAAAGAAATTCGGACAA
GCTTATTGCTCGTAAAAAAGACTGGGATCCAAAAAAATATGGTGGTTTTGATAGTCCAAC
GGTAGCTTATTCAGTCCTAGTGGTTGCTAAGGTGGAAAAAGGGAAATCGAAGAAGTTAA
AATCCGTTAAAGAGTTACTAGGGATCACAATTATGGAAAGAAGTTCCTTTGAAAAAAATC
CGATTGACTTTTTAGAAGCTAAAGGATATAAGGAAGTTAAAAAAGACTTAATCATTAAAC
TACCTAAATATAGTCTTTTTGAGTTAGAAAACGGTCGTAAACGGATGCTGGCTAGTGCCG
GAGAATTACAAAAAGGAAATGAGCTGGCTCTGCCAAGCAAATATGTGAATTTTTTATATT
TAGCTAGTCATTATGAAAAGTTGAAGGGTAGTCCAGAAGATAACGAACAAAAACAATTG
TTTGTGGAGCAGCATAAGCATTATTTAGATGAGATTATTGAGCAAATCAGTGAATTTTCT
AAGCGTGTTATTTTAGCAGATGCCAATTTAGATAAAGTTCTTAGTGCATATAACAAACAT
AGAGACAAACCAATACGTGAACAAGCAGAAAATATTATTCATTTATTTACGTTGACGAAT
CTTGGAGCTCCCGCTGCTTTTAAATATTTTGATACAACAATTGATCGTAAACGATATACGT
CTACAAAAGAAGTTTTAGATGCCACTCTTATCCATCAATCCATCACTGGTCTTTATGAAAC
ACGCATTGATTTGAGTCAGCTAGGAGGTGACTGA
Sequence for S. pyogenes Cas9 Q768A (including N-terminal His-tag), recombinant
expressed in E.coli, purified and used for in vitro experiments; SEQ ID NO: 9:
ATGGGCCATCATCATCATCATCATCATCATCATCACAGCAGCGGCCATATCGAAGGTCGT
CATAGCGTCGACATGGATAAGAAATACTCAATAGGCTTAGATATCGGCACAAATAGCGTC
GGATGGGCGGTGATCACTGATGAATATAAGGTTCCGTCTAAAAAGTTCAAGGTTCTGGGA
AATACAGACCGCCACAGTATCAAAAAAAATCTTATAGGGGCTCTTTTATTTGACAGTGGA
GAGACAGCGGAAGCGACTCGTCTCAAACGGACAGCTCGTAGAAGGTATACACGTCGGAA
GAATCGTATTTGTTATCTACAGGAGATTTTTTCAAATGAGATGGCGAAAGTAGATGATAG
TTTCTTTCATCGACTTGAAGAGTCTTTTTTGGTGGAAGAAGACAAGAAGCATGAACGTCA
TCCTATTTTTGGAAATATAGTAGATGAAGTTGCTTATCATGAGAAATATCCAACTATCTAT
CATCTGCGAAAAAAATTGGTAGATTCTACTGATAAAGCGGATTTGCGCTTAATCTATTTG
GCCTTAGCGCATATGATTAAGTTTCGTGGTCATTTTTTGATTGAGGGAGATTTAAATCCTG
ATAATAGTGATGTGGACAAACTATTTATCCAGTTGGTACAAACCTACAATCAATTATTTG
AAGAAAACCCTATTAACGCAAGTGGAGTAGATGCTAAAGCGATTCTTTCTGCACGATTGA
GTAAATCAAGACGATTAGAAAATCTCATTGCTCAGCTCCCCGGTGAGAAGAAAAATGGCT
TATTTGGGAATCTCATTGCTTTGTCATTGGGTTTGACCCCTAATTTTAAATCAAATTTTGAT
TTGGCAGAAGATGCTAAATTACAGCTTTCAAAAGATACTTACGATGATGATTTAGATAAT
TTATTGGCGCAAATTGGAGATCAATATGCTGATTTGTTTTTGGCAGCTAAGAATTTATCAG
ATGCTATTTTACTTTCAGATATCCTAAGAGTAAATACTGAAATAACTAAGGCTCCCCTATC
AGCTTCAATGATTAAACGCTACGATGAACATCATCAAGACTTGACTCTTTTAAAAGCTTT
AGTTCGACAACAACTTCCAGAAAAGTATAAAGAAATCTTTTTTGATCAATCAAAAAACGG
ATATGCAGGTTATATTGATGGGGGAGCTAGCCAAGAAGAATTTTATAAATTTATCAAACC
AATTTTAGAAAAAATGGATGGTACTGAGGAATTATTGGTGAAACTAAATCGTGAAGATTT
GCTGCGCAAGCAACGGACCTTTGACAACGGCTCTATTCCCCATCAAATTCACTTGGGTGA
GCTGCATGCTATTTTGAGAAGACAAGAAGACTTTTATCCATTTTTAAAAGACAATCGTGA
GAAGATTGAAAAAATCTTGACTTTTCGAATTCCTTATTATGTTGGTCCATTGGCGCGTGGC
AATAGTCGTTTTGCATGGATGACTCGGAAGTCTGAAGAAACAATTACCCCATGGAATTTT
GAAGAAGTTGTCGATAAAGGTGCTTCAGCTCAATCATTTATTGAACGCATGACAAACTTT
GATAAAAATCTTCCAAATGAAAAAGTACTACCAAAACATAGTTTGCTTTATGAGTATTTT
ACGGTTTATAACGAATTGACAAAGGTCAAATATGTTACTGAAGGAATGCGAAAACCAGC
ATTTCTTTCAGGTGAACAGAAGAAAGCCATTGTTGATTTACTCTTCAAAACAAATCGAAA
AGTAACCGTTAAGCAATTAAAAGAAGATTATTTCAAAAAAATAGAATGTTTTGATAGTGT
TGAAATTTCAGGAGTTGAAGATAGATTTAATGCTTCATTAGGTACCTACCATGATTTGCTA
AAAATTATTAAAGATAAAGATTTTTTGGATAATGAAGAAAATGAAGATATCTTAGAGGAT
ATTGTTTTAACATTGACCTTATTTGAAGATAGGGAGATGATTGAGGAAAGACTTAAAACA
TATGCTCACCTCTTTGATGATAAGGTGATGAAACAGCTTAAACGTCGCCGTTATACTGGTT
GGGGACGTTTGTCTCGAAAATTGATTAATGGTATTAGGGATAAGCAATCTGGCAAAACAA
TATTAGATTTTTTGAAATCAGATGGTTTTGCCAATCGCAATTTTATGCAGCTGATCCATGA
TGATAGTTTGACATTTAAAGAAGACATTCAAAAAGCACAAGTGTCTGGACAAGGCGATA
GTTTACATGAACATATTGCAAATTTAGCTGGTAGCCCTGCTATTAAAAAAGGTATTTTACA
GACTGTAAAAGTTGTTGATGAATTGGTCAAAGTAATGGGGCGGCATAAGCCAGAAAATA
TCGTTATTGAAATGGCACGTGAAAATGCGACAACTCAAAAGGGCCAGAAAAATTCGCGA
GAGCGTATGAAACGAATCGAAGAAGGTATCAAAGAATTAGGAAGTCAGATTCTTAAAGA
GCATCCTGTTGAAAATACTCAATTGCAAAATGAAAAGCTCTATCTCTATTATCTCCAAAAT
GGAAGAGACATGTATGTGGACCAAGAATTAGATATTAATCGTTTAAGTGATTATGATGTC
GATCACATTGTTCCACAAAGTTTCCTTAAAGACGATTCAATAGACAATAAGGTCTTAACG
CGTTCTGATAAAAATCGTGGTAAATCGGATAACGTTCCAAGTGAAGAAGTAGTCAAAAA
GATGAAAAACTATTGGAGACAACTTCTAAACGCCAAGTTAATCACTCAACGTAAGTTTGA
TAATTTAACGAAAGCTGAACGTGGAGGTTTGAGTGAACTTGATAAAGCTGGTTTTATCAA
ACGCCAATTGGTTGAAACTCGCCAAATCACTAAGCATGTGGCACAAATTTTGGATAGTCG
CATGAATACTAAATACGATGAAAATGATAAACTTATTCGAGAGGTTAAAGTGATTACCTT
AAAATCTAAATTAGTTTCTGACTTCCGAAAAGATTTCCAATTCTATAAAGTACGTGAGATT
AACAATTACCATCATGCCCATGATGCGTATCTAAATGCCGTCGTTGGAACTGCTTTGATTA
AGAAATATCCAAAACTTGAATCGGAGTTTGTCTATGGTGATTATAAAGTTTATGATGTTC
GTAAAATGATTGCTAAGTCTGAGCAAGAAATAGGCAAAGCAACCGCAAAATATTTCTTTT
ACTCTAATATCATGAACTTCTTCAAAACAGAAATTACACTTGCAAATGGAGAGATTCGCA
AACGCCCTCTAATCGAAACTAATGGGGAAACTGGAGAAATTGTCTGGGATAAAGGGCGA
GATTTTGCCACAGTGCGCAAAGTATTGTCCATGCCCCAAGTCAATATTGTCAAGAAAACA
GAAGTACAGACAGGCGGATTCTCCAAGGAGTCAATTTTACCAAAAAGAAATTCGGACAA
GCTTATTGCTCGTAAAAAAGACTGGGATCCAAAAAAATATGGTGGTTTTGATAGTCCAAC
GGTAGCTTATTCAGTCCTAGTGGTTGCTAAGGTGGAAAAAGGGAAATCGAAGAAGTTAA
AATCCGTTAAAGAGTTACTAGGGATCACAATTATGGAAAGAAGTTCCTTTGAAAAAAATC
CGATTGACTTTTTAGAAGCTAAAGGATATAAGGAAGTTAAAAAAGACTTAATCATTAAAC
TACCTAAATATAGTCTTTTTGAGTTAGAAAACGGTCGTAAACGGATGCTGGCTAGTGCCG
GAGAATTACAAAAAGGAAATGAGCTGGCTCTGCCAAGCAAATATGTGAATTTTTTATATT
TAGCTAGTCATTATGAAAAGTTGAAGGGTAGTCCAGAAGATAACGAACAAAAACAATTG
TTTGTGGAGCAGCATAAGCATTATTTAGATGAGATTATTGAGCAAATCAGTGAATTTTCT
AAGCGTGTTATTTTAGCAGATGCCAATTTAGATAAAGTTCTTAGTGCATATAACAAACAT
AGAGACAAACCAATACGTGAACAAGCAGAAAATATTATTCATTTATTTACGTTGACGAAT
CTTGGAGCTCCCGCTGCTTTTAAATATTTTGATACAACAATTGATCGTAAACGATATACGT
CTACAAAAGAAGTTTTAGATGCCACTCTTATCCATCAATCCATCACTGGTCTTTATGAAAC
ACGCATTGATTTGAGTCAGCTAGGAGGTGACTGA
Sequence for S. pyogenes Cas9_R66A/Q768A (including N-terminal His-tag),
recombinant expressed in E.coli, purified and used for in vitro experiments;
SEQ ID NO: 10:
ATGGGCCATCATCATCATCATCATCATCATCATCACAGCAGCGGCCATATCGAAGGTCGT
CATAGCGTCGACATGGATAAGAAATACTCAATAGGCTTAGATATCGGCACAAATAGCGTC
GGATGGGCGGTGATCACTGATGAATATAAGGTTCCGTCTAAAAAGTTCAAGGTTCTGGGA
AATACAGACCGCCACAGTATCAAAAAAAATCTTATAGGGGCTCTTTTATTTGACAGTGGA
GAGACAGCGGAAGCGACTCGTCTCAAAGCGACAGCTCGTAGAAGGTATACACGTCGGAA
GAATCGTATTTGTTATCTACAGGAGATTTTTTCAAATGAGATGGCGAAAGTAGATGATAG
TTTCTTTCATCGACTTGAAGAGTCTTTTTTGGTGGAAGAAGACAAGAAGCATGAACGTCA
TCCTATTTTTGGAAATATAGTAGATGAAGTTGCTTATCATGAGAAATATCCAACTATCTAT
CATCTTCGAAAAAAATTGGTAGATTCTACTGATAAAGCGGATTTGCGCTTAATCTATTTGG
CCTTAGCGCATATGATTAAGTTTCGTGGTCATTTTTTGATTGAGGGAGATTTAAATCCTGA
TAATAGTGATGTGGACAAACTATTTATCCAGTTGGTACAAACCTACAATCAATTATTTGA
AGAAAACCCTATTAACGCAAGTGGAGTAGATGCTAAAGCGATTCTTTCTGCACGATTGAG
TAAATCAAGACGATTAGAAAATCTCATTGCTCAGCTCCCCGGTGAGAAGAAAAATGGCTT
ATTTGGGAATCTCATTGCTTTGTCATTGGGTTTGACCCCTAATTTTAAATCAAATTTTGATT
TGGCAGAAGATGCTAAATTACAGCTTTCAAAAGATACTTACGATGATGATTTAGATAATT
TATTGGCGCAAATTGGAGATCAATATGCTGATTTGTTTTTGGCAGCTAAGAATTTATCAGA
TGCTATTTTACTTTCAGATATCCTAAGAGTAAATACTGAAATAACTAAGGCTCCCCTATCA
GCTTCAATGATTAAACGCTACGATGAACATCATCAAGACTTGACTCTTTTAAAAGCTTTA
GTTCGACAACAACTTCCAGAAAAGTATAAAGAAATCTTTTTTGATCAATCAAAAAACGGA
TATGCAGGTTATATTGATGGGGGAGCTAGCCAAGAAGAATTTTATAAATTTATCAAACCA
ATTTTAGAAAAAATGGATGGTACTGAGGAATTATTGGTGAAACTAAATCGTGAAGATTTG
CTGCGCAAGCAACGGACCTTTGACAACGGCTCTATTCCCCATCAAATTCACTTGGGTGAG
CTGCATGCTATTTTGAGAAGACAAGAAGACTTTTATCCATTTTTAAAAGACAATCGTGAG
AAGATTGAAAAAATCTTGACTTTTCGAATTCCTTATTATGTTGGTCCATTGGCGCGTGGCA
ATAGTCGTTTTGCATGGATGACTCGGAAGTCTGAAGAAACAATTACCCCATGGAATTTTG
AAGAAGTTGTCGATAAAGGTGCTTCAGCTCAATCATTTATTGAACGCATGACAAACTTTG
ATAAAAATCTTCCAAATGAAAAAGTACTACCAAAACATAGTTTGCTTTATGAGTATTTTA
CGGTTTATAACGAATTGACAAAGGTCAAATATGTTACTGAAGGAATGCGAAAACCAGCA
TTTCTTTCAGGTGAACAGAAGAAAGCCATTGTTGATTTACTCTTCAAAACAAATCGAAAA
GTAACCGTTAAGCAATTAAAAGAAGATTATTTCAAAAAAATAGAATGTTTTGATAGTGTT
GAAATTTCAGGAGTTGAAGATAGATTTAATGCTTCATTAGGTACCTACCATGATTTGCTA
AAAATTATTAAAGATAAAGATTTTTTGGATAATGAAGAAAATGAAGATATCTTAGAGGAT
ATTGTTTTAACATTGACCTTATTTGAAGATAGGGAGATGATTGAGGAAAGACTTAAAACA
TATGCTCACCTCTTTGATGATAAGGTGATGAAACAGCTTAAACGTCGCCGTTATACTGGTT
GGGGACGTTTGTCTCGAAAATTGATTAATGGTATTAGGGATAAGCAATCTGGCAAAACAA
TATTAGATTTTTTGAAATCAGATGGTTTTGCCAATCGCAATTTTATGCAGCTGATCCATGA
TGATAGTTTGACATTTAAAGAAGACATTCAAAAAGCACAAGTGTCTGGACAAGGCGATA
GTTTACATGAACATATTGCAAATTTAGCTGGTAGCCCTGCTATTAAAAAAGGTATTTTACA
GACTGTAAAAGTTGTTGATGAATTGGTCAAAGTAATGGGGCGGCATAAGCCAGAAAATA
TCGTTATTGAAATGGCACGTGAAAATGCGACAACTCAAAAGGGCCAGAAAAATTCGCGA
GAGCGTATGAAACGAATCGAAGAAGGTATCAAAGAATTAGGAAGTCAGATTCTTAAAGA
GCATCCTGTTGAAAATACTCAATTGCAAAATGAAAAGCTCTATCTCTATTATCTCCAAAAT
GGAAGAGACATGTATGTGGACCAAGAATTAGATATTAATCGTTTAAGTGATTATGATGTC
GATCACATTGTTCCACAAAGTTTCCTTAAAGACGATTCAATAGACAATAAGGTCTTAACG
CGTTCTGATAAAAATCGTGGTAAATCGGATAACGTTCCAAGTGAAGAAGTAGTCAAAAA
GATGAAAAACTATTGGAGACAACTTCTAAACGCCAAGTTAATCACTCAACGTAAGTTTGA
TAATTTAACGAAAGCTGAACGTGGAGGTTTGAGTGAACTTGATAAAGCTGGTTTTATCAA
ACGCCAATTGGTTGAAACTCGCCAAATCACTAAGCATGTGGCACAAATTTTGGATAGTCG
CATGAATACTAAATACGATGAAAATGATAAACTTATTCGAGAGGTTAAAGTGATTACCTT
AAAATCTAAATTAGTTTCTGACTTCCGAAAAGATTTCCAATTCTATAAAGTACGTGAGATT
AACAATTACCATCATGCCCATGATGCGTATCTAAATGCCGTCGTTGGAACTGCTTTGATTA
AGAAATATCCAAAACTTGAATCGGAGTTTGTCTATGGTGATTATAAAGTTTATGATGTTC
GTAAAATGATTGCTAAGTCTGAGCAAGAAATAGGCAAAGCAACCGCAAAATATTTCTTTT
ACTCTAATATCATGAACTTCTTCAAAACAGAAATTACACTTGCAAATGGAGAGATTCGCA
AACGCCCTCTAATCGAAACTAATGGGGAAACTGGAGAAATTGTCTGGGATAAAGGGCGA
GATTTTGCCACAGTGCGCAAAGTATTGTCCATGCCCCAAGTCAATATTGTCAAGAAAACA
GAAGTACAGACAGGCGGATTCTCCAAGGAGTCAATTTTACCAAAAAGAAATTCGGACAA
GCTTATTGCTCGTAAAAAAGACTGGGATCCAAAAAAATATGGTGGTTTTGATAGTCCAAC
GGTAGCTTATTCAGTCCTAGTGGTTGCTAAGGTGGAAAAAGGGAAATCGAAGAAGTTAA
AATCCGTTAAAGAGTTACTAGGGATCACAATTATGGAAAGAAGTTCCTTTGAAAAAAATC
CGATTGACTTTTTAGAAGCTAAAGGATATAAGGAAGTTAAAAAAGACTTAATCATTAAAC
TACCTAAATATAGTCTTTTTGAGTTAGAAAACGGTCGTAAACGGATGCTGGCTAGTGCCG
GAGAATTACAAAAAGGAAATGAGCTGGCTCTGCCAAGCAAATATGTGAATTTTTTATATT
TAGCTAGTCATTATGAAAAGTTGAAGGGTAGTCCAGAAGATAACGAACAAAAACAATTG
TTTGTGGAGCAGCATAAGCATTATTTAGATGAGATTATTGAGCAAATCAGTGAATTTTCT
AAGCGTGTTATTTTAGCAGATGCCAATTTAGATAAAGTTCTTAGTGCATATAACAAACAT
AGAGACAAACCAATACGTGAACAAGCAGAAAATATTATTCATTTATTTACGTTGACGAAT
CTTGGAGCTCCCGCTGCTTTTAAATATTTTGATACAACAATTGATCGTAAACGATATACGT
CTACAAAAGAAGTTTTAGATGCCACTCTTATCCATCAATCCATCACTGGTCTTTATGAAAC
ACGCATTGATTTGAGTCAGCTAGGAGGTGACTGA
Sequence for S. pyogenes Cas9 (including N-terminal His-tag), used for in
vivo experiments in E. coli, note that only the coding sequence for the His-tag
differs, the CDS for Cas9 is the same as for the recombinant purified ones;
SEQ ID NO: 11:
atgaaacatcaccatcaccatcacaacactagtCATATCGAAGGTCGTCATAGCGTCGACATGGATAAGAAA
TACTCAATAGGCTTAGATATCGGCACAAATAGCGTCGGATGGGCGGTGATCACTGATGAA
TATAAGGTTCCGTCTAAAAAGTTCAAGGTTCTGGGAAATACAGACCGCCACAGTATCAAA
AAAAATCTTATAGGGGCTCTTTTATTTGACAGTGGAGAGACAGCGGAAGCGACTCGTCTC
AAACGGACAGCTCGTAGAAGGTATACACGTCGGAAGAATCGTATTTGTTATCTACAGGAG
ATTTTTTCAAATGAGATGGCGAAAGTAGATGATAGTTTCTTTCATCGACTTGAAGAGTCTT
TTTTGGTGGAAGAAGACAAGAAGCATGAACGTCATCCTATTTTTGGAAATATAGTAGATG
AAGTTGCTTATCATGAGAAATATCCAACTATCTATCATCTGCGAAAAAAATTGGTAGATT
CTACTGATAAAGCGGATTTGCGCTTAATCTATTTGGCCTTAGCGCATATGATTAAGTTTCG
TGGTCATTTTTTGATTGAGGGAGATTTAAATCCTGATAATAGTGATGTGGACAAACTATTT
ATCCAGTTGGTACAAACCTACAATCAATTATTTGAAGAAAACCCTATTAACGCAAGTGGA
GTAGATGCTAAAGCGATTCTTTCTGCACGATTGAGTAAATCAAGACGATTAGAAAATCTC
ATTGCTCAGCTCCCCGGTGAGAAGAAAAATGGCTTATTTGGGAATCTCATTGCTTTGTCAT
TGGGTTTGACCCCTAATTTTAAATCAAATTTTGATTTGGCAGAAGATGCTAAATTACAGCT
TTCAAAAGATACTTACGATGATGATTTAGATAATTTATTGGCGCAAATTGGAGATCAATA
TGCTGATTTGTTTTTGGCAGCTAAGAATTTATCAGATGCTATTTTACTTTCAGATATCCTA
AGAGTAAATACTGAAATAACTAAGGCTCCCCTATCAGCTTCAATGATTAAACGCTACGAT
GAACATCATCAAGACTTGACTCTTTTAAAAGCTTTAGTTCGACAACAACTTCCAGAAAAG
TATAAAGAAATCTTTTTTGATCAATCAAAAAACGGATATGCAGGTTATATTGATGGGGGA
GCTAGCCAAGAAGAATTTTATAAATTTATCAAACCAATTTTAGAAAAAATGGATGGTACT
GAGGAATTATTGGTGAAACTAAATCGTGAAGATTTGCTGCGCAAGCAACGGACCTTTGAC
AACGGCTCTATTCCCCATCAAATTCACTTGGGTGAGCTGCATGCTATTTTGAGAAGACAA
GAAGACTTTTATCCATTTTTAAAAGACAATCGTGAGAAGATTGAAAAAATCTTGACTTTT
CGAATTCCTTATTATGTTGGTCCATTGGCGCGTGGCAATAGTCGTTTTGCATGGATGACTC
GGAAGTCTGAAGAAACAATTACCCCATGGAATTTTGAAGAAGTTGTCGATAAAGGTGCTT
CAGCTCAATCATTTATTGAACGCATGACAAACTTTGATAAAAATCTTCCAAATGAAAAAG
TACTACCAAAACATAGTTTGCTTTATGAGTATTTTACGGTTTATAACGAATTGACAAAGGT
CAAATATGTTACTGAAGGAATGCGAAAACCAGCATTTCTTTCAGGTGAACAGAAGAAAG
CCATTGTTGATTTACTCTTCAAAACAAATCGAAAAGTAACCGTTAAGCAATTAAAAGAAG
ATTATTTCAAAAAAATAGAATGTTTTGATAGTGTTGAAATTTCAGGAGTTGAAGATAGAT
TTAATGCTTCATTAGGTACCTACCATGATTTGCTAAAAATTATTAAAGATAAAGATTTTTT
GGATAATGAAGAAAATGAAGATATCTTAGAGGATATTGTTTTAACATTGACCTTATTTGA
AGATAGGGAGATGATTGAGGAAAGACTTAAAACATATGCTCACCTCTTTGATGATAAGGT
GATGAAACAGCTTAAACGTCGCCGTTATACTGGTTGGGGACGTTTGTCTCGAAAATTGAT
TAATGGTATTAGGGATAAGCAATCTGGCAAAACAATATTAGATTTTTTGAAATCAGATGG
TTTTGCCAATCGCAATTTTATGCAGCTGATCCATGATGATAGTTTGACATTTAAAGAAGAC
ATTCAAAAAGCACAAGTGTCTGGACAAGGCGATAGTTTACATGAACATATTGCAAATTTA
GCTGGTAGCCCTGCTATTAAAAAAGGTATTTTACAGACTGTAAAAGTTGTTGATGAATTG
GTCAAAGTAATGGGGCGGCATAAGCCAGAAAATATCGTTATTGAAATGGCACGTGAAAA
TCAGACAACTCAAAAGGGCCAGAAAAATTCGCGAGAGCGTATGAAACGAATCGAAGAAG
GTATCAAAGAATTAGGAAGTCAGATTCTTAAAGAGCATCCTGTTGAAAATACTCAATTGC
AAAATGAAAAGCTCTATCTCTATTATCTCCAAAATGGAAGAGACATGTATGTGGACCAAG
AATTAGATATTAATCGTTTAAGTGATTATGATGTCGATCACATTGTTCCACAAAGTTTCCT
TAAAGACGATTCAATAGACAATAAGGTCTTAACGCGTTCTGATAAAAATCGTGGTAAATC
GGATAACGTTCCAAGTGAAGAAGTAGTCAAAAAGATGAAAAACTATTGGAGACAACTTC
TAAACGCCAAGTTAATCACTCAACGTAAGTTTGATAATTTAACGAAAGCTGAACGTGGAG
GTTTGAGTGAACTTGATAAAGCTGGTTTTATCAAACGCCAATTGGTTGAAACTCGCCAAA
TCACTAAGCATGTGGCACAAATTTTGGATAGTCGCATGAATACTAAATACGATGAAAATG
ATAAACTTATTCGAGAGGTTAAAGTGATTACCTTAAAATCTAAATTAGTTTCTGACTTCCG
AAAAGATTTCCAATTCTATAAAGTACGTGAGATTAACAATTACCATCATGCCCATGATGC
GTATCTAAATGCCGTCGTTGGAACTGCTTTGATTAAGAAATATCCAAAACTTGAATCGGA
GTTTGTCTATGGTGATTATAAAGTTTATGATGTTCGTAAAATGATTGCTAAGTCTGAGCAA
GAAATAGGCAAAGCAACCGCAAAATATTTCTTTTACTCTAATATCATGAACTTCTTCAAA
ACAGAAATTACACTTGCAAATGGAGAGATTCGCAAACGCCCTCTAATCGAAACTAATGG
GGAAACTGGAGAAATTGTCTGGGATAAAGGGCGAGATTTTGCCACAGTGCGCAAAGTAT
TGTCCATGCCCCAAGTCAATATTGTCAAGAAAACAGAAGTACAGACAGGCGGATTCTCCA
AGGAGTCAATTTTACCAAAAAGAAATTCGGACAAGCTTATTGCTCGTAAAAAAGACTGG
GATCCAAAAAAATATGGTGGTTTTGATAGTCCAACGGTAGCTTATTCAGTCCTAGTGGTT
GCTAAGGTGGAAAAAGGGAAATCGAAGAAGTTAAAATCCGTTAAAGAGTTACTAGGGAT
CACAATTATGGAAAGAAGTTCCTTTGAAAAAAATCCGATTGACTTTTTAGAAGCTAAAGG
ATATAAGGAAGTTAAAAAAGACTTAATCATTAAACTACCTAAATATAGTCTTTTTGAGTT
AGAAAACGGTCGTAAACGGATGCTGGCTAGTGCCGGAGAATTACAAAAAGGAAATGAGC
TGGCTCTGCCAAGCAAATATGTGAATTTTTTATATTTAGCTAGTCATTATGAAAAGTTGAA
GGGTAGTCCAGAAGATAACGAACAAAAACAATTGTTTGTGGAGCAGCATAAGCATTATTT
AGATGAGATTATTGAGCAAATCAGTGAATTTTCTAAGCGTGTTATTTTAGCAGATGCCAA
TTTAGATAAAGTTCTTAGTGCATATAACAAACATAGAGACAAACCAATACGTGAACAAGC
AGAAAATATTATTCATTTATTTACGTTGACGAATCTTGGAGCTCCCGCTGCTTTTAAATAT
TTTGATACAACAATTGATCGTAAACGATATACGTCTACAAAAGAAGTTTTAGATGCCACT
CTTATCCATCAATCCATCACTGGTCTTTATGAAACACGCATTGATTTGAGTCAGCTAGGAG
GTGACTGA
Sequence for S. pyogenes Cas9_R63A (including N-terminal His-tag), used for in
vivo experiments in E. coli, note that only the coding sequence for the His-tag
differs, the CDS for Cas9 is the same as for the recombinant purified ones;
SEQ ID NO: 12:
atgaaacatcaccatcaccatcacaacactagtCATATCGAAGGTCGTCATAGCGTCGACATGGATAAGAAA
TACTCAATAGGCTTAGATATCGGCACAAATAGCGTCGGATGGGCGGTGATCACTGATGAA
TATAAGGTTCCGTCTAAAAAGTTCAAGGTTCTGGGAAATACAGACCGCCACAGTATCAAA
AAAAATCTTATAGGGGCTCTTTTATTTGACAGTGGAGAGACAGCGGAAGCGACTGCTCTC
AAACGGACAGCTCGTAGAAGGTATACACGTCGGAAGAATCGTATTTGTTATCTACAGGAG
ATTTTTTCAAATGAGATGGCGAAAGTAGATGATAGTTTCTTTCATCGACTTGAAGAGTCTT
TTTTGGTGGAAGAAGACAAGAAGCATGAACGTCATCCTATTTTTGGAAATATAGTAGATG
AAGTTGCTTATCATGAGAAATATCCAACTATCTATCATCTTCGAAAAAAATTGGTAGATT
CTACTGATAAAGCGGATTTGCGCTTAATCTATTTGGCCTTAGCGCATATGATTAAGTTTCG
TGGTCATTTTTTGATTGAGGGAGATTTAAATCCTGATAATAGTGATGTGGACAAACTATTT
ATCCAGTTGGTACAAACCTACAATCAATTATTTGAAGAAAACCCTATTAACGCAAGTGGA
GTAGATGCTAAAGCGATTCTTTCTGCACGATTGAGTAAATCAAGACGATTAGAAAATCTC
ATTGCTCAGCTCCCCGGTGAGAAGAAAAATGGCTTATTTGGGAATCTCATTGCTTTGTCAT
TGGGTTTGACCCCTAATTTTAAATCAAATTTTGATTTGGCAGAAGATGCTAAATTACAGCT
TTCAAAAGATACTTACGATGATGATTTAGATAATTTATTGGCGCAAATTGGAGATCAATA
TGCTGATTTGTTTTTGGCAGCTAAGAATTTATCAGATGCTATTTTACTTTCAGATATCCTA
AGAGTAAATACTGAAATAACTAAGGCTCCCCTATCAGCTTCAATGATTAAACGCTACGAT
GAACATCATCAAGACTTGACTCTTTTAAAAGCTTTAGTTCGACAACAACTTCCAGAAAAG
TATAAAGAAATCTTTTTTGATCAATCAAAAAACGGATATGCAGGTTATATTGATGGGGGA
GCTAGCCAAGAAGAATTTTATAAATTTATCAAACCAATTTTAGAAAAAATGGATGGTACT
GAGGAATTATTGGTGAAACTAAATCGTGAAGATTTGCTGCGCAAGCAACGGACCTTTGAC
AACGGCTCTATTCCCCATCAAATTCACTTGGGTGAGCTGCATGCTATTTTGAGAAGACAA
GAAGACTTTTATCCATTTTTAAAAGACAATCGTGAGAAGATTGAAAAAATCTTGACTTTT
CGAATTCCTTATTATGTTGGTCCATTGGCGCGTGGCAATAGTCGTTTTGCATGGATGACTC
GGAAGTCTGAAGAAACAATTACCCCATGGAATTTTGAAGAAGTTGTCGATAAAGGTGCTT
CAGCTCAATCATTTATTGAACGCATGACAAACTTTGATAAAAATCTTCCAAATGAAAAAG
TACTACCAAAACATAGTTTGCTTTATGAGTATTTTACGGTTTATAACGAATTGACAAAGGT
CAAATATGTTACTGAAGGAATGCGAAAACCAGCATTTCTTTCAGGTGAACAGAAGAAAG
CCATTGTTGATTTACTCTTCAAAACAAATCGAAAAGTAACCGTTAAGCAATTAAAAGAAG
ATTATTTCAAAAAAATAGAATGTTTTGATAGTGTTGAAATTTCAGGAGTTGAAGATAGAT
TTAATGCTTCATTAGGTACCTACCATGATTTGCTAAAAATTATTAAAGATAAAGATTTTTT
GGATAATGAAGAAAATGAAGATATCTTAGAGGATATTGTTTTAACATTGACCTTATTTGA
AGATAGGGAGATGATTGAGGAAAGACTTAAAACATATGCTCACCTCTTTGATGATAAGGT
GATGAAACAGCTTAAACGTCGCCGTTATACTGGTTGGGGACGTTTGTCTCGAAAATTGAT
TAATGGTATTAGGGATAAGCAATCTGGCAAAACAATATTAGATTTTTTGAAATCAGATGG
TTTTGCCAATCGCAATTTTATGCAGCTGATCCATGATGATAGTTTGACATTTAAAGAAGAC
ATTCAAAAAGCACAAGTGTCTGGACAAGGCGATAGTTTACATGAACATATTGCAAATTTA
GCTGGTAGCCCTGCTATTAAAAAAGGTATTTTACAGACTGTAAAAGTTGTTGATGAATTG
GTCAAAGTAATGGGGCGGCATAAGCCAGAAAATATCGTTATTGAAATGGCACGTGAAAA
TCAGACAACTCAAAAGGGCCAGAAAAATTCGCGAGAGCGTATGAAACGAATCGAAGAAG
GTATCAAAGAATTAGGAAGTCAGATTCTTAAAGAGCATCCTGTTGAAAATACTCAATTGC
AAAATGAAAAGCTCTATCTCTATTATCTCCAAAATGGAAGAGACATGTATGTGGACCAAG
AATTAGATATTAATCGTTTAAGTGATTATGATGTCGATCACATTGTTCCACAAAGTTTCCT
TAAAGACGATTCAATAGACAATAAGGTCTTAACGCGTTCTGATAAAAATCGTGGTAAATC
GGATAACGTTCCAAGTGAAGAAGTAGTCAAAAAGATGAAAAACTATTGGAGACAACTTC
TAAACGCCAAGTTAATCACTCAACGTAAGTTTGATAATTTAACGAAAGCTGAACGTGGAG
GTTTGAGTGAACTTGATAAAGCTGGTTTTATCAAACGCCAATTGGTTGAAACTCGCCAAA
TCACTAAGCATGTGGCACAAATTTTGGATAGTCGCATGAATACTAAATACGATGAAAATG
ATAAACTTATTCGAGAGGTTAAAGTGATTACCTTAAAATCTAAATTAGTTTCTGACTTCCG
AAAAGATTTCCAATTCTATAAAGTACGTGAGATTAACAATTACCATCATGCCCATGATGC
GTATCTAAATGCCGTCGTTGGAACTGCTTTGATTAAGAAATATCCAAAACTTGAATCGGA
GTTTGTCTATGGTGATTATAAAGTTTATGATGTTCGTAAAATGATTGCTAAGTCTGAGCAA
GAAATAGGCAAAGCAACCGCAAAATATTTCTTTTACTCTAATATCATGAACTTCTTCAAA
ACAGAAATTACACTTGCAAATGGAGAGATTCGCAAACGCCCTCTAATCGAAACTAATGG
GGAAACTGGAGAAATTGTCTGGGATAAAGGGCGAGATTTTGCCACAGTGCGCAAAGTAT
TGTCCATGCCCCAAGTCAATATTGTCAAGAAAACAGAAGTACAGACAGGCGGATTCTCCA
AGGAGTCAATTTTACCAAAAAGAAATTCGGACAAGCTTATTGCTCGTAAAAAAGACTGG
GATCCAAAAAAATATGGTGGTTTTGATAGTCCAACGGTAGCTTATTCAGTCCTAGTGGTT
GCTAAGGTGGAAAAAGGGAAATCGAAGAAGTTAAAATCCGTTAAAGAGTTACTAGGGAT
CACAATTATGGAAAGAAGTTCCTTTGAAAAAAATCCGATTGACTTTTTAGAAGCTAAAGG
ATATAAGGAAGTTAAAAAAGACTTAATCATTAAACTACCTAAATATAGTCTTTTTGAGTT
AGAAAACGGTCGTAAACGGATGCTGGCTAGTGCCGGAGAATTACAAAAAGGAAATGAGC
TGGCTCTGCCAAGCAAATATGTGAATTTTTTATATTTAGCTAGTCATTATGAAAAGTTGAA
GGGTAGTCCAGAAGATAACGAACAAAAACAATTGTTTGTGGAGCAGCATAAGCATTATTT
AGATGAGATTATTGAGCAAATCAGTGAATTTTCTAAGCGTGTTATTTTAGCAGATGCCAA
TTTAGATAAAGTTCTTAGTGCATATAACAAACATAGAGACAAACCAATACGTGAACAAGC
AGAAAATATTATTCATTTATTTACGTTGACGAATCTTGGAGCTCCCGCTGCTTTTAAATAT
TTTGATACAACAATTGATCGTAAACGATATACGTCTACAAAAGAAGTTTTAGATGCCACT
CTTATCCATCAATCCATCACTGGTCTTTATGAAACACGCATTGATTTGAGTCAGCTAGGAG
GTGACTGA
Sequence for S. pyogenes Cas9_R66A (including N-terminal His-tag), used for in
vivo experiments in E. coli, note that only the coding sequence for the His-tag
differs, the CDS for Cas9 is the same as for the recombinant purified ones;
SEQ ID NO: 13:
atgaaacatcaccatcaccatcacaacactagtCATATCGAAGGTCGTCATAGCGTCGACATGGATAAGAAA
TACTCAATAGGCTTAGATATCGGCACAAATAGCGTCGGATGGGCGGTGATCACTGATGAA
TATAAGGTTCCGTCTAAAAAGTTCAAGGTTCTGGGAAATACAGACCGCCACAGTATCAAA
AAAAATCTTATAGGGGCTCTTTTATTTGACAGTGGAGAGACAGCGGAAGCGACTCGTCTC
AAAGCGACAGCTCGTAGAAGGTATACACGTCGGAAGAATCGTATTTGTTATCTACAGGAG
ATTTTTTCAAATGAGATGGCGAAAGTAGATGATAGTTTCTTTCATCGACTTGAAGAGTCTT
TTTTGGTGGAAGAAGACAAGAAGCATGAACGTCATCCTATTTTTGGAAATATAGTAGATG
AAGTTGCTTATCATGAGAAATATCCAACTATCTATCATCTTCGAAAAAAATTGGTAGATT
CTACTGATAAAGCGGATTTGCGCTTAATCTATTTGGCCTTAGCGCATATGATTAAGTTTCG
TGGTCATTTTTTGATTGAGGGAGATTTAAATCCTGATAATAGTGATGTGGACAAACTATTT
ATCCAGTTGGTACAAACCTACAATCAATTATTTGAAGAAAACCCTATTAACGCAAGTGGA
GTAGATGCTAAAGCGATTCTTTCTGCACGATTGAGTAAATCAAGACGATTAGAAAATCTC
ATTGCTCAGCTCCCCGGTGAGAAGAAAAATGGCTTATTTGGGAATCTCATTGCTTTGTCAT
TGGGTTTGACCCCTAATTTTAAATCAAATTTTGATTTGGCAGAAGATGCTAAATTACAGCT
TTCAAAAGATACTTACGATGATGATTTAGATAATTTATTGGCGCAAATTGGAGATCAATA
TGCTGATTTGTTTTTGGCAGCTAAGAATTTATCAGATGCTATTTTACTTTCAGATATCCTA
AGAGTAAATACTGAAATAACTAAGGCTCCCCTATCAGCTTCAATGATTAAACGCTACGAT
GAACATCATCAAGACTTGACTCTTTTAAAAGCTTTAGTTCGACAACAACTTCCAGAAAAG
TATAAAGAAATCTTTTTTGATCAATCAAAAAACGGATATGCAGGTTATATTGATGGGGGA
GCTAGCCAAGAAGAATTTTATAAATTTATCAAACCAATTTTAGAAAAAATGGATGGTACT
GAGGAATTATTGGTGAAACTAAATCGTGAAGATTTGCTGCGCAAGCAACGGACCTTTGAC
AACGGCTCTATTCCCCATCAAATTCACTTGGGTGAGCTGCATGCTATTTTGAGAAGACAA
GAAGACTTTTATCCATTTTTAAAAGACAATCGTGAGAAGATTGAAAAAATCTTGACTTTT
CGAATTCCTTATTATGTTGGTCCATTGGCGCGTGGCAATAGTCGTTTTGCATGGATGACTC
GGAAGTCTGAAGAAACAATTACCCCATGGAATTTTGAAGAAGTTGTCGATAAAGGTGCTT
CAGCTCAATCATTTATTGAACGCATGACAAACTTTGATAAAAATCTTCCAAATGAAAAAG
TACTACCAAAACATAGTTTGCTTTATGAGTATTTTACGGTTTATAACGAATTGACAAAGGT
CAAATATGTTACTGAAGGAATGCGAAAACCAGCATTTCTTTCAGGTGAACAGAAGAAAG
CCATTGTTGATTTACTCTTCAAAACAAATCGAAAAGTAACCGTTAAGCAATTAAAAGAAG
ATTATTTCAAAAAAATAGAATGTTTTGATAGTGTTGAAATTTCAGGAGTTGAAGATAGAT
TTAATGCTTCATTAGGTACCTACCATGATTTGCTAAAAATTATTAAAGATAAAGATTTTTT
GGATAATGAAGAAAATGAAGATATCTTAGAGGATATTGTTTTAACATTGACCTTATTTGA
AGATAGGGAGATGATTGAGGAAAGACTTAAAACATATGCTCACCTCTTTGATGATAAGGT
GATGAAACAGCTTAAACGTCGCCGTTATACTGGTTGGGGACGTTTGTCTCGAAAATTGAT
TAATGGTATTAGGGATAAGCAATCTGGCAAAACAATATTAGATTTTTTGAAATCAGATGG
TTTTGCCAATCGCAATTTTATGCAGCTGATCCATGATGATAGTTTGACATTTAAAGAAGAC
ATTCAAAAAGCACAAGTGTCTGGACAAGGCGATAGTTTACATGAACATATTGCAAATTTA
GCTGGTAGCCCTGCTATTAAAAAAGGTATTTTACAGACTGTAAAAGTTGTTGATGAATTG
GTCAAAGTAATGGGGCGGCATAAGCCAGAAAATATCGTTATTGAAATGGCACGTGAAAA
TCAGACAACTCAAAAGGGCCAGAAAAATTCGCGAGAGCGTATGAAACGAATCGAAGAAG
GTATCAAAGAATTAGGAAGTCAGATTCTTAAAGAGCATCCTGTTGAAAATACTCAATTGC
AAAATGAAAAGCTCTATCTCTATTATCTCCAAAATGGAAGAGACATGTATGTGGACCAAG
AATTAGATATTAATCGTTTAAGTGATTATGATGTCGATCACATTGTTCCACAAAGTTTCCT
TAAAGACGATTCAATAGACAATAAGGTCTTAACGCGTTCTGATAAAAATCGTGGTAAATC
GGATAACGTTCCAAGTGAAGAAGTAGTCAAAAAGATGAAAAACTATTGGAGACAACTTC
TAAACGCCAAGTTAATCACTCAACGTAAGTTTGATAATTTAACGAAAGCTGAACGTGGAG
GTTTGAGTGAACTTGATAAAGCTGGTTTTATCAAACGCCAATTGGTTGAAACTCGCCAAA
TCACTAAGCATGTGGCACAAATTTTGGATAGTCGCATGAATACTAAATACGATGAAAATG
ATAAACTTATTCGAGAGGTTAAAGTGATTACCTTAAAATCTAAATTAGTTTCTGACTTCCG
AAAAGATTTCCAATTCTATAAAGTACGTGAGATTAACAATTACCATCATGCCCATGATGC
GTATCTAAATGCCGTCGTTGGAACTGCTTTGATTAAGAAATATCCAAAACTTGAATCGGA
GTTTGTCTATGGTGATTATAAAGTTTATGATGTTCGTAAAATGATTGCTAAGTCTGAGCAA
GAAATAGGCAAAGCAACCGCAAAATATTTCTTTTACTCTAATATCATGAACTTCTTCAAA
ACAGAAATTACACTTGCAAATGGAGAGATTCGCAAACGCCCTCTAATCGAAACTAATGG
GGAAACTGGAGAAATTGTCTGGGATAAAGGGCGAGATTTTGCCACAGTGCGCAAAGTAT
TGTCCATGCCCCAAGTCAATATTGTCAAGAAAACAGAAGTACAGACAGGCGGATTCTCCA
AGGAGTCAATTTTACCAAAAAGAAATTCGGACAAGCTTATTGCTCGTAAAAAAGACTGG
GATCCAAAAAAATATGGTGGTTTTGATAGTCCAACGGTAGCTTATTCAGTCCTAGTGGTT
GCTAAGGTGGAAAAAGGGAAATCGAAGAAGTTAAAATCCGTTAAAGAGTTACTAGGGAT
CACAATTATGGAAAGAAGTTCCTTTGAAAAAAATCCGATTGACTTTTTAGAAGCTAAAGG
ATATAAGGAAGTTAAAAAAGACTTAATCATTAAACTACCTAAATATAGTCTTTTTGAGTT
AGAAAACGGTCGTAAACGGATGCTGGCTAGTGCCGGAGAATTACAAAAAGGAAATGAGC
TGGCTCTGCCAAGCAAATATGTGAATTTTTTATATTTAGCTAGTCATTATGAAAAGTTGAA
GGGTAGTCCAGAAGATAACGAACAAAAACAATTGTTTGTGGAGCAGCATAAGCATTATTT
AGATGAGATTATTGAGCAAATCAGTGAATTTTCTAAGCGTGTTATTTTAGCAGATGCCAA
TTTAGATAAAGTTCTTAGTGCATATAACAAACATAGAGACAAACCAATACGTGAACAAGC
AGAAAATATTATTCATTTATTTACGTTGACGAATCTTGGAGCTCCCGCTGCTTTTAAATAT
TTTGATACAACAATTGATCGTAAACGATATACGTCTACAAAAGAAGTTTTAGATGCCACT
CTTATCCATCAATCCATCACTGGTCTTTATGAAACACGCATTGATTTGAGTCAGCTAGGAG
GTGACTGA
Sequence for S. pyogenes Cas9_R70A (including N-terminal His-tag), used for in
vivo experiments in E. coli, note that only the coding sequence for the His-tag
differs, the CDS for Cas9 is the same as for the recombinant purified ones;
SEQ ID NO: 14:
atgaaacatcaccatcaccatcacaacactagtCATATCGAAGGTCGTCATAGCGTCGACATGGATAAGAAA
TACTCAATAGGCTTAGATATCGGCACAAATAGCGTCGGATGGGCGGTGATCACTGATGAA
TATAAGGTTCCGTCTAAAAAGTTCAAGGTTCTGGGAAATACAGACCGCCACAGTATCAAA
AAAAATCTTATAGGGGCTCTTTTATTTGACAGTGGAGAGACAGCGGAAGCGACTCGTCTC
AAACGGACAGCTCGTGCAAGGTATACACGTCGGAAGAATCGTATTTGTTATCTACAGGAG
ATTTTTTCAAATGAGATGGCGAAAGTAGATGATAGTTTCTTTCATCGACTTGAAGAGTCTT
TTTTGGTGGAAGAAGACAAGAAGCATGAACGTCATCCTATTTTTGGAAATATAGTAGATG
AAGTTGCTTATCATGAGAAATATCCAACTATCTATCATCTTCGAAAAAAATTGGTAGATT
CTACTGATAAAGCGGATTTGCGCTTAATCTATTTGGCCTTAGCGCATATGATTAAGTTTCG
TGGTCATTTTTTGATTGAGGGAGATTTAAATCCTGATAATAGTGATGTGGACAAACTATTT
ATCCAGTTGGTACAAACCTACAATCAATTATTTGAAGAAAACCCTATTAACGCAAGTGGA
GTAGATGCTAAAGCGATTCTTTCTGCACGATTGAGTAAATCAAGACGATTAGAAAATCTC
ATTGCTCAGCTCCCCGGTGAGAAGAAAAATGGCTTATTTGGGAATCTCATTGCTTTGTCAT
TGGGTTTGACCCCTAATTTTAAATCAAATTTTGATTTGGCAGAAGATGCTAAATTACAGCT
TTCAAAAGATACTTACGATGATGATTTAGATAATTTATTGGCGCAAATTGGAGATCAATA
TGCTGATTTGTTTTTGGCAGCTAAGAATTTATCAGATGCTATTTTACTTTCAGATATCCTA
AGAGTAAATACTGAAATAACTAAGGCTCCCCTATCAGCTTCAATGATTAAACGCTACGAT
GAACATCATCAAGACTTGACTCTTTTAAAAGCTTTAGTTCGACAACAACTTCCAGAAAAG
TATAAAGAAATCTTTTTTGATCAATCAAAAAACGGATATGCAGGTTATATTGATGGGGGA
GCTAGCCAAGAAGAATTTTATAAATTTATCAAACCAATTTTAGAAAAAATGGATGGTACT
GAGGAATTATTGGTGAAACTAAATCGTGAAGATTTGCTGCGCAAGCAACGGACCTTTGAC
AACGGCTCTATTCCCCATCAAATTCACTTGGGTGAGCTGCATGCTATTTTGAGAAGACAA
GAAGACTTTTATCCATTTTTAAAAGACAATCGTGAGAAGATTGAAAAAATCTTGACTTTT
CGAATTCCTTATTATGTTGGTCCATTGGCGCGTGGCAATAGTCGTTTTGCATGGATGACTC
GGAAGTCTGAAGAAACAATTACCCCATGGAATTTTGAAGAAGTTGTCGATAAAGGTGCTT
CAGCTCAATCATTTATTGAACGCATGACAAACTTTGATAAAAATCTTCCAAATGAAAAAG
TACTACCAAAACATAGTTTGCTTTATGAGTATTTTACGGTTTATAACGAATTGACAAAGGT
CAAATATGTTACTGAAGGAATGCGAAAACCAGCATTTCTTTCAGGTGAACAGAAGAAAG
CCATTGTTGATTTACTCTTCAAAACAAATCGAAAAGTAACCGTTAAGCAATTAAAAGAAG
ATTATTTCAAAAAAATAGAATGTTTTGATAGTGTTGAAATTTCAGGAGTTGAAGATAGAT
TTAATGCTTCATTAGGTACCTACCATGATTTGCTAAAAATTATTAAAGATAAAGATTTTTT
GGATAATGAAGAAAATGAAGATATCTTAGAGGATATTGTTTTAACATTGACCTTATTTGA
AGATAGGGAGATGATTGAGGAAAGACTTAAAACATATGCTCACCTCTTTGATGATAAGGT
GATGAAACAGCTTAAACGTCGCCGTTATACTGGTTGGGGACGTTTGTCTCGAAAATTGAT
TAATGGTATTAGGGATAAGCAATCTGGCAAAACAATATTAGATTTTTTGAAATCAGATGG
TTTTGCCAATCGCAATTTTATGCAGCTGATCCATGATGATAGTTTGACATTTAAAGAAGAC
ATTCAAAAAGCACAAGTGTCTGGACAAGGCGATAGTTTACATGAACATATTGCAAATTTA
GCTGGTAGCCCTGCTATTAAAAAAGGTATTTTACAGACTGTAAAAGTTGTTGATGAATTG
GTCAAAGTAATGGGGCGGCATAAGCCAGAAAATATCGTTATTGAAATGGCACGTGAAAA
TCAGACAACTCAAAAGGGCCAGAAAAATTCGCGAGAGCGTATGAAACGAATCGAAGAAG
GTATCAAAGAATTAGGAAGTCAGATTCTTAAAGAGCATCCTGTTGAAAATACTCAATTGC
AAAATGAAAAGCTCTATCTCTATTATCTCCAAAATGGAAGAGACATGTATGTGGACCAAG
AATTAGATATTAATCGTTTAAGTGATTATGATGTCGATCACATTGTTCCACAAAGTTTCCT
TAAAGACGATTCAATAGACAATAAGGTCTTAACGCGTTCTGATAAAAATCGTGGTAAATC
GGATAACGTTCCAAGTGAAGAAGTAGTCAAAAAGATGAAAAACTATTGGAGACAACTTC
TAAACGCCAAGTTAATCACTCAACGTAAGTTTGATAATTTAACGAAAGCTGAACGTGGAG
GTTTGAGTGAACTTGATAAAGCTGGTTTTATCAAACGCCAATTGGTTGAAACTCGCCAAA
TCACTAAGCATGTGGCACAAATTTTGGATAGTCGCATGAATACTAAATACGATGAAAATG
ATAAACTTATTCGAGAGGTTAAAGTGATTACCTTAAAATCTAAATTAGTTTCTGACTTCCG
AAAAGATTTCCAATTCTATAAAGTACGTGAGATTAACAATTACCATCATGCCCATGATGC
GTATCTAAATGCCGTCGTTGGAACTGCTTTGATTAAGAAATATCCAAAACTTGAATCGGA
GTTTGTCTATGGTGATTATAAAGTTTATGATGTTCGTAAAATGATTGCTAAGTCTGAGCAA
GAAATAGGCAAAGCAACCGCAAAATATTTCTTTTACTCTAATATCATGAACTTCTTCAAA
ACAGAAATTACACTTGCAAATGGAGAGATTCGCAAACGCCCTCTAATCGAAACTAATGG
GGAAACTGGAGAAATTGTCTGGGATAAAGGGCGAGATTTTGCCACAGTGCGCAAAGTAT
TGTCCATGCCCCAAGTCAATATTGTCAAGAAAACAGAAGTACAGACAGGCGGATTCTCCA
AGGAGTCAATTTTACCAAAAAGAAATTCGGACAAGCTTATTGCTCGTAAAAAAGACTGG
GATCCAAAAAAATATGGTGGTTTTGATAGTCCAACGGTAGCTTATTCAGTCCTAGTGGTT
GCTAAGGTGGAAAAAGGGAAATCGAAGAAGTTAAAATCCGTTAAAGAGTTACTAGGGAT
CACAATTATGGAAAGAAGTTCCTTTGAAAAAAATCCGATTGACTTTTTAGAAGCTAAAGG
ATATAAGGAAGTTAAAAAAGACTTAATCATTAAACTACCTAAATATAGTCTTTTTGAGTT
AGAAAACGGTCGTAAACGGATGCTGGCTAGTGCCGGAGAATTACAAAAAGGAAATGAGC
TGGCTCTGCCAAGCAAATATGTGAATTTTTTATATTTAGCTAGTCATTATGAAAAGTTGAA
GGGTAGTCCAGAAGATAACGAACAAAAACAATTGTTTGTGGAGCAGCATAAGCATTATTT
AGATGAGATTATTGAGCAAATCAGTGAATTTTCTAAGCGTGTTATTTTAGCAGATGCCAA
TTTAGATAAAGTTCTTAGTGCATATAACAAACATAGAGACAAACCAATACGTGAACAAGC
AGAAAATATTATTCATTTATTTACGTTGACGAATCTTGGAGCTCCCGCTGCTTTTAAATAT
TTTGATACAACAATTGATCGTAAACGATATACGTCTACAAAAGAAGTTTTAGATGCCACT
CTTATCCATCAATCCATCACTGGTCTTTATGAAACACGCATTGATTTGAGTCAGCTAGGAG
GTGACTGA
Sequence for S. pyogenes Cas9 Q768A (including N-terminal His-tag), used for in
vivo experiments in E. coli, note that only the coding sequence for the His-tag
differs, the CDS for Cas9 is the same as for the recombinant purified ones;
SEQ ID NO: 15:
atgaaacatcaccatcaccatcacaacactagtCATATCGAAGGTCGTCATAGCGTCGACATGGATAAGAAA
TACTCAATAGGCTTAGATATCGGCACAAATAGCGTCGGATGGGCGGTGATCACTGATGAA
TATAAGGTTCCGTCTAAAAAGTTCAAGGTTCTGGGAAATACAGACCGCCACAGTATCAAA
AAAAATCTTATAGGGGCTCTTTTATTTGACAGTGGAGAGACAGCGGAAGCGACTCGTCTC
AAACGGACAGCTCGTAGAAGGTATACACGTCGGAAGAATCGTATTTGTTATCTACAGGAG
ATTTTTTCAAATGAGATGGCGAAAGTAGATGATAGTTTCTTTCATCGACTTGAAGAGTCTT
TTTTGGTGGAAGAAGACAAGAAGCATGAACGTCATCCTATTTTTGGAAATATAGTAGATG
AAGTTGCTTATCATGAGAAATATCCAACTATCTATCATCTGCGAAAAAAATTGGTAGATT
CTACTGATAAAGCGGATTTGCGCTTAATCTATTTGGCCTTAGCGCATATGATTAAGTTTCG
TGGTCATTTTTTGATTGAGGGAGATTTAAATCCTGATAATAGTGATGTGGACAAACTATTT
ATCCAGTTGGTACAAACCTACAATCAATTATTTGAAGAAAACCCTATTAACGCAAGTGGA
GTAGATGCTAAAGCGATTCTTTCTGCACGATTGAGTAAATCAAGACGATTAGAAAATCTC
ATTGCTCAGCTCCCCGGTGAGAAGAAAAATGGCTTATTTGGGAATCTCATTGCTTTGTCAT
TGGGTTTGACCCCTAATTTTAAATCAAATTTTGATTTGGCAGAAGATGCTAAATTACAGCT
TTCAAAAGATACTTACGATGATGATTTAGATAATTTATTGGCGCAAATTGGAGATCAATA
TGCTGATTTGTTTTTGGCAGCTAAGAATTTATCAGATGCTATTTTACTTTCAGATATCCTA
AGAGTAAATACTGAAATAACTAAGGCTCCCCTATCAGCTTCAATGATTAAACGCTACGAT
GAACATCATCAAGACTTGACTCTTTTAAAAGCTTTAGTTCGACAACAACTTCCAGAAAAG
TATAAAGAAATCTTTTTTGATCAATCAAAAAACGGATATGCAGGTTATATTGATGGGGGA
GCTAGCCAAGAAGAATTTTATAAATTTATCAAACCAATTTTAGAAAAAATGGATGGTACT
GAGGAATTATTGGTGAAACTAAATCGTGAAGATTTGCTGCGCAAGCAACGGACCTTTGAC
AACGGCTCTATTCCCCATCAAATTCACTTGGGTGAGCTGCATGCTATTTTGAGAAGACAA
GAAGACTTTTATCCATTTTTAAAAGACAATCGTGAGAAGATTGAAAAAATCTTGACTTTT
CGAATTCCTTATTATGTTGGTCCATTGGCGCGTGGCAATAGTCGTTTTGCATGGATGACTC
GGAAGTCTGAAGAAACAATTACCCCATGGAATTTTGAAGAAGTTGTCGATAAAGGTGCTT
CAGCTCAATCATTTATTGAACGCATGACAAACTTTGATAAAAATCTTCCAAATGAAAAAG
TACTACCAAAACATAGTTTGCTTTATGAGTATTTTACGGTTTATAACGAATTGACAAAGGT
CAAATATGTTACTGAAGGAATGCGAAAACCAGCATTTCTTTCAGGTGAACAGAAGAAAG
CCATTGTTGATTTACTCTTCAAAACAAATCGAAAAGTAACCGTTAAGCAATTAAAAGAAG
ATTATTTCAAAAAAATAGAATGTTTTGATAGTGTTGAAATTTCAGGAGTTGAAGATAGAT
TTAATGCTTCATTAGGTACCTACCATGATTTGCTAAAAATTATTAAAGATAAAGATTTTTT
GGATAATGAAGAAAATGAAGATATCTTAGAGGATATTGTTTTAACATTGACCTTATTTGA
AGATAGGGAGATGATTGAGGAAAGACTTAAAACATATGCTCACCTCTTTGATGATAAGGT
GATGAAACAGCTTAAACGTCGCCGTTATACTGGTTGGGGACGTTTGTCTCGAAAATTGAT
TAATGGTATTAGGGATAAGCAATCTGGCAAAACAATATTAGATTTTTTGAAATCAGATGG
TTTTGCCAATCGCAATTTTATGCAGCTGATCCATGATGATAGTTTGACATTTAAAGAAGAC
ATTCAAAAAGCACAAGTGTCTGGACAAGGCGATAGTTTACATGAACATATTGCAAATTTA
GCTGGTAGCCCTGCTATTAAAAAAGGTATTTTACAGACTGTAAAAGTTGTTGATGAATTG
GTCAAAGTAATGGGGCGGCATAAGCCAGAAAATATCGTTATTGAAATGGCACGTGAAAA
TGCGACAACTCAAAAGGGCCAGAAAAATTCGCGAGAGCGTATGAAACGAATCGAAGAAG
GTATCAAAGAATTAGGAAGTCAGATTCTTAAAGAGCATCCTGTTGAAAATACTCAATTGC
AAAATGAAAAGCTCTATCTCTATTATCTCCAAAATGGAAGAGACATGTATGTGGACCAAG
AATTAGATATTAATCGTTTAAGTGATTATGATGTCGATCACATTGTTCCACAAAGTTTCCT
TAAAGACGATTCAATAGACAATAAGGTCTTAACGCGTTCTGATAAAAATCGTGGTAAATC
GGATAACGTTCCAAGTGAAGAAGTAGTCAAAAAGATGAAAAACTATTGGAGACAACTTC
TAAACGCCAAGTTAATCACTCAACGTAAGTTTGATAATTTAACGAAAGCTGAACGTGGAG
GTTTGAGTGAACTTGATAAAGCTGGTTTTATCAAACGCCAATTGGTTGAAACTCGCCAAA
TCACTAAGCATGTGGCACAAATTTTGGATAGTCGCATGAATACTAAATACGATGAAAATG
ATAAACTTATTCGAGAGGTTAAAGTGATTACCTTAAAATCTAAATTAGTTTCTGACTTCCG
AAAAGATTTCCAATTCTATAAAGTACGTGAGATTAACAATTACCATCATGCCCATGATGC
GTATCTAAATGCCGTCGTTGGAACTGCTTTGATTAAGAAATATCCAAAACTTGAATCGGA
GTTTGTCTATGGTGATTATAAAGTTTATGATGTTCGTAAAATGATTGCTAAGTCTGAGCAA
GAAATAGGCAAAGCAACCGCAAAATATTTCTTTTACTCTAATATCATGAACTTCTTCAAA
ACAGAAATTACACTTGCAAATGGAGAGATTCGCAAACGCCCTCTAATCGAAACTAATGG
GGAAACTGGAGAAATTGTCTGGGATAAAGGGCGAGATTTTGCCACAGTGCGCAAAGTAT
TGTCCATGCCCCAAGTCAATATTGTCAAGAAAACAGAAGTACAGACAGGCGGATTCTCCA
AGGAGTCAATTTTACCAAAAAGAAATTCGGACAAGCTTATTGCTCGTAAAAAAGACTGG
GATCCAAAAAAATATGGTGGTTTTGATAGTCCAACGGTAGCTTATTCAGTCCTAGTGGTT
GCTAAGGTGGAAAAAGGGAAATCGAAGAAGTTAAAATCCGTTAAAGAGTTACTAGGGAT
CACAATTATGGAAAGAAGTTCCTTTGAAAAAAATCCGATTGACTTTTTAGAAGCTAAAGG
ATATAAGGAAGTTAAAAAAGACTTAATCATTAAACTACCTAAATATAGTCTTTTTGAGTT
AGAAAACGGTCGTAAACGGATGCTGGCTAGTGCCGGAGAATTACAAAAAGGAAATGAGC
TGGCTCTGCCAAGCAAATATGTGAATTTTTTATATTTAGCTAGTCATTATGAAAAGTTGAA
GGGTAGTCCAGAAGATAACGAACAAAAACAATTGTTTGTGGAGCAGCATAAGCATTATTT
AGATGAGATTATTGAGCAAATCAGTGAATTTTCTAAGCGTGTTATTTTAGCAGATGCCAA
TTTAGATAAAGTTCTTAGTGCATATAACAAACATAGAGACAAACCAATACGTGAACAAGC
AGAAAATATTATTCATTTATTTACGTTGACGAATCTTGGAGCTCCCGCTGCTTTTAAATAT
TTTGATACAACAATTGATCGTAAACGATATACGTCTACAAAAGAAGTTTTAGATGCCACT
CTTATCCATCAATCCATCACTGGTCTTTATGAAACACGCATTGATTTGAGTCAGCTAGGAG
GTGACTGA
Sequence for S. pyogenes Cas9_R63A/Q768A (including N-terminal His-tag), used for in
vivo experiments in E. coli, note that only the coding sequence for the His-tag
differs, the CDS for Cas9 is the same as for the recombinant purified ones;
SEQ ID NO: 16:
atgaaacatcaccatcaccatcacaacactagtCATATCGAAGGTCGTCATAGCGTCGACATGGATAAGAAA
TACTCAATAGGCTTAGATATCGGCACAAATAGCGTCGGATGGGCGGTGATCACTGATGAA
TATAAGGTTCCGTCTAAAAAGTTCAAGGTTCTGGGAAATACAGACCGCCACAGTATCAAA
AAAAATCTTATAGGGGCTCTTTTATTTGACAGTGGAGAGACAGCGGAAGCGACTGCTCTC
AAACGGACAGCTCGTAGAAGGTATACACGTCGGAAGAATCGTATTTGTTATCTACAGGAG
ATTTTTTCAAATGAGATGGCGAAAGTAGATGATAGTTTCTTTCATCGACTTGAAGAGTCTT
TTTTGGTGGAAGAAGACAAGAAGCATGAACGTCATCCTATTTTTGGAAATATAGTAGATG
AAGTTGCTTATCATGAGAAATATCCAACTATCTATCATCTTCGAAAAAAATTGGTAGATT
CTACTGATAAAGCGGATTTGCGCTTAATCTATTTGGCCTTAGCGCATATGATTAAGTTTCG
TGGTCATTTTTTGATTGAGGGAGATTTAAATCCTGATAATAGTGATGTGGACAAACTATTT
ATCCAGTTGGTACAAACCTACAATCAATTATTTGAAGAAAACCCTATTAACGCAAGTGGA
GTAGATGCTAAAGCGATTCTTTCTGCACGATTGAGTAAATCAAGACGATTAGAAAATCTC
ATTGCTCAGCTCCCCGGTGAGAAGAAAAATGGCTTATTTGGGAATCTCATTGCTTTGTCAT
TGGGTTTGACCCCTAATTTTAAATCAAATTTTGATTTGGCAGAAGATGCTAAATTACAGCT
TTCAAAAGATACTTACGATGATGATTTAGATAATTTATTGGCGCAAATTGGAGATCAATA
TGCTGATTTGTTTTTGGCAGCTAAGAATTTATCAGATGCTATTTTACTTTCAGATATCCTA
AGAGTAAATACTGAAATAACTAAGGCTCCCCTATCAGCTTCAATGATTAAACGCTACGAT
GAACATCATCAAGACTTGACTCTTTTAAAAGCTTTAGTTCGACAACAACTTCCAGAAAAG
TATAAAGAAATCTTTTTTGATCAATCAAAAAACGGATATGCAGGTTATATTGATGGGGGA
GCTAGCCAAGAAGAATTTTATAAATTTATCAAACCAATTTTAGAAAAAATGGATGGTACT
GAGGAATTATTGGTGAAACTAAATCGTGAAGATTTGCTGCGCAAGCAACGGACCTTTGAC
AACGGCTCTATTCCCCATCAAATTCACTTGGGTGAGCTGCATGCTATTTTGAGAAGACAA
GAAGACTTTTATCCATTTTTAAAAGACAATCGTGAGAAGATTGAAAAAATCTTGACTTTT
CGAATTCCTTATTATGTTGGTCCATTGGCGCGTGGCAATAGTCGTTTTGCATGGATGACTC
GGAAGTCTGAAGAAACAATTACCCCATGGAATTTTGAAGAAGTTGTCGATAAAGGTGCTT
CAGCTCAATCATTTATTGAACGCATGACAAACTTTGATAAAAATCTTCCAAATGAAAAAG
TACTACCAAAACATAGTTTGCTTTATGAGTATTTTACGGTTTATAACGAATTGACAAAGGT
CAAATATGTTACTGAAGGAATGCGAAAACCAGCATTTCTTTCAGGTGAACAGAAGAAAG
CCATTGTTGATTTACTCTTCAAAACAAATCGAAAAGTAACCGTTAAGCAATTAAAAGAAG
ATTATTTCAAAAAAATAGAATGTTTTGATAGTGTTGAAATTTCAGGAGTTGAAGATAGAT
TTAATGCTTCATTAGGTACCTACCATGATTTGCTAAAAATTATTAAAGATAAAGATTTTTT
GGATAATGAAGAAAATGAAGATATCTTAGAGGATATTGTTTTAACATTGACCTTATTTGA
AGATAGGGAGATGATTGAGGAAAGACTTAAAACATATGCTCACCTCTTTGATGATAAGGT
GATGAAACAGCTTAAACGTCGCCGTTATACTGGTTGGGGACGTTTGTCTCGAAAATTGAT
TAATGGTATTAGGGATAAGCAATCTGGCAAAACAATATTAGATTTTTTGAAATCAGATGG
TTTTGCCAATCGCAATTTTATGCAGCTGATCCATGATGATAGTTTGACATTTAAAGAAGAC
ATTCAAAAAGCACAAGTGTCTGGACAAGGCGATAGTTTACATGAACATATTGCAAATTTA
GCTGGTAGCCCTGCTATTAAAAAAGGTATTTTACAGACTGTAAAAGTTGTTGATGAATTG
GTCAAAGTAATGGGGCGGCATAAGCCAGAAAATATCGTTATTGAAATGGCACGTGAAAA
TGCGACAACTCAAAAGGGCCAGAAAAATTCGCGAGAGCGTATGAAACGAATCGAAGAAG
GTATCAAAGAATTAGGAAGTCAGATTCTTAAAGAGCATCCTGTTGAAAATACTCAATTGC
AAAATGAAAAGCTCTATCTCTATTATCTCCAAAATGGAAGAGACATGTATGTGGACCAAG
AATTAGATATTAATCGTTTAAGTGATTATGATGTCGATCACATTGTTCCACAAAGTTTCCT
TAAAGACGATTCAATAGACAATAAGGTCTTAACGCGTTCTGATAAAAATCGTGGTAAATC
GGATAACGTTCCAAGTGAAGAAGTAGTCAAAAAGATGAAAAACTATTGGAGACAACTTC
TAAACGCCAAGTTAATCACTCAACGTAAGTTTGATAATTTAACGAAAGCTGAACGTGGAG
GTTTGAGTGAACTTGATAAAGCTGGTTTTATCAAACGCCAATTGGTTGAAACTCGCCAAA
TCACTAAGCATGTGGCACAAATTTTGGATAGTCGCATGAATACTAAATACGATGAAAATG
ATAAACTTATTCGAGAGGTTAAAGTGATTACCTTAAAATCTAAATTAGTTTCTGACTTCCG
AAAAGATTTCCAATTCTATAAAGTACGTGAGATTAACAATTACCATCATGCCCATGATGC
GTATCTAAATGCCGTCGTTGGAACTGCTTTGATTAAGAAATATCCAAAACTTGAATCGGA
GTTTGTCTATGGTGATTATAAAGTTTATGATGTTCGTAAAATGATTGCTAAGTCTGAGCAA
GAAATAGGCAAAGCAACCGCAAAATATTTCTTTTACTCTAATATCATGAACTTCTTCAAA
ACAGAAATTACACTTGCAAATGGAGAGATTCGCAAACGCCCTCTAATCGAAACTAATGG
GGAAACTGGAGAAATTGTCTGGGATAAAGGGCGAGATTTTGCCACAGTGCGCAAAGTAT
TGTCCATGCCCCAAGTCAATATTGTCAAGAAAACAGAAGTACAGACAGGCGGATTCTCCA
AGGAGTCAATTTTACCAAAAAGAAATTCGGACAAGCTTATTGCTCGTAAAAAAGACTGG
GATCCAAAAAAATATGGTGGTTTTGATAGTCCAACGGTAGCTTATTCAGTCCTAGTGGTT
GCTAAGGTGGAAAAAGGGAAATCGAAGAAGTTAAAATCCGTTAAAGAGTTACTAGGGAT
CACAATTATGGAAAGAAGTTCCTTTGAAAAAAATCCGATTGACTTTTTAGAAGCTAAAGG
ATATAAGGAAGTTAAAAAAGACTTAATCATTAAACTACCTAAATATAGTCTTTTTGAGTT
AGAAAACGGTCGTAAACGGATGCTGGCTAGTGCCGGAGAATTACAAAAAGGAAATGAGC
TGGCTCTGCCAAGCAAATATGTGAATTTTTTATATTTAGCTAGTCATTATGAAAAGTTGAA
GGGTAGTCCAGAAGATAACGAACAAAAACAATTGTTTGTGGAGCAGCATAAGCATTATTT
AGATGAGATTATTGAGCAAATCAGTGAATTTTCTAAGCGTGTTATTTTAGCAGATGCCAA
TTTAGATAAAGTTCTTAGTGCATATAACAAACATAGAGACAAACCAATACGTGAACAAGC
AGAAAATATTATTCATTTATTTACGTTGACGAATCTTGGAGCTCCCGCTGCTTTTAAATAT
TTTGATACAACAATTGATCGTAAACGATATACGTCTACAAAAGAAGTTTTAGATGCCACT
CTTATCCATCAATCCATCACTGGTCTTTATGAAACACGCATTGATTTGAGTCAGCTAGGAG
GTGACTGA
Sequence for S. pyogenes Cas9_R66A/Q768A (including N-terminal His-tag), used for in
vivo experiments in E. coli, note that only the coding sequence for the His-tag
differs, the CDS for Cas9 is the same as for the recombinant purified ones;
SEQ ID NO: 17:
atgaaacatcaccatcaccatcacaacactagtCATATCGAAGGTCGTCATAGCGTCGACATGGATAAGAAA
TACTCAATAGGCTTAGATATCGGCACAAATAGCGTCGGATGGGCGGTGATCACTGATGAA
TATAAGGTTCCGTCTAAAAAGTTCAAGGTTCTGGGAAATACAGACCGCCACAGTATCAAA
AAAAATCTTATAGGGGCTCTTTTATTTGACAGTGGAGAGACAGCGGAAGCGACTCGTCTC
AAAGCGACAGCTCGTAGAAGGTATACACGTCGGAAGAATCGTATTTGTTATCTACAGGAG
ATTTTTTCAAATGAGATGGCGAAAGTAGATGATAGTTTCTTTCATCGACTTGAAGAGTCTT
TTTTGGTGGAAGAAGACAAGAAGCATGAACGTCATCCTATTTTTGGAAATATAGTAGATG
AAGTTGCTTATCATGAGAAATATCCAACTATCTATCATCTTCGAAAAAAATTGGTAGATT
CTACTGATAAAGCGGATTTGCGCTTAATCTATTTGGCCTTAGCGCATATGATTAAGTTTCG
TGGTCATTTTTTGATTGAGGGAGATTTAAATCCTGATAATAGTGATGTGGACAAACTATTT
ATCCAGTTGGTACAAACCTACAATCAATTATTTGAAGAAAACCCTATTAACGCAAGTGGA
GTAGATGCTAAAGCGATTCTTTCTGCACGATTGAGTAAATCAAGACGATTAGAAAATCTC
ATTGCTCAGCTCCCCGGTGAGAAGAAAAATGGCTTATTTGGGAATCTCATTGCTTTGTCAT
TGGGTTTGACCCCTAATTTTAAATCAAATTTTGATTTGGCAGAAGATGCTAAATTACAGCT
TTCAAAAGATACTTACGATGATGATTTAGATAATTTATTGGCGCAAATTGGAGATCAATA
TGCTGATTTGTTTTTGGCAGCTAAGAATTTATCAGATGCTATTTTACTTTCAGATATCCTA
AGAGTAAATACTGAAATAACTAAGGCTCCCCTATCAGCTTCAATGATTAAACGCTACGAT
GAACATCATCAAGACTTGACTCTTTTAAAAGCTTTAGTTCGACAACAACTTCCAGAAAAG
TATAAAGAAATCTTTTTTGATCAATCAAAAAACGGATATGCAGGTTATATTGATGGGGGA
GCTAGCCAAGAAGAATTTTATAAATTTATCAAACCAATTTTAGAAAAAATGGATGGTACT
GAGGAATTATTGGTGAAACTAAATCGTGAAGATTTGCTGCGCAAGCAACGGACCTTTGAC
AACGGCTCTATTCCCCATCAAATTCACTTGGGTGAGCTGCATGCTATTTTGAGAAGACAA
GAAGACTTTTATCCATTTTTAAAAGACAATCGTGAGAAGATTGAAAAAATCTTGACTTTT
CGAATTCCTTATTATGTTGGTCCATTGGCGCGTGGCAATAGTCGTTTTGCATGGATGACTC
GGAAGTCTGAAGAAACAATTACCCCATGGAATTTTGAAGAAGTTGTCGATAAAGGTGCTT
CAGCTCAATCATTTATTGAACGCATGACAAACTTTGATAAAAATCTTCCAAATGAAAAAG
TACTACCAAAACATAGTTTGCTTTATGAGTATTTTACGGTTTATAACGAATTGACAAAGGT
CAAATATGTTACTGAAGGAATGCGAAAACCAGCATTTCTTTCAGGTGAACAGAAGAAAG
CCATTGTTGATTTACTCTTCAAAACAAATCGAAAAGTAACCGTTAAGCAATTAAAAGAAG
ATTATTTCAAAAAAATAGAATGTTTTGATAGTGTTGAAATTTCAGGAGTTGAAGATAGAT
TTAATGCTTCATTAGGTACCTACCATGATTTGCTAAAAATTATTAAAGATAAAGATTTTTT
GGATAATGAAGAAAATGAAGATATCTTAGAGGATATTGTTTTAACATTGACCTTATTTGA
AGATAGGGAGATGATTGAGGAAAGACTTAAAACATATGCTCACCTCTTTGATGATAAGGT
GATGAAACAGCTTAAACGTCGCCGTTATACTGGTTGGGGACGTTTGTCTCGAAAATTGAT
TAATGGTATTAGGGATAAGCAATCTGGCAAAACAATATTAGATTTTTTGAAATCAGATGG
TTTTGCCAATCGCAATTTTATGCAGCTGATCCATGATGATAGTTTGACATTTAAAGAAGAC
ATTCAAAAAGCACAAGTGTCTGGACAAGGCGATAGTTTACATGAACATATTGCAAATTTA
GCTGGTAGCCCTGCTATTAAAAAAGGTATTTTACAGACTGTAAAAGTTGTTGATGAATTG
GTCAAAGTAATGGGGCGGCATAAGCCAGAAAATATCGTTATTGAAATGGCACGTGAAAA
TGCGACAACTCAAAAGGGCCAGAAAAATTCGCGAGAGCGTATGAAACGAATCGAAGAAG
GTATCAAAGAATTAGGAAGTCAGATTCTTAAAGAGCATCCTGTTGAAAATACTCAATTGC
AAAATGAAAAGCTCTATCTCTATTATCTCCAAAATGGAAGAGACATGTATGTGGACCAAG
AATTAGATATTAATCGTTTAAGTGATTATGATGTCGATCACATTGTTCCACAAAGTTTCCT
TAAAGACGATTCAATAGACAATAAGGTCTTAACGCGTTCTGATAAAAATCGTGGTAAATC
GGATAACGTTCCAAGTGAAGAAGTAGTCAAAAAGATGAAAAACTATTGGAGACAACTTC
TAAACGCCAAGTTAATCACTCAACGTAAGTTTGATAATTTAACGAAAGCTGAACGTGGAG
GTTTGAGTGAACTTGATAAAGCTGGTTTTATCAAACGCCAATTGGTTGAAACTCGCCAAA
TCACTAAGCATGTGGCACAAATTTTGGATAGTCGCATGAATACTAAATACGATGAAAATG
ATAAACTTATTCGAGAGGTTAAAGTGATTACCTTAAAATCTAAATTAGTTTCTGACTTCCG
AAAAGATTTCCAATTCTATAAAGTACGTGAGATTAACAATTACCATCATGCCCATGATGC
GTATCTAAATGCCGTCGTTGGAACTGCTTTGATTAAGAAATATCCAAAACTTGAATCGGA
GTTTGTCTATGGTGATTATAAAGTTTATGATGTTCGTAAAATGATTGCTAAGTCTGAGCAA
GAAATAGGCAAAGCAACCGCAAAATATTTCTTTTACTCTAATATCATGAACTTCTTCAAA
ACAGAAATTACACTTGCAAATGGAGAGATTCGCAAACGCCCTCTAATCGAAACTAATGG
GGAAACTGGAGAAATTGTCTGGGATAAAGGGCGAGATTTTGCCACAGTGCGCAAAGTAT
TGTCCATGCCCCAAGTCAATATTGTCAAGAAAACAGAAGTACAGACAGGCGGATTCTCCA
AGGAGTCAATTTTACCAAAAAGAAATTCGGACAAGCTTATTGCTCGTAAAAAAGACTGG
GATCCAAAAAAATATGGTGGTTTTGATAGTCCAACGGTAGCTTATTCAGTCCTAGTGGTT
GCTAAGGTGGAAAAAGGGAAATCGAAGAAGTTAAAATCCGTTAAAGAGTTACTAGGGAT
CACAATTATGGAAAGAAGTTCCTTTGAAAAAAATCCGATTGACTTTTTAGAAGCTAAAGG
ATATAAGGAAGTTAAAAAAGACTTAATCATTAAACTACCTAAATATAGTCTTTTTGAGTT
AGAAAACGGTCGTAAACGGATGCTGGCTAGTGCCGGAGAATTACAAAAAGGAAATGAGC
TGGCTCTGCCAAGCAAATATGTGAATTTTTTATATTTAGCTAGTCATTATGAAAAGTTGAA
GGGTAGTCCAGAAGATAACGAACAAAAACAATTGTTTGTGGAGCAGCATAAGCATTATTT
AGATGAGATTATTGAGCAAATCAGTGAATTTTCTAAGCGTGTTATTTTAGCAGATGCCAA
TTTAGATAAAGTTCTTAGTGCATATAACAAACATAGAGACAAACCAATACGTGAACAAGC
AGAAAATATTATTCATTTATTTACGTTGACGAATCTTGGAGCTCCCGCTGCTTTTAAATAT
TTTGATACAACAATTGATCGTAAACGATATACGTCTACAAAAGAAGTTTTAGATGCCACT
CTTATCCATCAATCCATCACTGGTCTTTATGAAACACGCATTGATTTGAGTCAGCTAGGAG
GTGACTGA
Sequence for S. pyogenes Cas9 (including NLS and C-terminal Flag-Tag), used for in
vivo experiments in HaCat and MCF7, note that the sequence is Codon optimized for
expression in these cell lines; SEQ ID NO: 18:
ATGGACAAGAAGTACAGCATCGGCCTGGACATCGGCACCAACTCTGTGGGCTGGGCCGT
GATCACCGACGAGTACAAGGTGCCCAGCAAGAAATTCAAGGTGCTGGGCAACACCGACC
GGCACAGCATCAAGAAGAACCTGATCGGAGCCCTGCTGTTCGACAGCGGCGAAACAGCC
GAGGCCACCGCCCTGAAGAGAACCGCCAGAAGAAGATACACCAGACGGAAGAACCGGA
TCTGCTATCTGCAAGAGATCTTCAGCAACGAGATGGCCAAGGTGGACGACAGCTTCTTCC
ACAGACTGGAAGAGTCCTTCCTGGTGGAAGAGGATAAGAAGCACGAGCGGCACCCCATC
TTCGGCAACATCGTGGACGAGGTGGCCTACCACGAGAAGTACCCCACCATCTACCACCTG
AGAAAGAAACTGGTGGACAGCACCGACAAGGCCGACCTGCGGCTGATCTATCTGGCCCT
GGCCCACATGATCAAGTTCCGGGGCCACTTCCTGATCGAGGGCGACCTGAACCCCGACAA
CAGCGACGTGGACAAGCTGTTCATCCAGCTGGTGCAGACCTACAACCAGCTGTTCGAGGA
AAACCCCATCAACGCCAGCGGCGTGGACGCCAAGGCCATCCTGTCTGCCAGACTGAGCA
AGAGCAGACGGCTGGAAAATCTGATCGCCCAGCTGCCCGGCGAGAAGAAGAATGGCCTG
TTCGGAAACCTGATTGCCCTGAGCCTGGGCCTGACCCCCAACTTCAAGAGCAACTTCGAC
CTGGCCGAGGATGCCAAACTGCAGCTGAGCAAGGACACCTACGACGACGACCTGGACAA
CCTGCTGGCCCAGATCGGCGACCAGTACGCCGACCTGTTTCTGGCCGCCAAGAACCTGTC
CGACGCCATCCTGCTGAGCGACATCCTGAGAGTGAACACCGAGATCACCAAGGCCCCCCT
GAGCGCCTCTATGATCAAGAGATACGACGAGCACCACCAGGACCTGACCCTGCTGAAAG
CTCTCGTGCGGCAGCAGCTGCCTGAGAAGTACAAAGAGATTTTCTTCGACCAGAGCAAGA
ACGGCTACGCCGGCTACATTGACGGCGGAGCCAGCCAGGAAGAGTTCTACAAGTTCATC
AAGCCCATCCTGGAAAAGATGGACGGCACCGAGGAACTGCTCGTGAAGCTGAACAGAGA
GGACCTGCTGCGGAAGCAGCGGACCTTCGACAACGGCAGCATCCCCCACCAGATCCACCT
GGGAGAGCTGCACGCCATTCTGCGGCGGCAGGAAGATTTTTACCCATTCCTGAAGGACAA
CCGGGAAAAGATCGAGAAGATCCTGACCTTCCGCATCCCCTACTACGTGGGCCCTCTGGC
CAGGGGAAACAGCAGATTCGCCTGGATGACCAGAAAGAGCGAGGAAACCATCACCCCCT
GGAACTTCGAGGAAGTGGTGGACAAGGGCGCTTCCGCCCAGAGCTTCATCGAGCGGATG
ACCAACTTCGATAAGAACCTGCCCAACGAGAAGGTGCTGCCCAAGCACAGCCTGCTGTAC
GAGTACTTCACCGTGTATAACGAGCTGACCAAAGTGAAATACGTGACCGAGGGAATGAG
AAAGCCCGCCTTCCTGAGCGGCGAGCAGAAAAAGGCCATCGTGGACCTGCTGTTCAAGA
CCAACCGGAAAGTGACCGTGAAGCAGCTGAAAGAGGACTACTTCAAGAAAATCGAGTGC
TTCGACTCCGTGGAAATCTCCGGCGTGGAAGATCGGTTCAACGCCTCCCTGGGCACATAC
CACGATCTGCTGAAAATTATCAAGGACAAGGACTTCCTGGACAATGAGGAAAACGAGGA
CATTCTGGAAGATATCGTGCTGACCCTGACACTGTTTGAGGACAGAGAGATGATCGAGGA
ACGGCTGAAAACCTATGCCCACCTGTTCGACGACAAAGTGATGAAGCAGCTGAAGCGGC
GGAGATACACCGGCTGGGGCAGGCTGAGCCGGAAGCTGATCAACGGCATCCGGGACAAG
CAGTCCGGCAAGACAATCCTGGATTTCCTGAAGTCCGACGGCTTCGCCAACAGAAACTTC
ATGCAGCTGATCCACGACGACAGCCTGACCTTTAAAGAGGACATCCAGAAAGCCCAGGT
GTCCGGCCAGGGCGATAGCCTGCACGAGCACATTGCCAATCTGGCCGGCAGCCCCGCCAT
TAAGAAGGGCATCCTGCAGACAGTGAAGGTGGTGGACGAGCTCGTGAAAGTGATGGGCC
GGCACAAGCCCGAGAACATCGTGATCGAAATGGCCAGAGAGAACCAGACCACCCAGAAG
GGACAGAAGAACAGCCGCGAGAGAATGAAGCGGATCGAAGAGGGCATCAAAGAGCTGG
GCAGCCAGATCCTGAAAGAACACCCCGTGGAAAACACCCAGCTGCAGAACGAGAAGCTG
TACCTGTACTACCTGCAGAATGGGCGGGATATGTACGTGGACCAGGAACTGGACATCAAC
CGGCTGTCCGACTACGATGTGGACCATATCGTGCCTCAGAGCTTTCTGAAGGACGACTCC
ATCGACAACAAGGTGCTGACCAGAAGCGACAAGAACCGGGGCAAGAGCGACAACGTGC
CCTCCGAAGAGGTCGTGAAGAAGATGAAGAACTACTGGCGGCAGCTGCTGAACGCCAAG
CTGATTACCCAGAGAAAGTTCGACAATCTGACCAAGGCCGAGAGAGGCGGCCTGAGCGA
ACTGGATAAGGCCGGCTTCATCAAGAGACAGCTGGTGGAAACCCGGCAGATCACAAAGC
ACGTGGCACAGATCCTGGACTCCCGGATGAACACTAAGTACGACGAGAATGACAAGCTG
ATCCGGGAAGTGAAAGTGATCACCCTGAAGTCCAAGCTGGTGTCCGATTTCCGGAAGGAT
TTCCAGTTTTACAAAGTGCGCGAGATCAACAACTACCACCACGCCCACGACGCCTACCTG
AACGCCGTCGTGGGAACCGCCCTGATCAAAAAGTACCCTAAGCTGGAAAGCGAGTTCGT
GTACGGCGACTACAAGGTGTACGACGTGCGGAAGATGATCGCCAAGAGCGAGCAGGAAA
TCGGCAAGGCTACCGCCAAGTACTTCTTCTACAGCAACATCATGAACTTTTTCAAGACCG
AGATTACCCTGGCCAACGGCGAGATCCGGAAGCGGCCTCTGATCGAGACAAACGGCGAA
ACCGGGGAGATCGTGTGGGATAAGGGCCGGGATTTTGCCACCGTGCGGAAAGTGCTGAG
CATGCCCCAAGTGAATATCGTGAAAAAGACCGAGGTGCAGACAGGCGGCTTCAGCAAAG
AGTCTATCCTGCCCAAGAGGAACAGCGATAAGCTGATCGCCAGAAAGAAGGACTGGGAC
CCTAAGAAGTACGGCGGCTTCGACAGCCCCACCGTGGCCTATTCTGTGCTGGTGGTGGCC
AAAGTGGAAAAGGGCAAGTCCAAGAAACTGAAGAGTGTGAAAGAGCTGCTGGGGATCA
CCATCATGGAAAGAAGCAGCTTCGAGAAGAATCCCATCGACTTTCTGGAAGCCAAGGGC
TACAAAGAAGTGAAAAAGGACCTGATCATCAAGCTGCCTAAGTACTCCCTGTTCGAGCTG
GAAAACGGCCGGAAGAGAATGCTGGCCTCTGCCGGCGAACTGCAGAAGGGAAACGAACT
GGCCCTGCCCTCCAAATATGTGAACTTCCTGTACCTGGCCAGCCACTATGAGAAGCTGAA
GGGCTCCCCCGAGGATAATGAGCAGAAACAGCTGTTTGTGGAACAGCACAAGCACTACC
TGGACGAGATCATCGAGCAGATCAGCGAGTTCTCCAAGAGAGTGATCCTGGCCGACGCT
AATCTGGACAAAGTGCTGTCCGCCTACAACAAGCACCGGGATAAGCCCATCAGAGAGCA
GGCCGAGAATATCATCCACCTGTTTACCCTGACCAATCTGGGAGCCCCTGCCGCCTTCAA
GTACTTTGACACCACCATCGACCGGAAGAGGTACACCAGCACCAAAGAGGTGCTGGACG
CCACCCTGATCCACCAGAGCATCACCGGCCTGTACGAGACACGGATCGACCTGTCTCAGC
TGGGAGGCGACAAGCGACCTGCCGCCACAAAGAAGGCTGGACAGGCTAAGAAGAAGAA
AGATTACAAAGACGATGACGATAAG
Sequence for S. pyogenes Cas9_R63A/Q768A (including NLS and C-terminal Flag-Tag), used
for in vivo experiments in HaCat and MCF7, note that the sequence is Codon optimized
for expression in these cell lines; SEQ ID NO: 19:
ATGGACAAGAAGTACAGCATCGGCCTGGACATCGGCACCAACTCTGTGGGCTGGGCCGT
GATCACCGACGAGTACAAGGTGCCCAGCAAGAAATTCAAGGTGCTGGGCAACACCGACC
GGCACAGCATCAAGAAGAACCTGATCGGAGCCCTGCTGTTCGACAGCGGCGAAACAGCC
GAGGCCACCGCCCTGAAGAGAACCGCCAGAAGAAGATACACCAGACGGAAGAACCGGA
TCTGCTATCTGCAAGAGATCTTCAGCAACGAGATGGCCAAGGTGGACGACAGCTTCTTCC
ACAGACTCGAGGAGTCCTTCCTGGTGGAAGAGGATAAGAAGCACGAGCGGCACCCCATC
TTCGGCAACATCGTGGACGAGGTGGCCTACCACGAGAAGTACCCCACCATCTACCACCTG
AGAAAGAAACTGGTGGACAGCACCGACAAGGCCGACCTGCGGCTGATCTATCTGGCCCT
GGCCCACATGATCAAGTTCCGGGGCCACTTCCTGATCGAGGGCGACCTGAACCCCGACAA
CAGCGACGTGGACAAGCTGTTCATCCAGCTGGTGCAGACCTACAACCAGCTGTTCGAGGA
AAACCCCATCAACGCCAGCGGCGTGGACGCCAAGGCCATCCTGTCTGCCAGACTGAGCA
AGAGCAGACGGCTGGAAAATCTGATCGCCCAGCTGCCCGGCGAGAAGAAGAATGGCCTG
TTCGGAAACCTGATTGCCCTGAGCCTGGGCCTGACCCCCAACTTCAAGAGCAACTTCGAC
CTGGCCGAGGATGCCAAACTGCAGCTGAGCAAGGACACCTACGACGACGACCTGGACAA
CCTGCTGGCCCAGATCGGCGACCAGTACGCCGACCTGTTTCTGGCCGCCAAGAACCTGTC
CGACGCCATCCTGCTGAGCGACATCCTGAGAGTGAACACCGAGATCACCAAGGCCCCCCT
GAGCGCCTCTATGATCAAGAGATACGACGAGCACCACCAGGACCTGACCCTGCTGAAAG
CTCTCGTGCGGCAGCAGCTGCCTGAGAAGTACAAAGAGATTTTCTTCGACCAGAGCAAGA
ACGGCTACGCCGGCTACATTGACGGCGGAGCCAGCCAGGAAGAGTTCTACAAGTTCATC
AAGCCCATCCTGGAAAAGATGGACGGCACCGAGGAACTGCTCGTGAAGCTGAACAGAGA
GGACCTGCTGCGGAAGCAGCGGACCTTCGACAACGGCAGCATCCCCCACCAGATCCACCT
GGGAGAGCTGCACGCCATTCTGCGGCGGCAGGAAGATTTTTACCCATTCCTGAAGGACAA
CCGGGAAAAGATCGAGAAGATCCTGACCTTCCGCATCCCCTACTACGTGGGCCCTCTGGC
CAGGGGAAACAGCAGATTCGCCTGGATGACCAGAAAGAGCGAGGAAACCATCACCCCCT
GGAACTTCGAGGAAGTGGTGGACAAGGGCGCTTCCGCCCAGAGCTTCATCGAGCGGATG
ACCAACTTCGATAAGAACCTGCCCAACGAGAAGGTGCTGCCCAAGCACAGCCTGCTGTAC
GAGTACTTCACCGTGTATAACGAGCTGACCAAAGTGAAATACGTGACCGAGGGAATGAG
AAAGCCCGCCTTCCTGAGCGGCGAGCAGAAAAAGGCCATCGTGGACCTGCTGTTCAAGA
CCAACCGGAAAGTGACCGTGAAGCAGCTGAAAGAGGACTACTTCAAGAAAATCGAGTGC
TTCGACTCCGTGGAAATCTCCGGCGTGGAAGATCGGTTCAACGCCTCCCTGGGCACATAC
CACGATCTGCTGAAAATTATCAAGGACAAGGACTTCCTGGACAATGAGGAAAACGAGGA
CATTCTGGAAGATATCGTGCTGACCCTGACACTGTTTGAGGACAGAGAGATGATCGAGGA
ACGGCTGAAAACCTATGCCCACCTGTTCGACGACAAAGTGATGAAGCAGCTGAAGCGGC
GGAGATACACCGGCTGGGGCAGGCTGAGCCGGAAGCTGATCAACGGCATCCGGGACAAG
CAGTCCGGCAAGACAATCCTGGATTTCCTGAAGTCCGACGGCTTCGCCAACAGAAACTTC
ATGCAGCTGATCCACGACGACAGCCTGACCTTTAAAGAGGACATCCAGAAAGCCCAGGT
GTCCGGCCAGGGCGATAGCCTGCACGAGCACATTGCCAATCTGGCCGGCAGCCCCGCCAT
TAAGAAGGGCATCCTGCAGACAGTGAAGGTGGTGGACGAGCTCGTGAAAGTGATGGGCC
GGCACAAGCCCGAGAACATCGTGATCGAAATGGCCAGAGAGAACGCCACCACCCAGAAG
GGACAGAAGAACAGCCGCGAGAGAATGAAGCGGATCGAAGAGGGCATCAAAGAGCTGG
GCAGCCAGATCCTGAAAGAACACCCCGTGGAAAACACCCAGCTGCAGAACGAGAAGCTG
TACCTGTACTACCTGCAGAATGGGCGGGATATGTACGTGGACCAGGAACTGGACATCAAC
CGGCTGTCCGACTACGATGTGGACCATATCGTGCCTCAGAGCTTTCTGAAGGACGACTCC
ATCGACAACAAGGTGCTGACCAGAAGCGACAAGAACCGGGGCAAGAGCGACAACGTGC
CCTCCGAAGAGGTCGTGAAGAAGATGAAGAACTACTGGCGCCAGCTGCTGAACGCCAAG
CTGATTACCCAGAGAAAGTTCGACAATCTGACCAAGGCCGAGAGAGGCGGCCTGAGCGA
ACTGGATAAGGCCGGCTTCATCAAGAGACAGCTGGTGGAAACCCGGCAGATCACAAAGC
ACGTGGCACAGATCCTGGACTCCCGGATGAACACTAAGTACGACGAGAATGACAAGCTG
ATCCGGGAAGTGAAAGTGATCACCCTGAAGTCCAAGCTGGTGTCCGATTTCCGGAAGGAT
TTCCAGTTTTACAAAGTGCGCGAGATCAACAACTACCACCACGCCCACGACGCCTACCTG
AACGCCGTCGTGGGAACCGCCCTGATCAAAAAGTACCCTAAGCTGGAAAGCGAGTTCGT
GTACGGCGACTACAAGGTGTACGACGTGCGGAAGATGATCGCCAAGAGCGAGCAGGAAA
TCGGCAAGGCTACCGCCAAGTACTTCTTCTACAGCAACATCATGAACTTTTTCAAGACCG
AGATTACCCTGGCCAACGGCGAGATCCGGAAGCGGCCTCTGATCGAGACAAACGGCGAA
ACCGGGGAGATCGTGTGGGATAAGGGCCGGGATTTTGCCACCGTGCGGAAAGTGCTGAG
CATGCCCCAAGTGAATATCGTGAAAAAGACCGAGGTGCAGACAGGCGGCTTCAGCAAAG
AGTCTATCCTGCCCAAGAGGAACAGCGATAAGCTGATCGCCAGAAAGAAGGACTGGGAC
CCTAAGAAGTACGGCGGCTTCGACAGCCCCACCGTGGCCTATTCTGTGCTGGTGGTGGCC
AAAGTGGAAAAGGGCAAGTCCAAGAAACTGAAGAGTGTGAAAGAGCTGCTGGGGATCA
CCATCATGGAAAGAAGCAGCTTCGAGAAGAATCCCATCGACTTTCTGGAAGCCAAGGGC
TACAAAGAAGTGAAAAAGGACCTGATCATCAAGCTGCCTAAGTACTCCCTGTTCGAGCTG
GAAAACGGCCGGAAGAGAATGCTGGCCTCTGCCGGCGAACTGCAGAAGGGAAACGAACT
GGCCCTGCCCTCCAAATATGTGAACTTCCTGTACCTGGCCAGCCACTATGAGAAGCTGAA
GGGCTCCCCCGAGGATAATGAGCAGAAACAGCTGTTTGTGGAACAGCACAAGCACTACC
TGGACGAGATCATCGAGCAGATCAGCGAGTTCTCCAAGAGAGTGATCCTGGCCGACGCT
AATCTGGACAAAGTGCTGTCCGCCTACAACAAGCACCGGGATAAGCCCATCAGAGAGCA
GGCCGAGAATATCATCCACCTGTTTACCCTGACCAATCTGGGAGCCCCTGCCGCCTTCAA
GTACTTTGACACCACCATCGACCGGAAGAGGTACACCAGCACCAAAGAGGTGCTGGACG
CCACCCTGATCCACCAGAGCATCACCGGCCTGTACGAGACACGGATCGACCTGTCTCAGC
TGGGAGGCGACAAGCGACCTGCCGCCACAAAGAAGGCTGGACAGGCTAAGAAGAAGAA
AGATTACAAAGACGATGACGATAAG
Sequence for S. pyogenes Cas9_R66A/Q768A (including NLS and C-terminal Flag-Tag),
used for in vivo experiments in HaCat and MCF7, note that the sequence is Codon
optimized for expression in these cell lines; SEQ ID NO: 20:
ATGGACAAGAAGTA CAGCATCGGCCTGGACATCGGCAC CAA CTCTGTGGGCTGGGCCGT
GATCACCGACGAGTACAAGGTGCCCAGCAAGAAATTCAAGGTGCTGGGCAACACCGACC
GGCACAGCATCAAGAAGAACCTGATCGGAGCCCTGCTGTTCGACAGCGGCGAAACAGCC
GAGGCCACCCGGCTGAAGGCAACCGCCAGAAGAAGATACACCAGACGGAAGAACCGGA
TCTGCTATCTGCAAGAGATCTTCAGCAACGAGATGGCCAAGGTGGACGACAGCTTCTTCC
ACAGACTCGAGGAGTCCTTCCTGGTGGAAGAGGATAAGAAGCACGAGCGGCACCCCATC
TTCGGCAACATCGTGGACGAGGTGGCCTACCACGAGAAGTACCCCACCATCTACCACCTG
AGAAAGAAACTGGTGGACAGCACCGACAAGGCCGACCTGCGGCTGATCTATCTGGCCCT
GGCCCACATGATCAAGTTCCGGGGCCACTTCCTGATCGAGGGCGACCTGAACCCCGACAA
CAGCGACGTGGACAAGCTGTTCATCCAGCTGGTGCAGACCTACAACCAGCTGTTCGAGGA
AAACCCCATCAACGCCAGCGGCGTGGACGCCAAGGCCATCCTGTCTGCCAGACTGAGCA
AGAGCAGACGGCTGGAAAATCTGATCGCCCAGCTGCCCGGCGAGAAGAAGAATGGCCTG
TTCGGAAACCTGATTGCCCTGAGCCTGGGCCTGACCCCCAACTTCAAGAGCAACTTCGAC
CTGGCCGAGGATGCCAAACTGCAGCTGAGCAAGGACACCTACGACGACGACCTGGACAA
CCTGCTGGCCCAGATCGGCGACCAGTACGCCGACCTGTTTCTGGCCGCCAAGAACCTGTC
CGACGCCATCCTGCTGAGCGACATCCTGAGAGTGAACACCGAGATCACCAAGGCCCCCCT
GAGCGCCTCTATGATCAAGAGATACGACGAGCACCACCAGGACCTGACCCTGCTGAAAG
CTCTCGTGCGGCAGCAGCTGCCTGAGAAGTACAAAGAGATTTTCTTCGACCAGAGCAAGA
ACGGCTACGCCGGCTACATTGACGGCGGAGCCAGCCAGGAAGAGTTCTACAAGTTCATC
AAGCCCATCCTGGAAAAGATGGACGGCACCGAGGAACTGCTCGTGAAGCTGAACAGAGA
GGACCTGCTGCGGAAGCAGCGGACCTTCGACAACGGCAGCATCCCCCACCAGATCCACCT
GGGAGAGCTGCACGCCATTCTGCGGCGGCAGGAAGATTTTTACCCATTCCTGAAGGACAA
CCGGGAAAAGATCGAGAAGATCCTGACCTTCCGCATCCCCTACTACGTGGGCCCTCTGGC
CAGGGGAAACAGCAGATTCGCCTGGATGACCAGAAAGAGCGAGGAAACCATCACCCCCT
GGAACTTCGAGGAAGTGGTGGACAAGGGCGCTTCCGCCCAGAGCTTCATCGAGCGGATG
ACCAACTTCGATAAGAACCTGCCCAACGAGAAGGTGCTGCCCAAGCACAGCCTGCTGTAC
GAGTACTTCACCGTGTATAACGAGCTGACCAAAGTGAAATACGTGACCGAGGGAATGAG
AAAGCCCGCCTTCCTGAGCGGCGAGCAGAAAAAGGCCATCGTGGACCTGCTGTTCAAGA
CCAACCGGAAAGTGACCGTGAAGCAGCTGAAAGAGGACTACTTCAAGAAAATCGAGTGC
TTCGACTCCGTGGAAATCTCCGGCGTGGAAGATCGGTTCAACGCCTCCCTGGGCACATAC
CACGATCTGCTGAAAATTATCAAGGACAAGGACTTCCTGGACAATGAGGAAAACGAGGA
CATTCTGGAAGATATCGTGCTGACCCTGACACTGTTTGAGGACAGAGAGATGATCGAGGA
ACGGCTGAAAACCTATGCCCACCTGTTCGACGACAAAGTGATGAAGCAGCTGAAGCGGC
GGAGATACACCGGCTGGGGCAGGCTGAGCCGGAAGCTGATCAACGGCATCCGGGACAAG
CAGTCCGGCAAGACAATCCTGGATTTCCTGAAGTCCGACGGCTTCGCCAACAGAAACTTC
ATGCAGCTGATCCACGACGACAGCCTGACCTTTAAAGAGGACATCCAGAAAGCCCAGGT
GTCCGGCCAGGGCGATAGCCTGCACGAGCACATTGCCAATCTGGCCGGCAGCCCCGCCAT
TAAGAAGGGCATCCTGCAGACAGTGAAGGTGGTGGACGAGCTCGTGAAAGTGATGGGCC
GGCACAAGCCCGAGAACATCGTGATCGAAATGGCCAGAGAGAACGCCACCACCCAGAAG
GGACAGAAGAACAGCCGCGAGAGAATGAAGCGGATCGAAGAGGGCATCAAAGAGCTGG
GCAGCCAGATCCTGAAAGAACACCCCGTGGAAAACACCCAGCTGCAGAACGAGAAGCTG
TACCTGTACTACCTGCAGAATGGGCGGGATATGTACGTGGACCAGGAACTGGACATCAAC
CGGCTGTCCGACTACGATGTGGACCATATCGTGCCTCAGAGCTTTCTGAAGGACGACTCC
ATCGACAACAAGGTGCTGACCAGAAGCGACAAGAACCGGGGCAAGAGCGACAACGTGC
CCTCCGAAGAGGTCGTGAAGAAGATGAAGAACTACTGGCGCCAGCTGCTGAACGCCAAG
CTGATTACCCAGAGAAAGTTCGACAATCTGACCAAGGCCGAGAGAGGCGGCCTGAGCGA
ACTGGATAAGGCCGGCTTCATCAAGAGACAGCTGGTGGAAACCCGGCAGATCACAAAGC
ACGTGGCACAGATCCTGGACTCCCGGATGAACACTAAGTACGACGAGAATGACAAGCTG
ATCCGGGAAGTGAAAGTGATCACCCTGAAGTCCAAGCTGGTGTCCGATTTCCGGAAGGAT
TTCCAGTTTTACAAAGTGCGCGAGATCAACAACTACCACCACGCCCACGACGCCTACCTG
AACGCCGTCGTGGGAACCGCCCTGATCAAAAAGTACCCTAAGCTGGAAAGCGAGTTCGT
GTACGGCGACTACAAGGTGTACGACGTGCGGAAGATGATCGCCAAGAGCGAGCAGGAAA
TCGGCAAGGCTACCGCCAAGTACTTCTTCTACAGCAACATCATGAACTTTTTCAAGACCG
AGATTACCCTGGCCAACGGCGAGATCCGGAAGCGGCCTCTGATCGAGACAAACGGCGAA
ACCGGGGAGATCGTGTGGGATAAGGGCCGGGATTTTGCCACCGTGCGGAAAGTGCTGAG
CATGCCCCAAGTGAATATCGTGAAAAAGACCGAGGTGCAGACAGGCGGCTTCAGCAAAG
AGTCTATCCTGCCCAAGAGGAACAGCGATAAGCTGATCGCCAGAAAGAAGGACTGGGAC
CCTAAGAAGTACGGCGGCTTCGACAGCCCCACCGTGGCCTATTCTGTGCTGGTGGTGGCC
AAAGTGGAAAAGGGCAAGTCCAAGAAACTGAAGAGTGTGAAAGAGCTGCTGGGGATCA
CCATCATGGAAAGAAGCAGCTTCGAGAAGAATCCCATCGACTTTCTGGAAGCCAAGGGC
TACAAAGAAGTGAAAAAGGACCTGATCATCAAGCTGCCTAAGTACTCCCTGTTCGAGCTG
GAAAACGGCCGGAAGAGAATGCTGGCCTCTGCCGGCGAACTGCAGAAGGGAAACGAACT
GGCCCTGCCCTCCAAATATGTGAACTTCCTGTACCTGGCCAGCCACTATGAGAAGCTGAA
GGGCTCCCCCGAGGATAATGAGCAGAAACAGCTGTTTGTGGAACAGCACAAGCACTACC
TGGACGAGATCATCGAGCAGATCAGCGAGTTCTCCAAGAGAGTGATCCTGGCCGACGCT
AATCTGGACAAAGTGCTGTCCGCCTACAACAAGCACCGGGATAAGCCCATCAGAGAGCA
GGCCGAGAATATCATCCACCTGTTTACCCTGACCAATCTGGGAGCCCCTGCCGCCTTCAA
GTACTTTGACACCACCATCGACCGGAAGAGGTACACCAGCACCAAAGAGGTGCTGGACG
CCACCCTGATCCACCAGAGCATCACCGGCCTGTACGAGACACGGATCGACCTGTCTCAGC
TGGGAGGCGACAAGCGACCTGCCGCCACAAAGAAGGCTGGACAGGCTAAGAAGAAGAA
AGATTACAAAGACGATGACGATAAG
tracrRNA for in vitro experiments; SEQ ID NO: 21:
AAAACAGCAUAGCAAGUUAAAAUAAGGCUAGUCCGUUAUCAACUUGAAAAAGUGGCA
CCGAGAGUCGGUGCUUUUUU
crRNA for in vitro experiments targeting speM protospacer; SEQ ID NO: 22:
AUAACUCAAUUUGUAAAAAAGUUUUAGAGCUAUGCUGUUUUG
sgRNA for in vivo E. coli assays targeting 5′ CDS of DsRed; SEQ ID NO: 23:
ugcaccuugaagcgcaugaaGUUUUAGAGCUAGAAAUAGCAAGUUAAAAUAAGGCUAGUCCGUU
AUCAACUUGAAAAAGUGGCACCGAGUCGGUGC
sgRNA for in vivo E. coli assays targeting 5′ CDS of DsRed, mismatch at position 1 (counting
from PAM); SEQ ID NO: 24:
ugcaccuugaagcgcaugaUGUUUUAGAGCUAGAAAUAGCAAGUUAAAAUAAGGCUAGUCCGUU
AUCAACUUGAAAAAGUGGCACCGAGUCGGUGC
sgRNA for in vivo E. coli assays targeting 5′ CDS of DsRed, mismatch at position 2 (counting
from PAM); SEQ ID NO: 25:
ugcaccuugaagcgcaugUaGUUUUAGAGCUAGAAAUAGCAAGUUAAAAUAAGGCUAGUCCGUU
AUCAACUUGAAAAAGUGGCACCGAGUCGGUGC
sgRNA for in vivo E. coli assays targeting 5′ CDS of DsRed, mismatch at position 3 (counting
from PAM); SEQ ID NO: 26:
ugcaccuugaagcgcauCaaGUUUUAGAGCUAGAAAUAGCAAGUUAAAAUAAGGCUAGUCCGUU
AUCAACUUGAAAAAGUGGCACCGAGUCGGUGC
sgRNA for in vivo E. coli assays targeting 5′ CDS of DsRed, mismatch at position 4 (counting
from PAM); SEQ ID NO: 27:
ugcaccuugaagcgcaAgaaGUUUUAGAGCUAGAAAUAGCAAGUUAAAAUAAGGCUAGU
CCGUUAUCAACUUGAAAAAGUGGCACCGAGUCGGUGC
sgRNA for in vivo E. coli assays targeting 5′ CDS of DsRed, mismatch at position 5 (counting
from PAM); SEQ ID NO: 28:
ugcaccuugaagcgcUugaaGUUUUAGAGCUAGAAAUAGCAAGUUAAAAUAAGGCUAGU
CCGUUAUCAACUUGAAAAAGUGGCACCGAGUCGGUGC
sgRNA for in vivo E. coli assays targeting 5′ CDS of DsRed, mismatch at position 6 (counting
from PAM); SEQ ID NO: 29:
ugcaccuugaagcgGaugaaGUUUUAGAGCUAGAAAUAGCAAGUUAAAAUAAGGCUAGU
CCGUUAUCAACUUGAAAAAGUGGCACCGAGUCGGUGC
sgRNA for in vivo E. coli assays targeting 5′ CDS of DsRed, mismatch at position 7 (counting
from PAM); SEQ ID NO: 30:
ugcaccuugaagcCcaugaaGUUUUAGAGCUAGAAAUAGCAAGUUAAAAUAAGGCUAGU
CCGUUAUCAACUUGAAAAAGUGGCACCGAGUCGGUGC
sgRNA for in vivo E. coli assays targeting 5′ CDS of DsRed, mismatch at position 8 (counting
from PAM); SEQ ID NO: 31:
ugcaccuugaagGgcaugaaGUUUUAGAGCUAGAAAUAGCAAGUUAAAAUAAGGCUAGU
CCGUUAUCAACUUGAAAAAGUGGCACCGAGUCGGUGC
sgRNA for in vivo E. coli assays targeting 5′ CDS of DsRed, mismatch at position 9 (counting
from PAM); SEQ ID NO: 32:
ugcaccuugaaCcgcaugaaGUUUUAGAGCUAGAAAUAGCAAGUUAAAAUAAGGCUAGU
CCGUUAUCAACUUGAAAAAGUGGCACCGAGUCGGUGC
sgRNA for in vivo E. coli assays targeting 5′ CDS of DsRed, mismatch at position 10 (counting
from PAM); SEQ ID NO: 33:
ugcaccuugaUgcgcaugaaGUUUUAGAGCUAGAAAUAGCAAGUUAAAAUAAGGCUAGU
CCGUUAUCAACUUGAAAAAGUGGCACCGAGUCGGUGC
sgRNA for in vivo E. coli assays targeting 5′ CDS of DsRed, mismatch at position 11 (counting
from PAM); SEQ ID NO: 34:
ugcaccuugUagcgcaugaaGUUUUAGAGCUAGAAAUAGCAAGUUAAAAUAAGGCUAGU
CCGUUAUCAACUUGAAAAAGUGGCACCGAGUCGGUGC
sgRNA for in vivo E. coli assays targeting 5′ CDS of DsRed, mismatch at position 12 (counting
from PAM); SEQ ID NO: 35:
ugcaccuuCaagcgcaugaaGUUUUAGAGCUAGAAAUAGCAAGUUAAAAUAAGGCUAGU
CCGUUAUCAACUUGAAAAAGUGGCACCGAGUCGGUGC
sgRNA for in vivo E. coli assays targeting 5′ CDS of DsRed, mismatch at position 13 (counting
from PAM); SEQ ID NO: 36:
ugcaccuAgaagcgcaugaaGUUUUAGAGCUAGAAAUAGCAAGUUAAAAUAAGGCUAGU
CCGUUAUCAACUUGAAAAAGUGGCACCGAGUCGGUGC
sgRNA for in vivo E. coli assays targeting 5′ CDS of DsRed, mismatch at position 14 (counting
from PAM); SEQ ID NO: 37:
ugcaccAugaagcgcaugaaGUUUUAGAGCUAGAAAUAGCAAGUUAAAAUAAGGCUAGU
CCGUUAUCAACUUGAAAAAGUGGCACCGAGUCGGUGC
sgRNA for in vivo E. coli assays targeting 5′ CDS of DsRed, mismatch at position 15 (counting
from PAM); SEQ ID NO: 38:
ugcacGuugaagcgcaugaaGUUUUAGAGCUAGAAAUAGCAAGUUAAAAUAAGGCUAGU
CCGUUAUCAACUUGAAAAAGUGGCACCGAGUCGGUGC
sgRNA for in vivo E. coli assays targeting 5′ CDS of DsRed, mismatch at position 16 (counting
from PAM); SEQ ID NO: 39:
ugcaGcuugaagcgcaugaaGUUUUAGAGCUAGAAAUAGCAAGUUAAAAUAAGGCUAGU
CCGUUAUCAACUUGAAAAAGUGGCACCGAGUCGGUGC
sgRNA for in vivo E. coli assays targeting 5′ CDS of DsRed, mismatch at position 17 (counting
from PAM); SEQ ID NO: 40:
ugcUccuugaagcgcaugaaGUUUUAGAGCUAGAAAUAGCAAGUUAAAAUAAGGCUAGU
CCGUUAUCAACUUGAAAAAGUGGCACCGAGUCGGUGC
sgRNA for in vivo E. coli assays targeting 5′ CDS of DsRed, mismatch at position 18 (counting
from PAM); SEQ ID NO: 41:
ugGaccuugaagcgcaugaaGUUUUAGAGCUAGAAAUAGCAAGUUAAAAUAAGGCUAGU
CCGUUAUCAACUUGAAAAAGUGGCACCGAGUCGGUGC
sgRNA for in vivo E. coli assays targeting 5′ CDS of DsRed, mismatch at position 19 (counting
from PAM); SEQ ID NO: 42:
uCcaccuugaagcgcaugaaGUUUUAGAGCUAGAAAUAGCAAGUUAAAAUAAGGCUAGU
CCGUUAUCAACUUGAAAAAGUGGCACCGAGUCGGUGC
sgRNA for in vivo E. coli assays targeting 5′ CDS of DsRed, mismatch at position 20 (counting
from PAM); SEQ ID NO: 43:
AgcaccuugaagcgcaugaaGUUUUAGAGCUAGAAAUAGCAAGUUAAAAUAAGGCUAGU
CCGUUAUCAACUUGAAAAAGUGGCACCGAGUCGGUGC
speM protospacer used for in vitro experiments (PAM in bold); SEQ ID NO: 44:
TTATATGAACATAACTCAATTTGTAAAAAATGG
speM protospacer used for in vitro experiments, mismatch to crRNA at position 1 (counted
from PAM); SEQ ID NO: 45:
TTATATGAACATAACTCAATTTGTAAAAATTGG
speM protospacer used for in vitro experiments, mismatch to crRNA at position 2 (counted
from PAM); SEQ ID NO: 46:
TTATATGAACATAACTCAATTTGTAAAATATGG
speM protospacer used for in vitro experiments, mismatch to crRNA at position 3 (counted
from PAM); SEQ ID NO: 47:
TTATATGAACATAACTCAATTTGTAAATAATGG
speM protospacer used for in vitro experiments, mismatch to crRNA at position 4 (counted
from PAM); SEQ ID NO: 48:
TTATATGAACATAACTCAATTTGTAATAAATGG
speM protospacer used for in vitro experiments, mismatch to crRNA at position 5 (counted
from PAM); SEQ ID NO: 49:
TTATATGAACATAACTCAATTTGTATAAAATGG
speM protospacer used for in vitro experiments, mismatch to crRNA at position 6 (counted
from PAM); SEQ ID NO: 50:
TTATATGAACATAACTCAATTTGTTAAAAATGG
speM protospacer used for in vitro experiments, mismatch to crRNA at position 7 (counted
from PAM); SEQ ID NO: 51:
TTATATGAACATAACTCAATTTGAAAAAAATGG
speM protospacer used for in vitro experiments, mismatch to crRNA at position 8 (counted
from PAM); SEQ ID NO: 52:
TTATATGAACATAACTCAATTTCTAAAAAATGG
speM protospacer used for in vitro experiments, mismatch to crRNA at position 9 (counted
from PAM); SEQ ID NO: 53:
TTATATGAACATAACTCAATTAGTAAAAAATGG
speM protospacer used for in vitro experiments, mismatch to crRNA at position 10 (counted
from PAM); SEQ ID NO: 54:
TTATATGAACATAACTCAATATGTAAAAAATGG
speM protospacer used for in vitro experiments, mismatch to crRNA at position 11 (counted
from PAM); SEQ ID NO: 55:
TTATATGAACATAACTCAAATTGTAAAAAATGG
speM protospacer used for in vitro experiments, mismatch to crRNA at position 12 (counted
from PAM); SEQ ID NO: 56:
TTATATGAACATAACTCATTTTGTAAAAAATGG
speM protospacer used for in vitro experiments, mismatch to crRNA at position 13 (counted
from PAM); SEQ ID NO: 57:
TTATATGAACATAACTCTATTTGTAAAAAATGG
speM protospacer used for in vitro experiments, mismatch to crRNA at position 14 (counted
from PAM); SEQ ID NO: 58:
TTATATGAACATAACTGAATTTGTAAAAAATGG
speM protospacer used for in vitro experiments, mismatch to crRNA at position 15 (counted
from PAM); SEQ ID NO: 59:
TTATATGAACATAACACAATTTGTAAAAAATGG
speM protospacer used for in vitro experiments, mismatch to crRNA at position 16 (counted
from PAM); SEQ ID NO: 60:
TTATATGAACATAAGTCAATTTGTAAAAAATGG
speM protospacer used for in vitro experiments, mismatch to crRNA at position 17 (counted
from PAM); SEQ ID NO: 61:
TTATATGAACATATCTCAATTTGTAAAAAATGG
speM protospacer used for in vitro experiments, mismatch to crRNA at position 18 (counted
from PAM); SEQ ID NO: 62:
TTATATGAACATTACTCAATTTGTAAAAAATGG
speM protospacer used for in vitro experiments, mismatch to crRNA at position 19 (counted
from PAM); SEQ ID NO: 63:
TTATATGAACAAAACTCAATTTGTAAAAAATGG
speM protospacer used for in vitro experiments, mismatch to crRNA at position 20 (counted
from PAM); SEQ ID NO: 64:
TTATATGAACTTAACTCAATTTGTAAAAAATGG
DSRed target sequence used in in vivo E.coli experiements (PAM in bold); SEQ ID NO: 65:
TGCACCTTGAAGCGCATGAAGGG

s2RNA Used for In Vivo Assays for Detecting Cas9 Activity in HaCat and MCF7 Cell Lines:

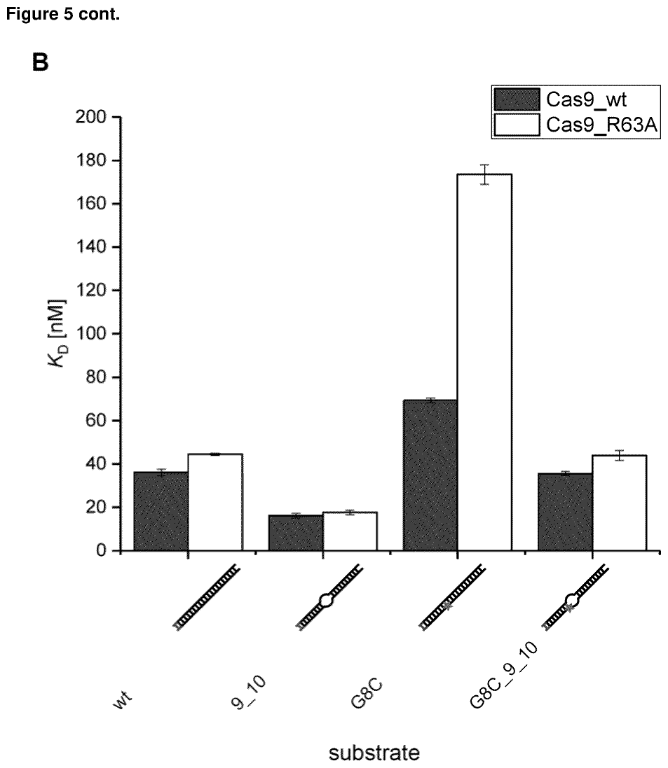

sgEpCAM-1; SEQ ID NO: 66:
GATCCTGACTGCGATGAGAG
sgEpCAM-2; SEQ ID NO: 67:
GATCACAACGCGTTATCAAC
sgEpCAM-3; SEQ ID NO: 68:
TAATGTTATCACTATTGATC
sgEpCAM-4; SEQ ID NO: 69:
CAAGCAGAAGCCCGAACGCG
FIG. 1, non-target strand, 5′-3′; SEQ ID NO: 70:
Tgaacataactcaatttgtaaaaaatcccgcag
FIG. 1, target strand; SEQ ID NO: 71:
Ctgcgccattttttacaaattgagttatgttca
FIG. 1, crRNA, 5′-3′; SEQ ID NO: 72:
AUAACUCAAUUUGUAAAAAAGUUUUAGA
FIG. 5, no bubble 1, 5′-3′; SEQ ID NO: 73:
TGAACATAACTCAATTTGTAAAAAATGGTACTC
FIG. 5, no bubble 2; SEQ ID NO: 74:
GAGTACCATTTTTTACAAATTGAGTTATGTTCA
FIG. 5,5 nt bubble 1, 5′-3′; SEQ ID NO: 75:
TGAACATAACTCAATTTGTATTTTTTGGTACTC
FIG. 5, 5 nt bubble 2; SEQ ID NO: 76:
GAGTACCATTTTTTACAAATTGAGTTATGTTCA
FIG. 5, 20 nt bubble 1, 5′-3′; SEQ ID NO: 77:
TGAACAGCCAGACCGGGTGCCCCCCTGGTACTC
FIG. 5, 20 nt bubble 2; SEQ ID NO: 78:
GAGTACCATTTTTTACAAATTGAGTTATGTTCA
FIG. 10, EpCAM-4, non-target strand, 5′-3′;
SEQ ID NO: 79:
CGCGGCAAGCAGAAGCCCGAACGCGAGGACCTG
FIG. 10, EpCAM-4, target strand; SEQ ID NO: 80:
CAGGTCCTCGCGTTCGGGCTTCTGCTTGCCGCG
FIG. 10, EpCAM-4, sgRNA, 5′-3′; SEQ ID NO: 81:
CAAGCAGAAGCCCGAACGCGGUUUUAGA
FIG. 10, EpCAM-1, non-target strand, 5′-3′;
SEQ ID NO: 82:
TTTATGATCCTGACTGCGATGAGAGCGGGCTCT
FIG. 10, EpCAM-1, target strand; SEQ ID NO: 83:
AGAGCCCGCTCTCATCGCAGTCAGGATCATAAA
FIG. 10, EpCAM-1, sgRNA; 5′-3′; SEQ ID NO: 84:
GAUCCUGACUGCGAUGAGAGGUUUUAGA
FIG. 11, VEGFA1 on-target; SEQ ID NO: 85:
GGGTGGGGGGAGTTTGCTCCTGG
FIG. 11, VEGFA1 off-target 6; SEQ ID NO: 86:
CGGGGGAGGGAGTTTGCTCCTGG
FIG. 11, VEGFA1 off-target 11; SEQ ID NO: 87:
GGGGAGGGGAAGTTTGCTCCTGG
FIG. 11, EMX1 on-target; SEQ ID NO: 88:
GAGTCCGAGCAGAAGAAGAAGGG
FIG. 11, EMX1 off-target 1; SEQ ID NO: 89:
GAGTTAGAGCAGAAGAAGAAAGG
FIG. 11, EMX1 off-target 52; SEQ ID NO: 90:
GAGTCCTAGCAGGAGAAGAAGAG
FIG. 11, EMX1 off-target 53; SEQ ID NO: 91:
GAGTCTAAGCAGGAGAAGAAGAG
FIG. 11, VEGFA3 on-target; SEQ ID NO: 92:
GGTGAGTGAGTGTGTGCGTGTGG
FIG. 11, VEGFA3 off-target 18; SEQ ID NO: 93:
TGTGGGTGAGTGTGTGCGTGAGG
FIG. 11, VEGFA3 off-target 4; SEQ ID NO: 94:
GCTGAGTGAGTGTATGCGTGTGG
FIG. 11, VEGFA3 off-target 2; SEQ ID NO: 95:
AGTGAGTGAGTGTGTGTGTGGGG

Primers for PCR Amplification of On- and Off-Target Sites:

OLEC10539 (forward); SEQ ID NO: 96:
TCCAGATGGCACATTGTCAG
OLEC10540 (reverse); SEQ ID NO: 97:
AGGGAGCAGGAAAGTGAGGT
OLEC10424 (forward); SEQ ID NO: 98
TCCAACGCCCTCAACCCCAC
OLEC10425 (reverse); SEQ ID NO: 99:
CACACTGTGGCCCCTGTGC
OLEC10426 (forward); SEQ ID NO: 100:
CAGCAGGACTGTGTGGCAC
OLEC10427 (reverse); SEQ ID NO: 101:
TCCTTCCAGCGATCCCATGG
OLEC10432 (forward); SEQ ID NO: 102:
GCCCATTCTTTTTGCAGTGGA
0LEC10433 (reverse); SEQ ID NO: 103:
GAGAGCAAGTTTGTTCCCCAGG
OLEC10434 (forward); SEQ ID NO: 104:
ATGGGAGCAGCTGGTCAGAG
OLEC10435 (reverse); SEQ ID NO: 105:
TGGTTGCCCACCCTAGTCAT
OLEC10436 (forward); SEQ ID NO: 106:
CAAGCTTTTCCTGACGCCCC
OLEC10437 (reverse); SEQ ID NO: 107:
TCCTGAAGACCTGTAATCTGACTCT
OLEC10438 (forward); SEQ ID NO: 108:
GTGACTTGTTCCTGGTTCTGCC
OLEC10439 (reverse); SEQ ID NO: 109:
AGCTGTCCTGTCTCATTGGCT
OLEC10440 (forward); SEQ ID NO: 110:
TGAAATCTCACCTGGGCGAGA
OLEC10441 (reverse); SEQ ID NO: 111:
TGCAGTCTGCCTTTTTGGGG
OLEC10442 (forward); SEQ ID NO: 112:
CTGGGTGAATGGAGCGAGCAG
0LEC10443 (reverse); SEQ ID NO: 113:
GCATTGGCGAGGAGGGAGCA
OLEC10444 (forward); SEQ ID NO: 114:
TCTGTCACCACACAGTTACCACC
OLEC10445 (reverse); SEQ ID NO: 115:
GTTGCCTGGGGATGGGGTAT
OLEC10446 (forward); SEQ ID NO: 116:
TCCTTTGAGGTTCATCCCCC
OLEC10447 (reverse); SEQ ID NO: 117:
CCAATCCAGGATGATTCCGC
OLEC10448 (forward); SEQ ID NO: 118:
GAGGGGGAAGTCACCGACAA
OLEC10449 (reverse); SEQ ID NO: 119:
TACCCGGGCCGTCTGTTAGA

1. A Streptococcus pyogenes Cas9 (SpCas9) protein comprising or consisting of
(i) a polypeptide with an amino acid sequence according to SEQ ID NO: 1 wherein the arginine at position 63 and the glutamine at position 768 are each replaced by alanine; or
(ii) a polypeptide with an amino acid sequence having at least 90% sequence identity to the amino acid sequence according to SEQ ID NO: 1, wherein the residue corresponding to the arginine at position 63 of SEQ ID NO: 1 and the residue corresponding to the glutamine at position 768 of SEQ ID NO: 1 are each replaced by alanine, and wherein said polypeptide has enhanced specificity compared to a polypeptide with the amino acid sequence according to SEQ ID NO: 1.

2. The SpCas9 protein according to claim 1 (i) having enhanced specificity compared to a polypeptide with the amino acid sequence according to SEQ ID NO: 1.

3. The SpCas9 protein according to claim 1 or 2 further comprising one or more mutations that decrease nuclease activity which are selected from:
(i) D10A or D10N, and/or
(ii) H840A, H840N or N840Y.

4. The SpCas9 protein according to any one of claims 1 to 3 further comprising one or more nuclear localization signal(s) and/or one or more tag(s).

5. A polynucleotide encoding the SpCas9 protein according to any one of claims 1 to 4.

6. The polynucleotide according to claim 5, wherein said polynucleotide is codon-optimized for expression in a eukaryotic cell.

7. A vector comprising the polynucleotide according to claim 5 or 6.

8. The vector according to claim 7, wherein said polynucleotide is operably linked to one or more transcription regulatory element(s).

9. A host cell comprising the SpCas9 protein according to any one of claims 1 to 4, and/or the polynucleotide according to claim 5 or 6, and/or the vector of claim 7 or 8.

10. A composition comprising a CRISPR complex, wherein the CRISPR complex comprises:
(i) a guide RNA and the SpCas9 protein according to any one of claims 1 to 4;
(ii) a guide RNA and the polynucleotide according to claim 5 or 6; or
(iii) a guide RNA and the vector according to claim 7 or 8.

11. The composition according to claim 10, wherein the guide RNA is a single guide RNA or a tracrRNA:crRNA duplex.

12. The composition according to claim 10 or 11 for use in treating a disease which is based on one or more mutation(s).

13. Method of treating a disease which is based on one or more mutation(s) comprising administering an effective amount of the composition according to claim 10 or 11 to a subject in need of such a treatment.

14. The composition for the use according to claim 12 or the method according to claim 13, wherein the disease is based on one mutation in the genome.

15. The composition for the use according to claim 12 or 14 or the method according to claim 13 or 14, wherein the disease is an inheritable disease.

16. The composition for the use according to any one of claims 12, 14 and 15, or the method according to any one of claims 13 to 15, wherein the disease is achondroplasia, alpha-1 antitrypsin deficiency, Alzheimer's disease, antiphospholipid syndrome, autism, autosomal dominant polycystic kidney disease, cancer (such as breast cancer, colon cancer, prostate cancer, or skin cancer), Charcot-Marie-Tooth, cri du chat, Crohn's disease, cystic fibrosis, dercum disease, down syndrome, duane syndrome, duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan Syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, Wilms-Tumour-Aniridia-Syndrom (WAGR) or Wilson disease.

17. Use of the composition according to claim 10 or 11 for genome engineering, provided that said use is not a method for treatment of the human or animal body by surgery or therapy, and provided that said use is not a method for modifying the germline genetic identity of human beings.

18. A method for genome engineering in a cell, wherein the method comprises any one of the following steps:
(i) contacting said cell with a guide RNA and the SpCas9 protein according to any one of claims 1 to 4; or
(ii) expressing in said cell a guide RNA and the SpCas9 protein according to any one of claims 1 to 4.

19. A pharmaceutical composition comprising
(i) a guide RNA and the SpCas9 protein according to any one of claims 1 to 4;
(ii) a guide RNA and the polynucleotide according to claim 5 or 6; or
(iii) a guide RNA and the vector according to claim 7 or 8.

20. The pharmaceutical composition according to claim 19 for use in treating a disease which is based on one or more mutation(s).

21. The pharmaceutical composition for the use according to claim 20, wherein the disease is based on one mutation in the genome.

22. The pharmaceutical composition for the use according to claim 20 or 21, wherein the disease is an inheritable disease.

23. The pharmaceutical composition according to any one of claims 19 to 22 for use in treating achondroplasia, alpha-1 antitrypsin deficiency, Alzheimer's disease, antiphospholipid syndrome, autism, autosomal dominant polycystic kidney disease, cancer (such as breast cancer, colon cancer, prostate cancer, or skin cancer), Charcot-Marie-Tooth, cri du chat, Crohn's disease, cystic fibrosis, dercum disease, down syndrome, duane syndrome, duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan Syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, Wilms-Tumour-Aniridia-Syndrom (WAGR) or Wilson disease.


SEQUENCE LISTING (78 sequences)

SEQ ID NO: 1 - protein, 1368 aa, Streptococcus pyogenes [sequence omitted]

SEQ ID NO: 2 - protein, 1368 aa, Artificial Sequence
  Wild type SpCas9 amino acid sequence wherein the arginine (R) at position 63 and the glutamine (Q) at position 768 are each replaced by alanine (A)
MDKKYSIGLDIGTNSVGWAVITDEYKVPSKKFKVLGNTDRHSIKKNLIGALLFDSGETAEATALKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENATTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 1368 aa, Artificial Sequence
  Wild type SpCas9 amino acid sequence wherein the arginine (R) at position 66 and the glutamine (Q) at position 768 are each replaced by alanine (A)
MDKKYSIGLDIGTNSVGWAVITDEYKVPSKKFKVLGNTDRHSIKKNLIGALLFDSGETAEATRLKATARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENATTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - DNA, 4179 bp, Artificial Sequence
  Sequence for S. pyogenes Cas9 (including N-terminal His-tag), recombinant expressed in E.coli, purified and used for in vitro experiments
atgggccatcatcatcatcatcatcatcatcatcacagcagcggccatatcgaaggtcgtcatagcgtcgacatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 5 - DNA, 4179 bp, Artificial Sequence
  Sequence for S. pyogenes Cas9_R63A (including N-terminal His-tag), recombinant expressed in E.coli, purified and used for in vitro experiments
atgggccatcatcatcatcatcatcatcatcatcacagcagcggccatatcgaaggtcgtcatagcgtcgacatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactgctctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatcttcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 6 - DNA, 4179 bp, Artificial Sequence
  Sequence for S. pyogenes Cas9_R66A (including N-terminal His-tag), recombinant expressed in E.coli, purified and used for in vitro experiments
atgggccatcatcatcatcatcatcatcatcatcacagcagcggccatatcgaaggtcgtcatagcgtcgacatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaagcgacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatcttcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 7 - DNA, 4179 bp, Artificial Sequence
  Sequence for S. pyogenes Cas9_R70A (including N-terminal His-tag), recombinant expressed in E.coli, purified and used for in vitro experiments
atgggccatcatcatcatcatcatcatcatcatcacagcagcggccatatcgaaggtcgtcatagcgtcgacatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtgcaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatcttcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 8 - DNA, 4179 bp, Artificial Sequence
  Sequence for S. pyogenes Cas9_R63A/Q768A (including N-terminal His-tag), recombinant expressed in E.coli, purified and used for in vitro experiments
atgggccatcatcatcatcatcatcatcatcatcacagcagcggccatatcgaaggtcgtcatagcgtcgacatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactgctctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatcttcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatgcgacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 9 - DNA, 4179 bp, Artificial Sequence
  Sequence for S. pyogenes Cas9 Q768A (including N-terminal His-tag), recombinant expressed in E.coli, purified and used for in vitro experiments
atgggccatcatcatcatcatcatcatcatcatcacagcagcggccatatcgaaggtcgtcatagcgtcgacatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatgcgacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 10 - DNA, 4179 bp, Artificial Sequence
  Sequence for S. pyogenes Cas9_R66A/Q768A (including N-terminal His-tag), recombinant expressed in E.coli, purified and used for in vitro experiments
atgggccatcatcatcatcatcatcatcatcatcacagcagcggccatatcgaaggtcgtcatagcgtcgacatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaagcgacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatcttcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatgcgacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 11 - DNA, 4167 bp, Artificial Sequence
  Sequence for S. pyogenes Cas9 (including N-terminal His-tag), used for in vivo experiments in E.coli, note that only the coding sequence for the His-tag differs, the CDS for Cas9 is the same as for the recombinant purified ones
atgaaacatcaccatcaccatcacaacactagtcatatcgaaggtcgtcatagcgtcgacatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 12 - DNA, 4167 bp, Artificial Sequence
  Sequence for S. pyogenes Cas9_R63A (including N-terminal His-tag), used for in vivo experiments in E.coli, note that only the coding sequence for the His-tag differs, the CDS for Cas9 is the same as for the recombinant purified ones
atgaaacatcaccatcaccatcacaacactagtcatatcgaaggtcgtcatagcgtcgacatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactgctctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatcttcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 13 - DNA, 4167 bp, Artificial Sequence
  Sequence for S. pyogenes Cas9_R66A (including N-terminal His-tag), used for in vivo experiments in E.coli, note that only the coding sequence for the His-tag differs, the CDS for Cas9 is the same as for the recombinant purified ones
atgaaacatcaccatcaccatcacaacactagtcatatcgaaggtcgtcatagcgtcgacatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaagcgacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatcttcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 14 - DNA, 4167 bp, Artificial Sequence
  Sequence for S. pyogenes Cas9_R70A (including N-terminal His-tag), used for in vivo experiments in E.coli, note that only the coding sequence for the His-tag differs, the CDS for Cas9 is the same as for the recombinant purified ones
atgaaacatcaccatcaccatcacaacactagtcatatcgaaggtcgtcatagcgtcgacatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtgcaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatcttcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 15 - DNA, 4167 bp, Artificial Sequence
  Sequence for S. pyogenes Cas9 Q768A (including N-terminal His-tag), used for in vivo experiments in E.coli, note that only the coding sequence for the His-tag differs, the CDS for Cas9 is the same as for the recombinant purified ones
atgaaacatcaccatcaccatcacaacactagtcatatcgaaggtcgtcatagcgtcgacatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatgcgacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 16 - DNA, 4167 bp, Artificial Sequence
  Sequence for S. pyogenes Cas9_R63A/Q768A (including N-terminal His-tag), used for in vivo experiments in E.coli, note that only the coding sequence for the His-tag differs, the CDS for Cas9 is the same as for the recombinant purified ones
atgaaacatcaccatcaccatcacaacactagtcatatcgaaggtcgtcatagcgtcgacatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactgctctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatcttcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatgcgacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 17 - DNA, 4167 bp, Artificial Sequence
  Sequence for S. pyogenes Cas9_R66A/Q768A (including N-terminal His-tag), used for in vivo experiments in E.coli, note that only the coding sequence for the His-tag differs, the CDS for Cas9 is the same as for the recombinant purified ones
atgaaacatcaccatcaccatcacaacactagtcatatcgaaggtcgtcatagcgtcgacatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaagcgacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatcttcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatgcgacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 18 - DNA, 4176 bp, Artificial Sequence
  Sequence for S. pyogenes Cas9 (including NLS and C-terminal Flag-Tag), used for in vivo experiments in HaCat and MCF7, note that the sequence is Codon optimized for expression in these cell lines
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccaccgccctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataag

SEQ ID NO: 19 - DNA, 4176 bp, Artificial Sequence
  Sequence for S. pyogenes Cas9_R63A/Q768A (including NLS and C-terminal Flag-Tag), used for in vivo experiments in HaCat and MCF7, note that the sequence is Codon optimized for expression in these cell lines
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccaccgccctgaagagaaccgccagaagaagatacaccagacggaagaaccggatctgctatctgcaagagatcttcagcaacgagatggccaaggtggacgacagcttcttccacagactcgaggagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaacgccaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcgccagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataag

SEQ ID NO: 20 - DNA, 4176 bp, Artificial Sequence
  Sequence for S. pyogenes Cas9_R66A/Q768A (including NLS and C-terminal Flag-Tag), used for in vivo experiments in HaCat and MCF7, note that the sequence is Codon optimized for expression in these cell lines
atggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaaggcaaccgccagaagaagatacaccagacggaagaaccggatctgctatctgcaagagatcttcagcaacgagatggccaaggtggacgacagcttcttccacagactcgaggagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaacgccaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcgccagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataag

SEQ ID NO: 21 - RNA, 77 nt, Artificial Sequence
  tracrRNA for in vitro experiments
aaaacagcauagcaaguuaaaauaaggcuaguccguuaucaacuugaaaaaguggcaccgagagucggugcuuuuuu

SEQ ID NO: 22 - RNA, 42 nt, Artificial Sequence
  crRNA for in vitro experiments targeting speM protospacer
auaacucaauuuguaaaaaaguuuuagagcuaugcuguuuug

SEQ ID NO: 23 - RNA, 96 nt, Artificial Sequence
  sgRNA for in vivo E. coli assays targeting 5′ CDS of DsRed
ugcaccuugaagcgcaugaaguuuuagagcuagaaauagcaaguuaaaauaaggcuaguccguuaucaacuugaaaaaguggcaccgagucggugc

SEQ ID NO: 24 - RNA, 96 nt, Artificial Sequence
  sgRNA for in vivo E. coli assays targeting 5′ CDS of DsRed, mismatch at position 1 (counting from PAM)
ugcaccuugaagcgcaugauguuuuagagcuagaaauagcaaguuaaaauaaggcuaguccguuaucaacuugaaaaaguggcaccgagucggugc

SEQ ID NO: 25 - RNA, 96 nt, Artificial Sequence
  sgRNA for in vivo E. coli assays targeting 5′ CDS of DsRed, mismatch at position 2 (counting from PAM)
ugcaccuugaagcgcauguaguuuuagagcuagaaauagcaaguuaaaauaaggcuaguccguuaucaacuugaaaaaguggcaccgagucggugc

SEQ ID NO: 26 - RNA, 96 nt, Artificial Sequence
  sgRNA for in vivo E. coli assays targeting 5′ CDS of DsRed, mismatch at position 3 (counting from PAM)
ugcaccuugaagcgcaucaaguuuuagagcuagaaauagcaaguuaaaauaaggcuaguccguuaucaacuugaaaaaguggcaccgagucggugc

SEQ ID NO: 27 - RNA, 96 nt, Artificial Sequence
  sgRNA for in vivo E. coli assays targeting 5′ CDS of DsRed, mismatch at position 4 (counting from PAM)
ugcaccuugaagcgcaagaaguuuuagagcuagaaauagcaaguuaaaauaaggcuaguccguuaucaacuugaaaaaguggcaccgagucggugc

SEQ ID NO: 28 - RNA, 96 nt, Artificial Sequence
  sgRNA for in vivo E. coli assays targeting 5′ CDS of DsRed, mismatch at position 5 (counting from PAM)
ugcaccuugaagcgcuugaaguuuuagagcuagaaauagcaaguuaaaauaaggcuaguccguuaucaacuugaaaaaguggcaccgagucggugc

SEQ ID NO: 29 - RNA, 96 nt, Artificial Sequence
  sgRNA for in vivo E. coli assays targeting 5′ CDS of DsRed, mismatch at position 6 (counting from PAM)
ugcaccuugaagcggaugaaguuuuagagcuagaaauagcaaguuaaaauaaggcuaguccguuaucaacuugaaaaaguggcaccgagucggugc

SEQ ID NO: 30 - RNA, 96 nt, Artificial Sequence
  sgRNA for in vivo E. coli assays targeting 5′ CDS of DsRed, mismatch at position 7 (counting from PAM)
ugcaccuugaagcccaugaaguuuuagagcuagaaauagcaaguuaaaauaaggcuaguccguuaucaacuugaaaaaguggcaccgagucggugc

SEQ ID NO: 31 - RNA, 96 nt, Artificial Sequence
  sgRNA for in vivo E. coli assays targeting 5′ CDS of DsRed, mismatch at position 8 (counting from PAM)
ugcaccuugaagggcaugaaguuuuagagcuagaaauagcaaguuaaaauaaggcuaguccguuaucaacuugaaaaaguggcaccgagucggugc

SEQ ID NO: 32 - RNA, 96 nt, Artificial Sequence
  sgRNA for in vivo E. coli assays targeting 5′ CDS of DsRed, mismatch at position 9 (counting from PAM)
ugcaccuugaaccgcaugaaguuuuagagcuagaaauagcaaguuaaaauaaggcuaguccguuaucaacuugaaaaaguggcaccgagucggugc

SEQ ID NO: 33 - RNA, 96 nt, Artificial Sequence
  sgRNA for in vivo E. coli assays targeting 5′ CDS of DsRed, mismatch at position 10 (counting from PAM)
ugcaccuugaugcgcaugaaguuuuagagcuagaaauagcaaguuaaaauaaggcuaguccguuaucaacuugaaaaaguggcaccgagucggugc

SEQ ID NO: 34 - RNA, 96 nt, Artificial Sequence
  sgRNA for in vivo E. coli assays targeting 5′ CDS of DsRed, mismatch at position 11 (counting from PAM)
ugcaccuuguagcgcaugaaguuuuagagcuagaaauagcaaguuaaaauaaggcuaguccguuaucaacuugaaaaaguggcaccgagucggugc

SEQ ID NO: 35 - RNA, 96 nt, Artificial Sequence
  sgRNA for in vivo E. coli assays targeting 5′ CDS of DsRed, mismatch at position 12 (counting from PAM)
ugcaccuucaagcgcaugaaguuuuagagcuagaaauagcaaguuaaaauaaggcuaguccguuaucaacuugaaaaaguggcaccgagucggugc

SEQ ID NO: 36 - RNA, 96 nt, Artificial Sequence
  sgRNA for in vivo E. coli assays targeting 5′ CDS of DsRed, mismatch at position 13 (counting from PAM)
ugcaccuagaagcgcaugaaguuuuagagcuagaaauagcaaguuaaaauaaggcuaguccguuaucaacuugaaaaaguggcaccgagucggugc

SEQ ID NO: 37 - RNA, 96 nt, Artificial Sequence
  sgRNA for in vivo E. coli assays targeting 5′ CDS of DsRed, mismatch at position 14 (counting from PAM)
ugcaccaugaagcgcaugaaguuuuagagcuagaaauagcaaguuaaaauaaggcuaguccguuaucaacuugaaaaaguggcaccgagucggugc

SEQ ID NO: 38 - RNA, 96 nt, Artificial Sequence
  sgRNA for in vivo E. coli assays targeting 5′ CDS of DsRed, mismatch at position 15 (counting from PAM)
ugcacguugaagcgcaugaaguuuuagagcuagaaauagcaaguuaaaauaaggcuaguccguuaucaacuugaaaaaguggcaccgagucggugc

SEQ ID NO: 39 - RNA, 96 nt, Artificial Sequence
  sgRNA for in vivo E. coli assays targeting 5′ CDS of DsRed, mismatch at position 16 (counting from PAM)
ugcagcuugaagcgcaugaaguuuuagagcuagaaauagcaaguuaaaauaaggcuaguccguuaucaacuugaaaaaguggcaccgagucggugc

SEQ ID NO: 40 - RNA, 96 nt, Artificial Sequence
  sgRNA for in vivo E. coli assays targeting 5′ CDS of DsRed, mismatch at position 17 (counting from PAM)
ugcuccuugaagcgcaugaaguuuuagagcuagaaauagcaaguuaaaauaaggcuaguccguuaucaacuugaaaaaguggcaccgagucggugc

SEQ ID NO: 41 - RNA, 96 nt, Artificial Sequence
  sgRNA for in vivo E. coli assays targeting 5′ CDS of DsRed, mismatch at position 18 (counting from PAM)
uggaccuugaagcgcaugaaguuuuagagcuagaaauagcaaguuaaaauaaggcuaguccguuaucaacuugaaaaaguggcaccgagucggugc

SEQ ID NO: 42 - RNA, 96 nt, Artificial Sequence
  sgRNA for in vivo E. coli assays targeting 5′ CDS of DsRed, mismatch at position 19 (counting from PAM)
uccaccuugaagcgcaugaaguuuuagagcuagaaauagcaaguuaaaauaaggcuaguccguuaucaacuugaaaaaguggcaccgagucggugc

SEQ ID NO: 43 - RNA, 96 nt, Artificial Sequence
  sgRNA for in vivo E. coli assays targeting 5′ CDS of DsRed, mismatch at position 20 (counting from PAM)
agcaccuugaagcgcaugaaguuuuagagcuagaaauagcaaguuaaaauaaggcuaguccguuaucaacuugaaaaaguggcaccgagucggugc

SEQ ID NO: 44 - DNA, 33 bp, Artificial Sequence
  speM protospacer used for in vitro experiments (PAM)
ttatatgaacataactcaatttgtaaaaaatgg

SEQ ID NO: 45 - DNA, 33 bp, Artificial Sequence
  speM protospacer used for in vitro experiments, mismatch to crRNA at position 1 (counted from PAM)
ttatatgaacataactcaatttgtaaaaattgg

SEQ ID NO: 46 - DNA, 33 bp, Artificial Sequence
  speM protospacer used for in vitro experiments, mismatch to crRNA at position 2 (counted from PAM)
ttatatgaacataactcaatttgtaaaatatgg

SEQ ID NO: 47 - DNA, 33 bp, Artificial Sequence
  speM protospacer used for in vitro experiments, mismatch to crRNA at position 3 (counted from PAM)
ttatatgaacataactcaatttgtaaataatgg

SEQ ID NO: 48 - DNA, 33 bp, Artificial Sequence
  speM protospacer used for in vitro experiments, mismatch to crRNA at position 4 (counted from PAM)
ttatatgaacataactcaatttgtaataaatgg

SEQ ID NO: 49 - DNA, 33 bp, Artificial Sequence
  speM protospacer used for in vitro experiments, mismatch to crRNA at position 5 (counted from PAM)
ttatatgaacataactcaatttgtataaaatgg

SEQ ID NO: 50 - DNA, 33 bp, Artificial Sequence
  speM protospacer used for in vitro experiments, mismatch to crRNA at position 6 (counted from PAM)
ttatatgaacataactcaatttgttaaaaatgg

SEQ ID NO: 51 - DNA, 33 bp, Artificial Sequence
  speM protospacer used for in vitro experiments, mismatch to crRNA at position 7 (counted from PAM)
ttatatgaacataactcaatttgaaaaaaatgg

SEQ ID NO: 52 - DNA, 33 bp, Artificial Sequence
  speM protospacer used for in vitro experiments, mismatch to crRNA at position 8 (counted from PAM)
ttatatgaacataactcaatttctaaaaaatgg

SEQ ID NO: 53 - DNA, 33 bp, Artificial Sequence
  speM protospacer used for in vitro experiments, mismatch to crRNA at position 9 (counted from PAM)
ttatatgaacataactcaattagtaaaaaatgg

SEQ ID NO: 54 - DNA, 33 bp, Artificial Sequence
  speM protospacer used for in vitro experiments, mismatch to crRNA at position 10 (counted from PAM)
ttatatgaacataactcaatatgtaaaaaatgg

SEQ ID NO: 55 - DNA, 33 bp, Artificial Sequence
  speM protospacer used for in vitro experiments, mismatch to crRNA at position 11 (counted from PAM)
ttatatgaacataactcaaattgtaaaaaatgg

SEQ ID NO: 56 - DNA, 33 bp, Artificial Sequence
  speM protospacer used for in vitro experiments, mismatch to crRNA at position 12 (counted from PAM)
ttatatgaacataactcattttgtaaaaaatgg

SEQ ID NO: 57 - DNA, 33 bp, Artificial Sequence
  speM protospacer used for in vitro experiments, mismatch to crRNA at position 13 (counted from PAM)
ttatatgaacataactctatttgtaaaaaatgg

SEQ ID NO: 58 - DNA, 33 bp, Artificial Sequence
  speM protospacer used for in vitro experiments, mismatch to crRNA at position 14 (counted from PAM)
ttatatgaacataactgaatttgtaaaaaatgg

SEQ ID NO: 59 - DNA, 33 bp, Artificial Sequence
  speM protospacer used for in vitro experiments, mismatch to crRNA at position 15 (counted from PAM)
ttatatgaacataacacaatttgtaaaaaatgg

SEQ ID NO: 60 - DNA, 33 bp, Artificial Sequence
  speM protospacer used for in vitro experiments, mismatch to crRNA at position 16 (counted from PAM)
ttatatgaacataagtcaatttgtaaaaaatgg

SEQ ID NO: 61 - DNA, 33 bp, Artificial Sequence
  speM protospacer used for in vitro experiments, mismatch to crRNA at position 17 (counted from PAM)
ttatatgaacatatctcaatttgtaaaaaatgg

SEQ ID NO: 62 - DNA, 33 bp, Artificial Sequence
  speM protospacer used for in vitro experiments, mismatch to crRNA at position 18 (counted from PAM)
ttatatgaacattactcaatttgtaaaaaatgg

SEQ ID NO: 63 - DNA, 33 bp, Artificial Sequence
  speM protospacer used for in vitro experiments, mismatch to crRNA at position 19 (counted from PAM)
ttatatgaacaaaactcaatttgtaaaaaatgg

SEQ ID NO: 64 - DNA, 33 bp, Artificial Sequence
  speM protospacer used for in vitro experiments, mismatch to crRNA at position 20 (counted from PAM)
ttatatgaacttaactcaatttgtaaaaaatgg

SEQ ID NO: 65 - DNA, 23 bp, Artificial Sequence
  DSRed target sequence used in in vivo E.coli experiements (PAM )
tgcaccttgaagcgcatgaaggg

SEQ ID NO: 66 - RNA, 20 nt, Artificial Sequence
  sgEpCAM-1
gatcctgactgcgatgagag

SEQ ID NO: 67 - RNA, 20 nt, Artificial Sequence
  sgEpCAM-2
gatcacaacgcgttatcaac

SEQ ID NO: 68 - RNA, 20 nt, Artificial Sequence
  sgEpCAM-3
taatgttatcactattgatc

SEQ ID NO: 69 - RNA, 20 nt, Artificial Sequence
  sgEpCAM-4
caagcagaagcccgaacgcg

SEQ ID NO: 70 - DNA, 33 bp, Artificial Sequence
  non-target strand
tgaacataactcaatttgtaaaaaatcccgcag

SEQ ID NO: 71 - DNA, 33 bp, Artificial Sequence
  target strand
ctgcgccattttttacaaattgagttatgttca

SEQ ID NO: 72 - RNA, 28 nt, Artificial Sequence
  crRNA strand
auaacucaauuuguaaaaaaguuuuaga

SEQ ID NO: 73 - DNA, 33 bp, Artificial Sequence
  no bubble
tgaacataactcaatttgtaaaaaatggtactc

SEQ ID NO: 74 - DNA, 33 bp, Artificial Sequence
  no bubble
gagtaccattttttacaaattgagttatgttca

SEQ ID NO: 75 - DNA, 33 bp, Artificial Sequence
  5 nt bubble
tgaacataactcaatttgtattttttggtactc

SEQ ID NO: 76 - DNA, 33 bp, Artificial Sequence; identical to SEQ ID NO: 74
  5 nt bubble

SEQ ID NO: 77 - DNA, 33 bp, Artificial Sequence
  20 nt bubble
tgaacagccagaccgggtgcccccctggtactc

SEQ ID NO: 78 - DNA, 33 bp, Artificial Sequence; identical to SEQ ID NO: 74
  20 nt bubble